(19) United States
(12) Patent Application Publication
(10) Pub. No.: US 2025/0011306 A1
(43) Pub. Date: Jan. 9, 2025

(54) ORGANIC COMPOUNDS AND ORGANIC LIGHT-EMITTING DEVICE COMPRISING THE SAME

(71) Applicant: SFC CO., LTD, Cheongju-si (KR)

(72) Inventors: Myeong-jun KIM, Cheongju-si (KR); Ho-jeong CHOI, Cheongju-si (KR); Jae-gyeong JEONG, Cheongju-si (KR); Bong-ki SHIN, Cheongju-si (KR)

(73) Assignee: SFC CO., LTD, Cheongju-si (KR)

(21) Appl. No.: 18/422,161

(22) Filed: Jan. 25, 2024

(30) Foreign Application Priority Data
Feb. 1, 2023 (KR) 10-2023-0013449
Jan. 16, 2024 (KR) 10-2024-0007036

(51) Int. Cl. C07D 405/04; C07B 59/00; C07D 405/14; C07D 409/10; C07D 409/14; C09K 11/06; H10K 50/12; H10K 85/60

(52) U.S. Cl. CPC C07D 405/04; C07B 59/004; C07D 405/14; C07D 409/10; C07D 409/14; C09K 11/06; H10K 50/12; H10K 85/622; H10K 85/623; H10K 85/654; H10K 85/6574; H10K 85/6576; C07B 2200/05

(57) ABSTRACT

Disclosed is an organic compound represented by Formula 1:

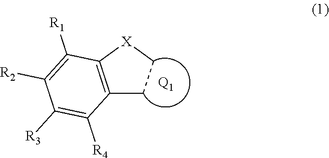

wherein Q₁ is

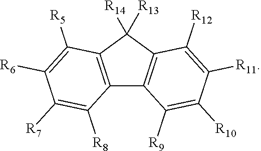

The organic compound has a fused structure as a skeleton and contains specific moieties introduced to the skeleton. The organic compound is employed in an organic layer (for example, a light emitting layer) of an organic light emitting device to achieve significantly improved low-voltage driving, long lifetime, and high luminous efficiency of the device. That is, the use of the organic compound makes the device highly efficient and long lasting.

### BACKGROUND OF THE INVENTION

### 1. Field of the Invention

The present invention relates to an organic compound employed in an organic layer of an organic light emitting device and an organic light emitting device including the organic compound.

### 2. Description of the Related Art

Organic light emitting devices are self-luminous devices in which electrons injected from an electron injecting electrode (cathode) recombine with holes injected from a hole injecting electrode (anode) in a light emitting layer to form excitons, which emit light while releasing energy. Such organic light emitting devices have the advantages of low driving voltage, high luminance, large viewing angle, and short response time and can be applied to full-color light emitting flat panel displays. Due to these advantages, organic light emitting devices have received attention as next-generation light sources.

The above characteristics of organic light emitting devices are achieved by structural optimization of organic layers of the devices and are supported by stable and efficient materials for the organic layers, such as hole injecting materials, hole transport materials, light emitting materials, electron transport materials, electron injecting materials, and electron blocking materials. However, more research still needs to be done to develop structurally optimized structures of organic layers for organic light emitting devices and stable and efficient materials for organic layers of organic light emitting devices.

Particularly, for maximum efficiency in a light emitting layer, an appropriate combination of energy band gaps of a host and a dopant is required such that holes and electrons migrate to the dopant through stable electrochemical paths to form excitons.

### SUMMARY OF THE INVENTION

Therefore, the present invention is intended to provide a host material with a specific structure for a light emitting layer and a light emitting device which includes the host material, achieving significantly improved low-voltage driving, long lifetime, and high luminous efficiency.

One aspect of the present invention provides an organic compound represented by Formula 1:

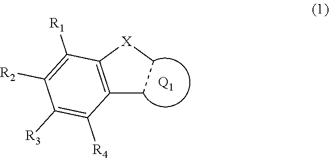

wherein Q₁ is

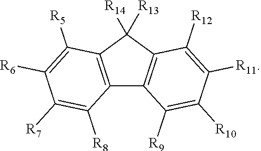

The organic compound has a fused structure as a skeleton and contains specific moieties introduced to the skeleton.

A further aspect of the present invention provides an organic light emitting device including the organic compound as a host for a light emitting layer.

The specific structure of Formula 1, definitions of the substituents in Formula 1, and specific compounds that can be represented by Formula 1 are described below.

The organic light emitting device of the present invention includes a light emitting layer in which the organic compound having a fused structure as a skeleton and containing specific moieties introduced to the skeleton is employed as a host. The use of the host ensures improved low-voltage driving, high efficiency, and long lifetime of the organic light emitting device. Due to these advantages, the organic light emitting device of the present invention can find useful applications in not only lighting systems but also a variety of displays, including flat panel displays, flexible displays, and wearable displays.

### DETAILED DESCRIPTION OF THE INVENTION

The present invention will now be described in more detail.

One aspect of the present invention is directed to a compound represented by Formula 1:

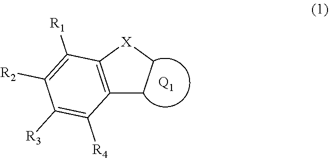

wherein X is O or S, Q₁ is

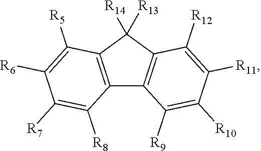

one of the pairs of the adjacent carbon atoms to which R₉ and R₁₀, R₁₀ and R₁₁, and R₁₁ and R₁₂ are attached is condensed with the pair of the adjacent carbon atoms connected by the dashed line form a ring, R₁₃ and R₁₄ are the same as or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and cyclic groups in which a substituted or unsubstituted C₃-C₃₀ aliphatic ring and a C₅-C₃₀ aromatic ring are fused together, with the proviso that R₁₃ and R₁₄ are optionally linked to each other to form an alicyclic or aromatic mono- or polycyclic ring, and the remainders of R₉ to R₁₂ that do not participate in the ring formation and R₁ to R₈ are the same as or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, cyclic groups in which a substituted or unsubstituted C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, with the proviso that each of R₁ to R₁₂ is optionally linked to an adjacent substituent to form an alicyclic or aromatic mono- or polycyclic ring and that at least one of R₁ to R₁₂ is linked to Structural Formula 1:

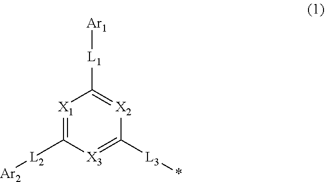

wherein * represents a site at which one of R₁ to R₁₂ and L₃ form a single bond, X₁ to X₃ are the same as or different from each other and are each independently CR or N, with the proviso that at least one of X₁ to X₃ is N, each R is independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and cyclic groups in which a substituted or unsubstituted C₃-C₃₀ aliphatic ring and a C₅-C₃₀ aromatic ring are fused together, L₁ to L₃ are the same as or different from each other and are each independently a single bond or selected from substituted or unsubstituted C₆-C₃₀ arylene, substituted or unsubstituted C₂-C₃₀ heteroarylene, and divalent cyclic groups in which a substituted or unsubstituted C₃-C₂₄ aliphatic ring and a C₅-C₂₄ aromatic ring are fused together, Ar₁ is selected from substituted or unsubstituted C₆-C₃₀ aryl, substituted or unsubstituted C₂-C₃₀ heteroaryl, and cyclic groups in which a substituted or unsubstituted C₃-C₂₄ aliphatic ring and a C₅-C₂₄ aromatic ring are fused together, and Ar₂ is represented by Structural Formula 2:

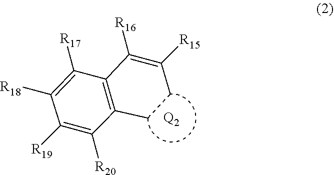

wherein Q₂ is represented by one of Structural Formulas A, B, and C:

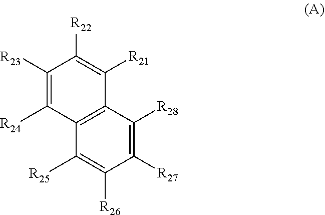

wherein one of the pairs of the adjacent carbon atoms to which R₂₁ and R₂₂, R₂₂ and R₂₃, and R₂₃ and R₂₄ are attached is condensed with the pair of the adjacent carbon atoms connected by the dashed line in Structural Formula 2 form a ring;

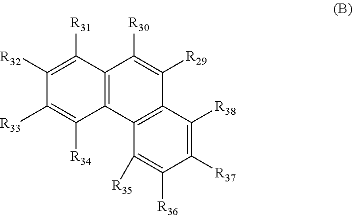

wherein one of the pairs of the adjacent carbon atoms to which R₂₉ and R₃₀, R₃₁ and R₃₂, R₃₂ and R₃₃, and R₃₃ and R₃₄ are attached is condensed with the pair of the adjacent carbon atoms connected by the dashed line in Structural Formula 2 form a ring; and

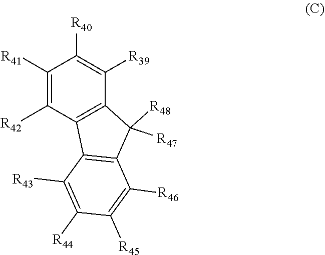

wherein one of the pairs of the adjacent carbon atoms to which R₃₉ and R₄₀, R₄₀ and R₄₁, and R₄₁ and R₄₂ are attached is condensed with the pair of the adjacent carbon atoms connected by the dashed line in Structural Formula 2 form a ring, R₄₇ and R₄₈ are the same as or different from each other and are each independently selected from hydrogen, deuterium, substituted or unsubstituted C₁-C₂₄ alkyl, substituted or unsubstituted C₂-C₂₄ alkynyl, substituted or unsubstituted C₂-C₂₄ alkenyl, substituted or unsubstituted C₆-C₃₀ aryl, substituted or unsubstituted C₃-C₃₀ cycloalkyl, substituted or unsubstituted C₂-C₃₀ heterocycloalkyl, substituted or unsubstituted C₂-C₃₀ heteroaryl, and cyclic groups in which a substituted or unsubstituted C₃-C₂₄ aliphatic ring and a C₅-C₂₄ aromatic ring are fused together, with the proviso that R₄₇ and R₄₈ are optionally linked to each other to form an alicyclic or aromatic mono- or polycyclic ring, the remainders of R₁₅ to R₄₆ that do not participate in the ring formation are the same as or different from each other and are each independently selected from hydrogen, deuterium, substituted or unsubstituted C₁-C₂₄ alkyl, substituted or unsubstituted C₂-C₂₄ alkynyl, substituted or unsubstituted C₂-C₂₄ alkenyl, substituted or unsubstituted C₆-C₃₀ aryl, substituted or unsubstituted C₃-C₃₀ cycloalkyl, substituted or unsubstituted C₂-C₃₀ heterocycloalkyl, substituted or unsubstituted C₂-C₃₀ heteroaryl, cyclic groups in which a substituted or unsubstituted C₃-C₂₄ aliphatic ring and a C₅-C₂₄ aromatic ring are fused together, substituted or unsubstituted C₁-C₂₄ alkoxy, substituted or unsubstituted C₆-C₂₄ aryloxy, substituted or unsubstituted C₁-C₂₄ alkylthioxy, substituted or unsubstituted C₅-C₂₄ arylthioxy, substituted or unsubstituted amine, substituted or unsubstituted silyl, substituted or unsubstituted germanium, nitro, cyano, and halogen, with the proviso that each of R₁₅ to R₄₈ is optionally linked to an adjacent substituent to form an alicyclic or aromatic mono- or polycyclic ring.

According to one embodiment of the present invention, X₁ to X₃ in Structural Formula 1 may be all N.

According to one embodiment of the present invention, Formula 1 may be selected from Formulas 1-1 to 1-6:

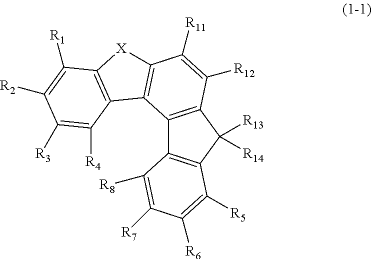

wherein R₁ to R₈, R₁₁ to R₁₄, and X are as defined in Formula 1;

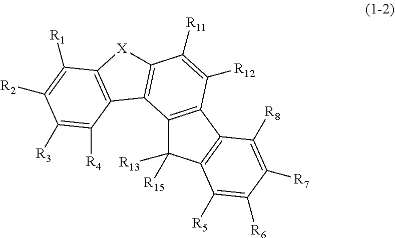

wherein R₁ to R₁₀, R₁₃, R₁₄, and X are as defined in Formula 1;

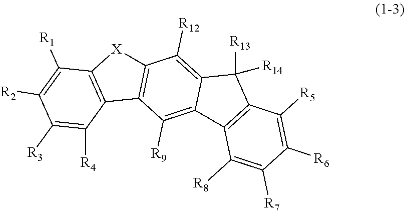

wherein R₁ to R₉, R₁₂ to R₁₄, and X are as defined in Formula 1;

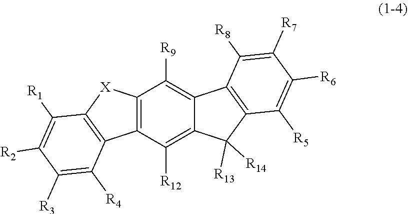

wherein R₁ to R₉, R₁₂ to R₁₄, and X are as defined in Formula 1;

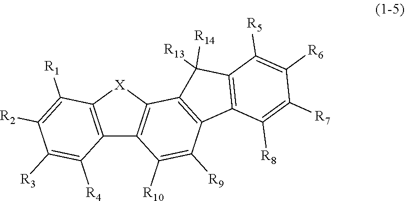

wherein R₁ to R₁₀, R₁₃, R₁₄, and X are as defined in Formula 1; and

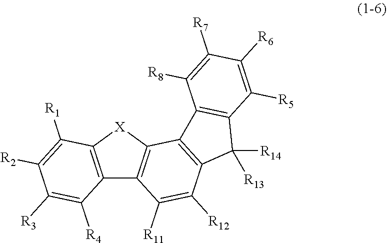

wherein R₁ to R₈, R₁₁ to R₁₄, and X are as defined in Formula 1.

According to one embodiment of the present invention, Structural Formula 2 may be selected from Structural Formulas A-1 to A-3, B-1 to B-6, and C-1 to C-6:

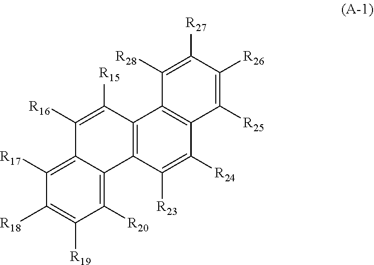

wherein R₁₅ to R₂₀ and R₂₃ to R₂₈ are as defined in Formula 1;

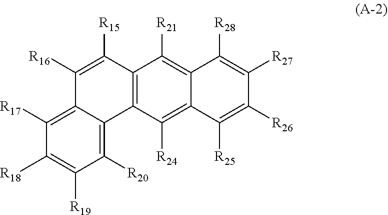

wherein R₁₅ to R₂₁ and R₂₄ to R₂₈ are as defined in Formula 1;

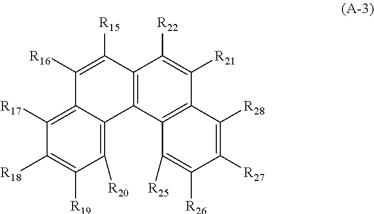

wherein R₁₅ to R₂₂ and R₂₅ to R₂₈ are as defined in Formula 1;

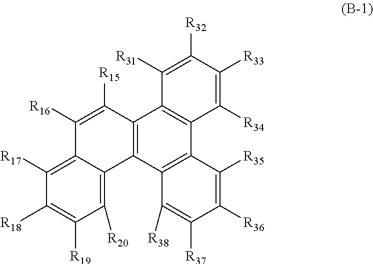

wherein R₁₅ to R₂₀ and R₃₁ to R₃₈ are as defined in Formula 1;

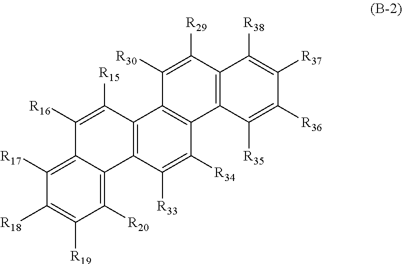

wherein R₁₅ to R₂₀, R₂₉, R₃₀, and R₃₃ to R₃₈ are as defined in Formula 1;

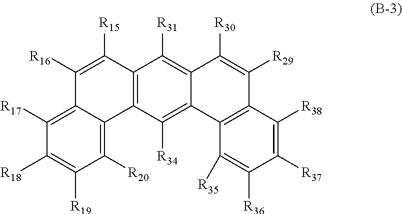

wherein R₁₅ to R₂₀, R₂₉ to R₃₁, and R₃₄ to R₃₈ are as defined in Formula 1;

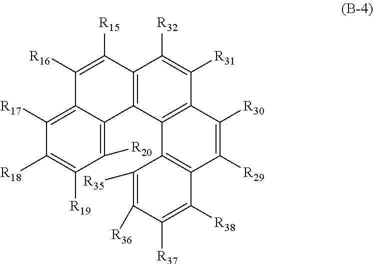

wherein R₁₅ to R₂₀, R₂₉ to R₃₂, and R₃₅ to R₃₈ are as defined in Formula 1;

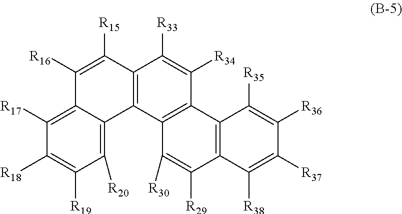

wherein R₁₅ to R₂₀, R₂₉, R₃₀, and R₃₃ to R₃₈ are as defined in Formula 1;

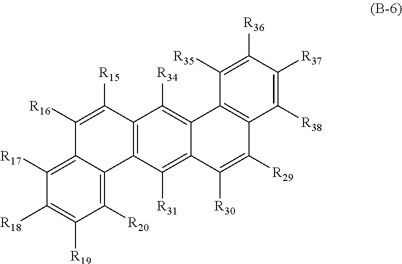

wherein R₁₅ to R₂₀, R₂₉ to R₃₁, and R₃₄ to R₃₈ are as defined in Formula 1;

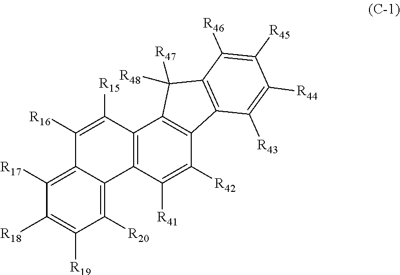

wherein R₁₅ to R₂₀ and R₄₁ to R₄₈ are as defined in Formula 1;

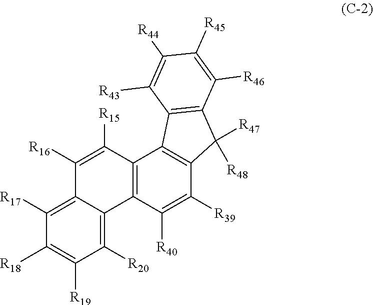

wherein R₁₅ to R₂₀, R₃₉, R₄₀, and R₄₃ to R₄₈ are as defined in Formula 1;

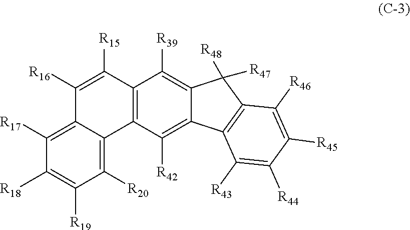

wherein R₁₅ to R₂₀, R₃₉, and R₄₂ to R₄₈ are as defined in Formula 1;

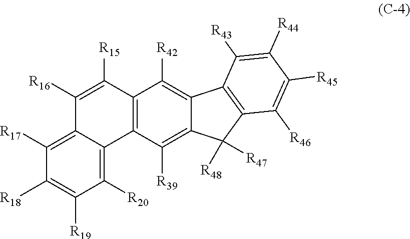

wherein R₁₅ to R₂₀, R₃₉, and R₄₂ to R₄₈ are as defined in Formula 1;

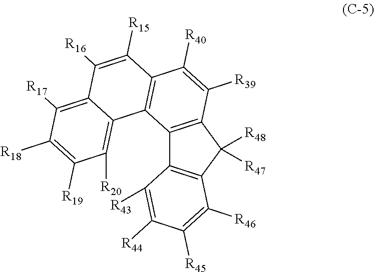

wherein R₁₅ to R₂₀, R₃₉, R₄₀, and R₄₃ to R₄₈ are as defined in Formula 1; and

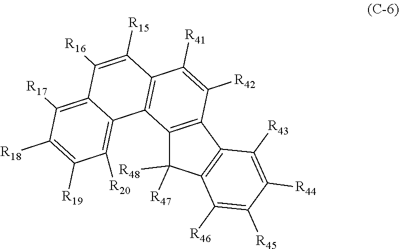

wherein R₁₅ to R₂₀ and R₄₁ to R₄₈ are as defined in Formula 1.

As used herein, the term “substituted” in the definitions of R₁ to R₁₄ in Formula 1 and Structural Formulas 1, 2, A, B, and C indicates substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, cyclic groups in which a C₃-C₂₄ aliphatic ring and a C₃-C₂₄ aromatic ring are fused together, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof. The term “unsubstituted” in the same definition indicates having no substituent. One or more hydrogen atoms in each of the substituents are optionally replaced by deuterium atoms and two or more adjacent ones of the substituents are optionally linked to each other to form an alicyclic or aromatic mono- or polycyclic ring.

In the “substituted or unsubstituted C₁-C₃₀ alkyl”, “substituted or unsubstituted C₆-C₅₀ aryl”, etc., the number of carbon atoms in the alkyl or aryl group indicates the number of carbon atoms constituting the unsubstituted alkyl or aryl moiety without considering the number of carbon atoms in the substituent(s). For example, a phenyl group substituted with a butyl group at the para-position corresponds to a C₆ aryl group substituted with a C₄ butyl group.

As used herein, the expression “optionally linked to each other or an adjacent group to form a ring” means that the corresponding adjacent substituents are bonded to each other or each of the corresponding substituents is bonded to an adjacent group to form a substituted or unsubstituted alicyclic or aromatic ring. The term “adjacent group” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other. Optionally, the paired substituents each lose one hydrogen radical and are linked to each other to form a ring. The carbon atoms in the resulting alicyclic, aromatic mono- or polycyclic ring may be replaced by one or more heteroatoms such as N, NR (wherein R is as defined in Structural Formula 1), O, S, Si, and Ge.

In the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

The alkenyl group is intended to include straight and branched ones and may be optionally substituted with one or more other substituents. The alkenyl group may be specifically a vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, stilbenyl or styrenyl group but is not limited thereto.

The alkynyl group is intended to include straight and branched ones and may be optionally substituted with one or more other substituents. The alkynyl group may be, for example, ethynyl or 2-propynyl but is not limited thereto.

The cycloalkenyl group is a non-aromatic cyclic unsaturated hydrocarbon group having one or more carbon-carbon double bonds. The cycloalkenyl group may be, for example, cyclopropenyl, cyclobutenyl, cyclopentenyl, cyclohexenyl, cycloheptenyl, 1,3-cyclohexadienyl, 1,4-cyclohexadienyl, 2,4-cycloheptadienyl or 1,5-cyclooctadienyl but is not limited thereto.

The aromatic hydrocarbon rings or aryl groups may be monocyclic or polycyclic ones. As used herein, the term “polycyclic” means that the aromatic hydrocarbon ring may be directly attached or fused to one or more other cyclic groups. The other cyclic groups may be aromatic hydrocarbon rings and other examples thereof include aliphatic heterocyclic rings, aliphatic hydrocarbon rings, and aromatic heterocyclic rings. Examples of the monocyclic aryl groups include, but are not limited to, phenyl, biphenyl, and terphenyl. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups but the scope of the present invention is not limited thereto.

The aromatic heterocyclic rings or heteroaryl groups refer to aromatic groups containing one or more heteroatoms. Examples of the aromatic heterocyclic rings or heteroaryl groups include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, and phenothiazinyl groups.

The aliphatic hydrocarbon rings or cycloalkyl groups refer to non-aromatic rings consisting only of carbon and hydrogen atoms. The aliphatic hydrocarbon ring is intended to include monocyclic and polycyclic ones and may be optionally substituted with one or more other substituents. As used herein, the term “polycyclic” means that the aliphatic hydrocarbon ring may be directly attached or fused to one or more other cyclic groups. The other cyclic groups may be aliphatic hydrocarbon rings and other examples thereof include aliphatic heterocyclic rings, aromatic hydrocarbon rings, and aromatic heterocyclic rings. Specific examples of the aliphatic hydrocarbon rings include, but are not limited to, cycloalkyl groups such as cyclopropyl, cyclobutyl, cyclopentyl, adamantyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, and cyclooctyl, cycloalkanes such as cyclohexane and cyclopentane, and cycloalkenes such as cyclohexene and cyclobutene.

The aliphatic heterocyclic rings or heterocycloalkyl groups refer to aliphatic rings containing one or more heteroatoms such as O, S, Se, N, and Si. The aliphatic heterocyclic ring is intended to include monocyclic or polycyclic ones and may be optionally substituted with one or more other substituents. As used herein, the term “polycyclic” means that the aliphatic heterocyclic ring such as heterocycloalkyl or heterocycloalkane may be directly attached or fused to one or more other cyclic groups. The other cyclic groups may be aliphatic heterocyclic rings and other examples thereof include aliphatic hydrocarbon rings, aromatic hydrocarbon rings, and aromatic heterocyclic rings.

The cyclic groups in which an aliphatic ring and an aromatic ring are fused together refers to mixed aliphatic-aromatic cyclic groups in which at least one aliphatic ring and at least one aromatic ring are linked and fused together and which are overall non-aromatic. More specifically, the cyclic groups in which an aliphatic ring and an aromatic ring are fused together may be an aromatic hydrocarbon cyclic group fused with an aliphatic hydrocarbon ring, an aromatic hydrocarbon cyclic group fused with an aliphatic heterocyclic ring, an aromatic heterocyclic group fused with an aliphatic hydrocarbon ring, an aromatic heterocyclic group fused with an aliphatic heterocyclic ring, an aliphatic hydrocarbon cyclic group fused with an aromatic hydrocarbon ring, an aliphatic hydrocarbon cyclic group fused with an aromatic hydrocarbon ring, an aliphatic heterocyclic group fused with an aromatic hydrocarbon ring, and an aliphatic heterocyclic group fused with an aromatic heterocyclic ring. Specific examples of the cyclic groups in which an aliphatic ring and an aromatic ring are fused together include tetrahydronaphthyl, tetrahydrobenzocycloheptene, tetrahydrophenanthrene, tetrahydroanthracenyl, octahydrotriphenylene, tetrahydrobenzothiophene, tetrahydrobenzofuranyl, tetrahydrocarbazole, and tetrahydroquinoline. The cyclic groups in which an aliphatic ring and an aromatic ring are fused together may be interrupted by contain at least one heteroatom other than carbon. The heteroatom may be, for example, N, NR (where R is as defined in Structural Formula 1), O, S, Si or Ge.

The alkoxy group may be specifically a methoxy, ethoxy, propoxy, isobutyloxy, sec-butyloxy, pentyloxy, iso-amyloxy or hexyloxy group but is not limited thereto.

The silyl group is intended to include —SiH₃, alkylsilyl, arylsilyl, alkylarylsilyl, arylheteroarylsilyl, and heteroarylsilyl. The arylsilyl refers to a silyl group obtained by substituting one, two or three of the hydrogen atoms in —SiH₃ with aryl groups. The alkylsilyl refers to a silyl group obtained by substituting one, two or three of the hydrogen atoms in —SiH₃ with alkyl groups. The alkylarylsilyl refers to a silyl group obtained by substituting one of the hydrogen atoms in —SiH₃ with an alkyl group and the other two hydrogen atoms with aryl groups or substituting two of the hydrogen atoms in —SiH₃ with alkyl groups and the remaining hydrogen atom with an aryl group. The arylheteroarylsilyl refers to a silyl group obtained by substituting one of the hydrogen atoms in —SiH₃ with an aryl group and the other two hydrogen atoms with heteroaryl groups or substituting two of the hydrogen atoms in —SiH₃ with aryl groups and the remaining hydrogen atom with a heteroaryl group. The heteroarylsilyl refers to a silyl group obtained by substituting one, two or three of the hydrogen atoms in —SiH₃ with heteroaryl groups. The arylsilyl group may be, for example, substituted or unsubstituted monoarylsilyl, substituted or unsubstituted diarylsilyl, or substituted or unsubstituted triarylsilyl. The same applies to the alkylsilyl and heteroarylsilyl groups.

Each of the aryl groups in the arylsilyl, heteroarylsilyl, and arylheteroarylsilyl groups may be a monocyclic or polycyclic one. Each of the heteroaryl groups in the arylsilyl, heteroarylsilyl, and arylheteroarylsilyl groups may be a monocyclic or polycyclic one.

Specific examples of the silyl groups include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more of the hydrogen atoms in each of the silyl groups may be substituted with the substituents mentioned in the aryl groups.

The amine group is intended to include —NH₂, alkylamine, arylamine, arylheteroarylamine, and heteroarylamine. The arylamine refers to an amine group obtained by substituting one or two of the hydrogen atoms in —NH₂ with aryl groups. The alkylamine refers to an amine group obtained by substituting one or two of the hydrogen atoms in —NH₂ with alkyl groups. The alkylarylamine refers to an amine group obtained by substituting one of the hydrogen atoms in —NH₂ with an alkyl group and the other hydrogen atom with an aryl group. The arylheteroarylamine refers to an amine group obtained by substituting one of the hydrogen atoms in —NH₂ with an aryl group and the other hydrogen atom with a heteroaryl group. The heteroarylamine refers to an amine group obtained by substituting one or two of the hydrogen atoms in —NH₂ with heteroaryl groups. The arylamine may be, for example, substituted or unsubstituted monoarylamine, substituted or unsubstituted diarylamine, or substituted or unsubstituted triarylamine. The same applies to the alkylamine and heteroarylamine groups.

Each of the aryl groups in the arylamine, heteroarylamine, and arylheteroarylamine groups may be a monocyclic or polycyclic one. Each of the heteroaryl groups in the arylamine, heteroarylamine, and arylheteroarylamine groups may be a monocyclic or polycyclic one.

The germanium group is intended to include —GeH₃, alkylgermanium, arylgermanium, heteroarylgermanium, alkylarylgermanium, alkylheteroarylgermanium, and arylheteroarylgermanium. The definitions of the substituents in the germanium groups follow those described for the silyl groups, except that the silicon (Si) atom in each silyl group is changed to a germanium (Ge) atom.

Specific examples of the germanium groups include trimethylgermane, triethylgermane, triphenylgermane, trimethoxygermane, dimethoxyphenylgermane, diphenylmethylgermane, diphenylvinylgermane, methylcyclobutylgermane, and dimethylfurylgermane. One or more of the hydrogen atoms in each of the germanium groups may be substituted with the substituents mentioned in the aryl groups.

The cycloalkyl, aryl, and heteroaryl groups in the cycloalkyloxy, aryloxy, heteroaryloxy, cycloalkylthioxy, arylthioxy, and heteroarylthioxy groups are the same as those exemplified above. Specific examples of the aryloxy groups include, but are not limited to, phenoxy, p-tolyloxy, m-tolyloxy, 3,5-dimethylphenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, and 9-phenanthryloxy groups. Specific examples of the arylthioxy groups include, but are not limited to, phenylthioxy, 2-methylphenylthioxy, and 4-tert-butylphenylthioxy groups.

The halogen group may be, for example, fluorine, chlorine, bromine or iodine.

According to one embodiment of the present invention, the compound represented by Formula 1 may be selected from the following compounds 1 to 180:

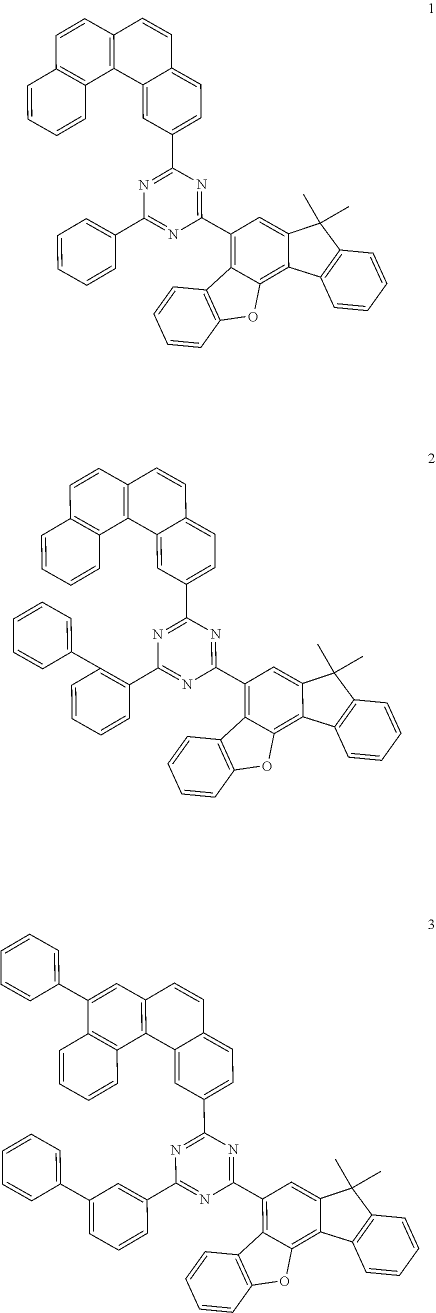 [SMILES: CC1(C)c2ccccc2-c2c1cc(-c1nc(-c3ccccc3)nc(-c3ccc4ccc5ccc6ccccc6c5c4c3)n1)c1c2oc2ccccc21 ; CC1(C)c2ccccc2-c2c1cc(-c1nc(-c3ccc4ccc5ccc6ccccc6c5c4c3)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5cc(-c6ccccc6)c6ccccc6c5c4c3)n1)c1c2oc2ccccc21]

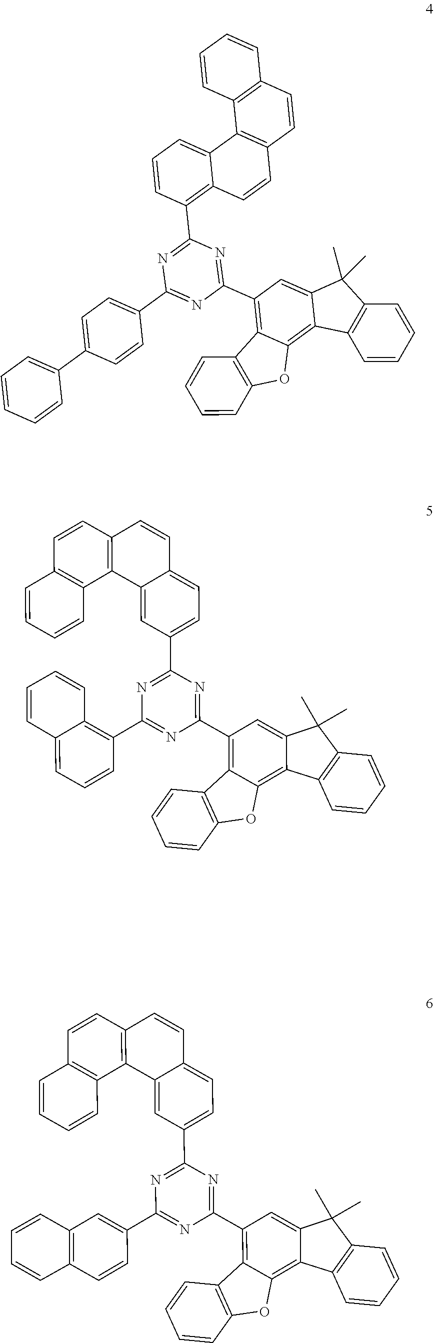 [SMILES: CC1(C)c2ccccc2-c2c1cc(-c1nc(-c3ccc(-c4ccccc4)cc3)nc(-c3cccc4c3ccc3ccc5ccccc5c34)n1)c1c2oc2ccccc21 ; CC1(C)c2ccccc2-c2c1cc(-c1nc(-c3ccc4ccc5ccc6ccccc6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6ccccc6c5c4c3)n1)c1c2oc2ccccc21]

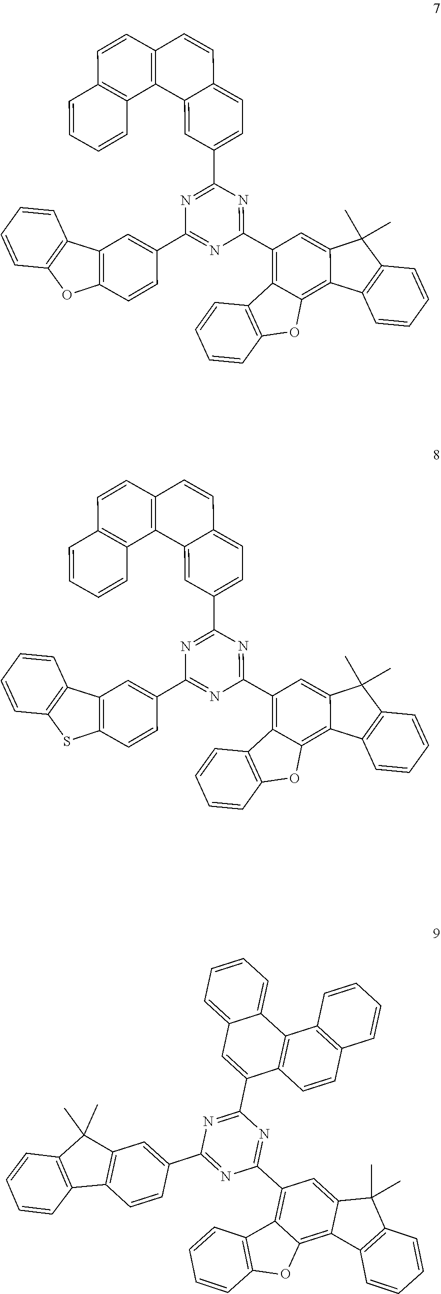 [SMILES: CC1(C)c2ccccc2-c2c1cc(-c1nc(-c3ccc4oc5ccccc5c4c3)nc(-c3ccc4ccc5ccc6ccccc6c5c4c3)n1)c1c2oc2ccccc21 ; CC1(C)c2ccccc2-c2c1cc(-c1nc(-c3ccc4sc5ccccc5c4c3)nc(-c3ccc4ccc5ccc6ccccc6c5c4c3)n1)c1c2oc2ccccc21 ; CC1(C)c2ccccc2-c2ccc(-c3nc(-c4cc5ccccc5c5c4ccc4ccccc45)nc(-c4cc5c(c6oc7ccccc7c46)-c4ccccc4C5(C)C)n3)cc21]

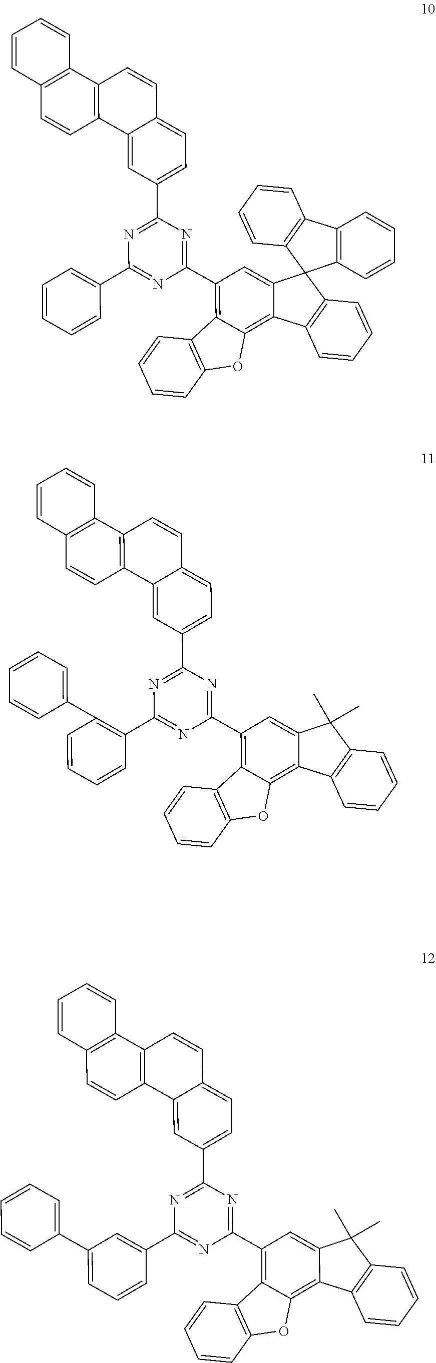 [SMILES: c1ccc(-c2nc(-c3ccc4ccc5c6ccccc6ccc5c4c3)nc(-c3cc4c(c5oc6ccccc6c35)-c3ccccc3C43c4ccccc4-c4ccccc43)n2)cc1 ; CC1(C)c2ccccc2-c2c1cc(-c1nc(-c3ccc4ccc5c6ccccc6ccc5c4c3)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5c6ccccc6ccc5c4c3)n1)c1c2oc2ccccc21]

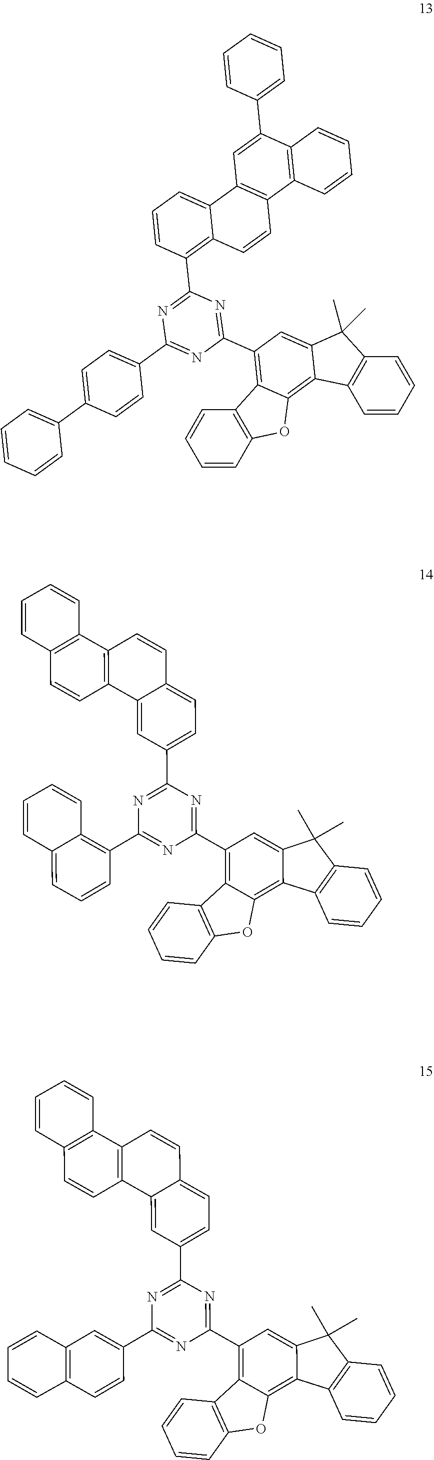 [SMILES: CC1(C)c2ccccc2-c2c1cc(-c1nc(-c3ccc(-c4ccccc4)cc3)nc(-c3cccc4c3ccc3c5ccccc5c(-c5ccccc5)cc43)n1)c1c2oc2ccccc21 ; CC1(C)c2ccccc2-c2c1cc(-c1nc(-c3ccc4ccc5c6ccccc6ccc5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5c6ccccc6ccc5c4c3)n1)c1c2oc2ccccc21]

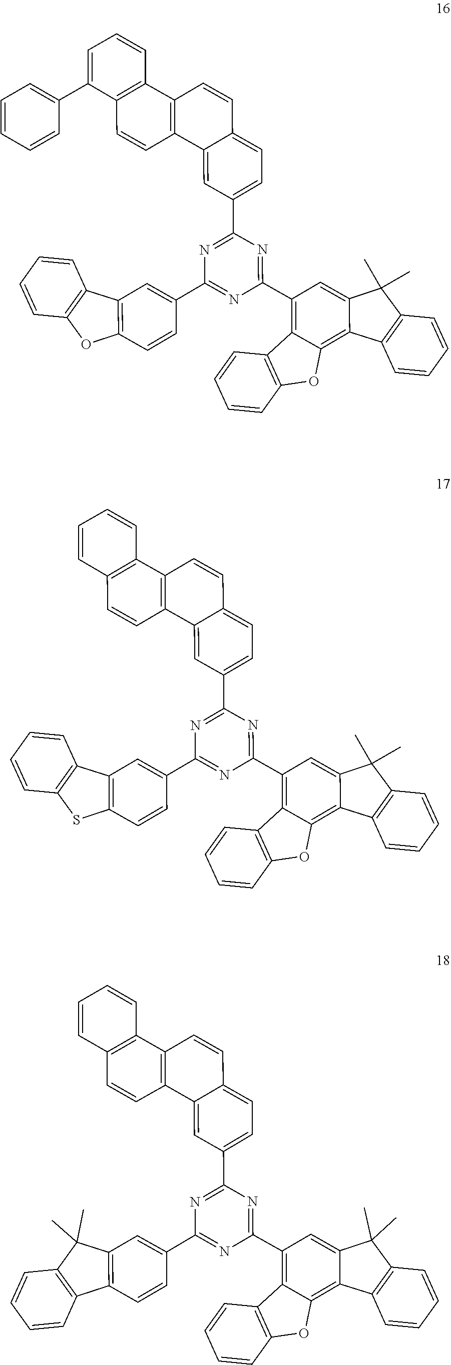 [SMILES: CC1(C)c2ccccc2-c2c1cc(-c1nc(-c3ccc4ccc5c6cccc(-c7ccccc7)c6ccc5c4c3)nc(-c3ccc4oc5ccccc5c4c3)n1)c1c2oc2ccccc21 ; CC1(C)c2ccccc2-c2c1cc(-c1nc(-c3ccc4ccc5c6ccccc6ccc5c4c3)nc(-c3ccc4sc5ccccc5c4c3)n1)c1c2oc2ccccc21 ; CC1(C)c2ccccc2-c2ccc(-c3nc(-c4ccc5ccc6c7ccccc7ccc6c5c4)nc(-c4cc5c(c6oc7ccccc7c46)-c4ccccc4C5(C)C)n3)cc21]

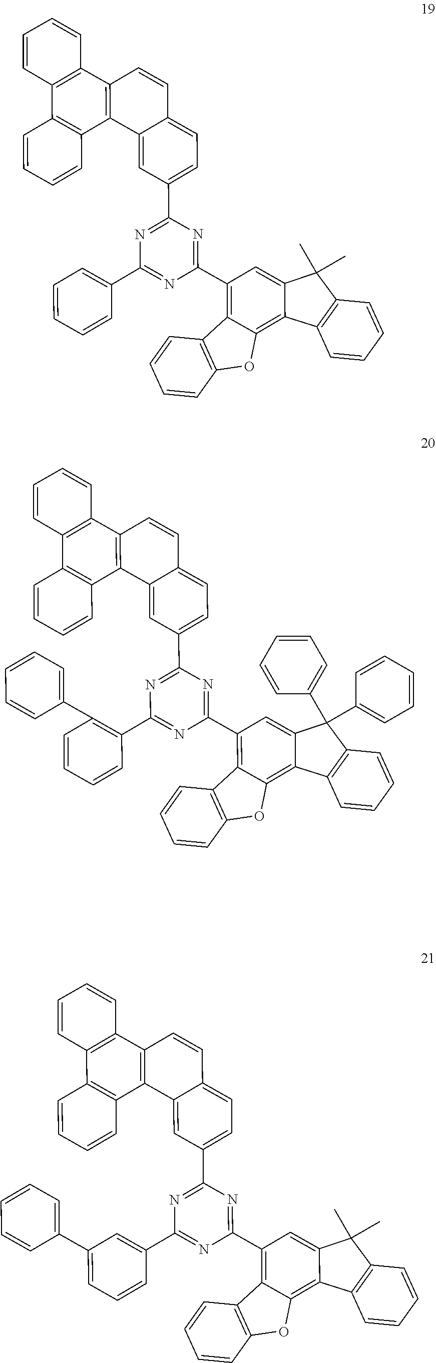 [SMILES: CC1(C)c2ccccc2-c2c1cc(-c1nc(-c3ccccc3)nc(-c3ccc4ccc5c6ccccc6c6ccccc6c5c4c3)n1)c1c2oc2ccccc21 ; c1ccc(-c2ccccc2-c2nc(-c3ccc4ccc5c6ccccc6c6ccccc6c5c4c3)nc(-c3cc4c(c5oc6ccccc6c35)-c3ccccc3C4(c3ccccc3)c3ccccc3)n2)cc1 ; CC1(C)c2ccccc2-c2c1cc(-c1nc(-c3cccc(-c4ccccc4)c3)nc(-c3ccc4ccc5c6ccccc6c6ccccc6c5c4c3)n1)c1c2oc2ccccc21]

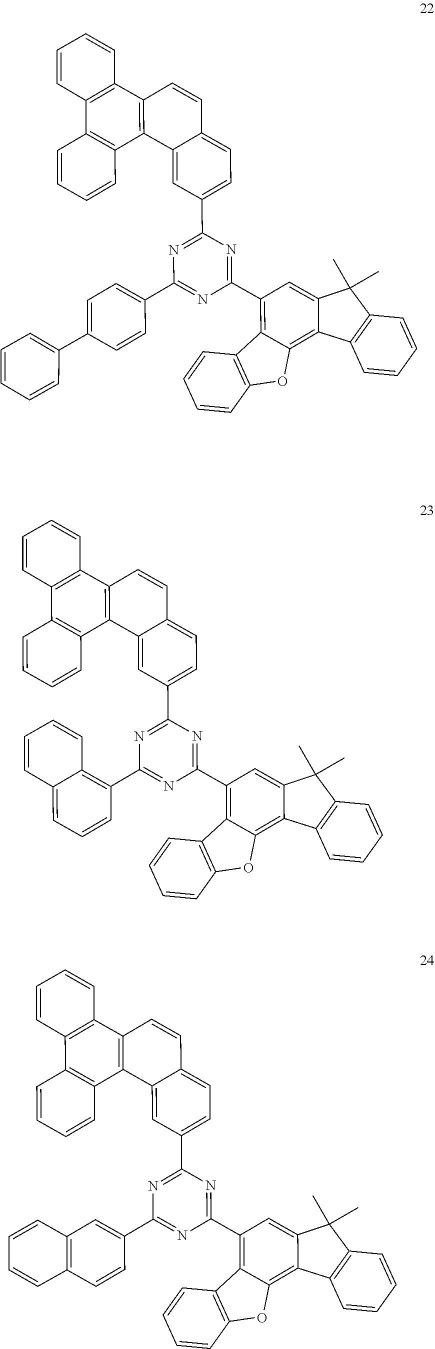 [SMILES: CC1(C)c2ccccc2-c2c1cc(-c1nc(-c3ccc(-c4ccccc4)cc3)nc(-c3ccc4ccc5c6ccccc6c6ccccc6c5c4c3)n1)c1c2oc2ccccc21 ; CC1(C)c2ccccc2-c2c1cc(-c1nc(-c3ccc4ccc5c6ccccc6c6ccccc6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5c6ccccc6c6ccccc6c5c4c3)n1)c1c2oc2ccccc21]

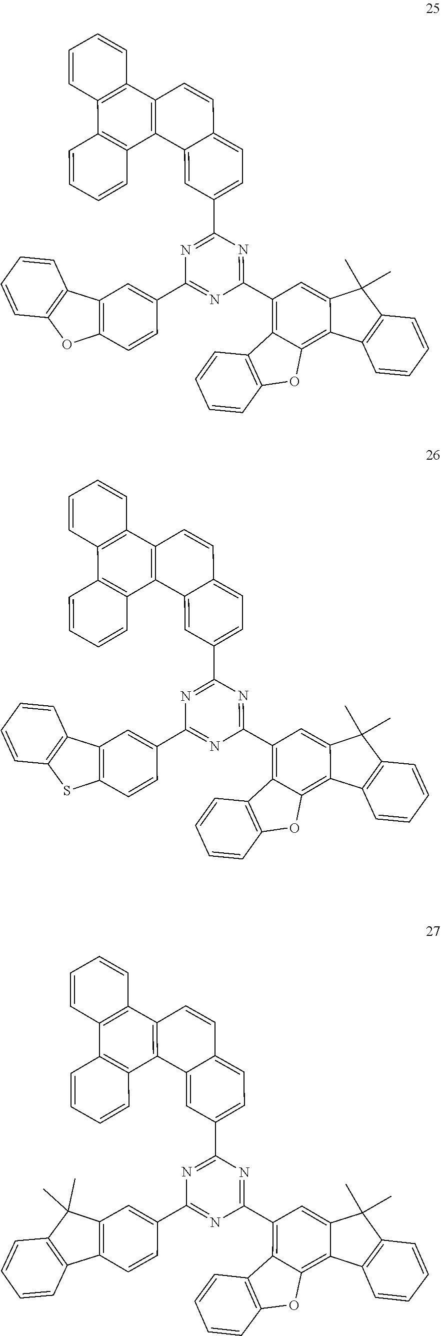 [SMILES: CC1(C)c2ccccc2-c2c1cc(-c1nc(-c3ccc4oc5ccccc5c4c3)nc(-c3ccc4ccc5c6ccccc6c6ccccc6c5c4c3)n1)c1c2oc2ccccc21 ; CC1(C)c2ccccc2-c2c1cc(-c1nc(-c3ccc4sc5ccccc5c4c3)nc(-c3ccc4ccc5c6ccccc6c6ccccc6c5c4c3)n1)c1c2oc2ccccc21 ; CC1(C)c2ccccc2-c2ccc(-c3nc(-c4ccc5ccc6c7ccccc7c7ccccc7c6c5c4)nc(-c4cc5c(c6oc7ccccc7c46)-c4ccccc4C5(C)C)n3)cc21]

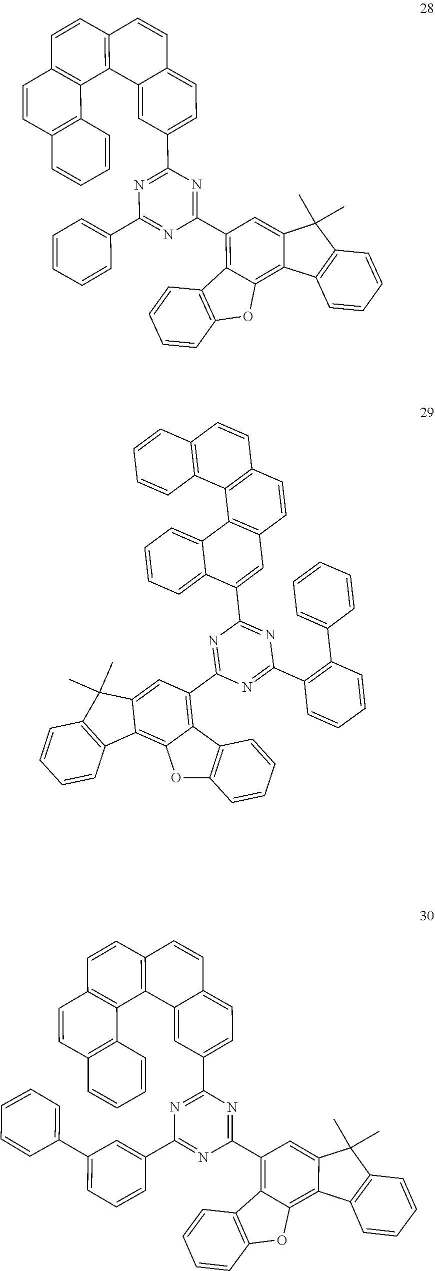 [SMILES: CC1(C)c2ccccc2-c2c1cc(-c1nc(-c3ccccc3)nc(-c3ccc4ccc5ccc6ccc7ccccc7c6c5c4c3)n1)c1c2oc2ccccc21 ; CC1(C)c2ccccc2-c2c1cc(-c1nc(-c3ccccc3-c3ccccc3)nc(-c3cc4ccc5ccc6ccccc6c5c4c4ccccc34)n1)c1c2oc2ccccc21 ; CC1(C)c2ccccc2-c2c1cc(-c1nc(-c3cccc(-c4ccccc4)c3)nc(-c3ccc4ccc5ccc6ccc7ccccc7c6c5c4c3)n1)c1c2oc2ccccc21]

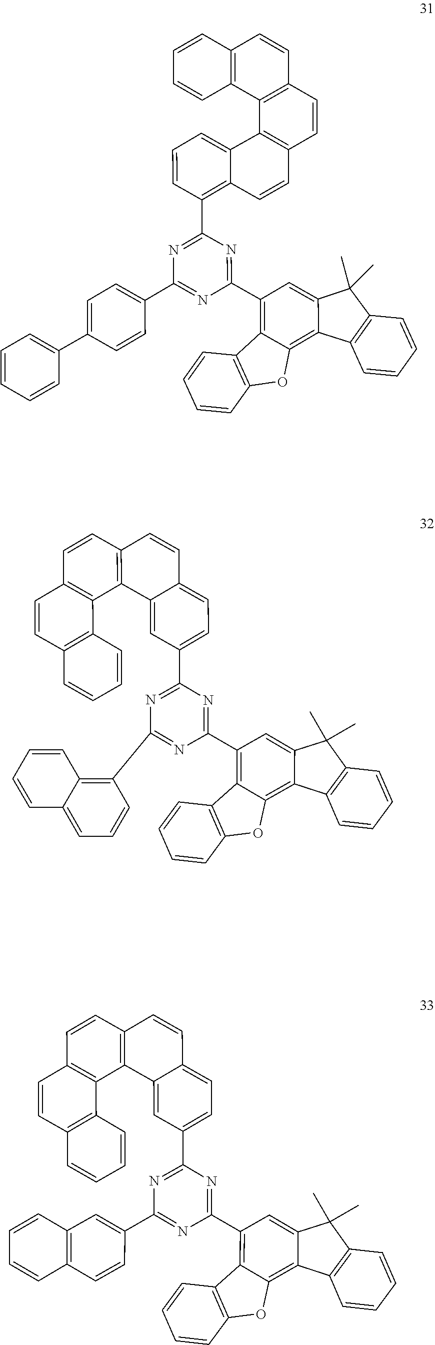 [SMILES: CC1(C)c2ccccc2-c2c1cc(-c1nc(-c3ccc(-c4ccccc4)cc3)nc(-c3cccc4c3ccc3ccc5ccc6ccccc6c5c34)n1)c1c2oc2ccccc21 ; CC1(C)c2ccccc2-c2c1cc(-c1nc(-c3ccc4ccc5ccc6ccc7ccccc7c6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6ccc7ccccc7c6c5c4c3)n1)c1c2oc2ccccc21]

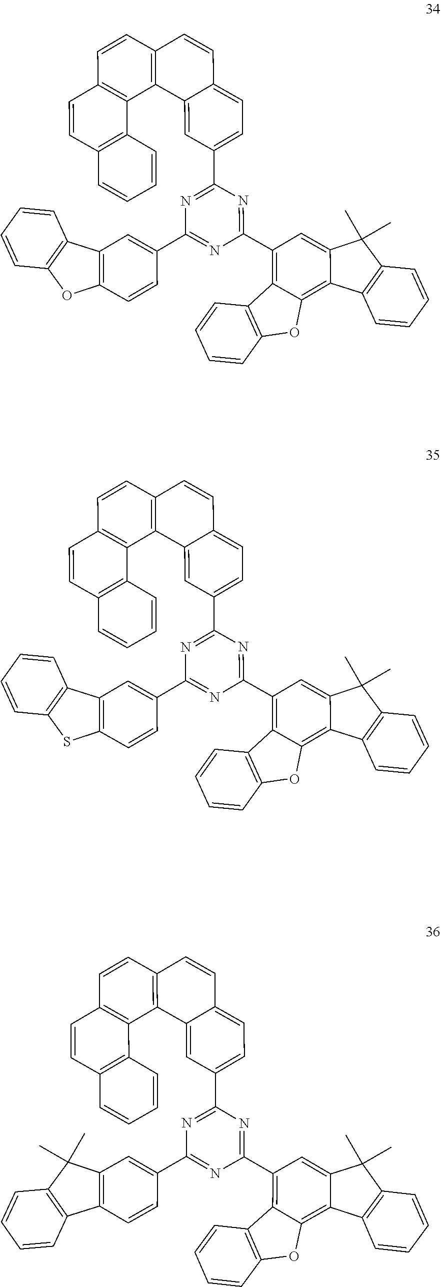 [SMILES: CC1(C)c2ccccc2-c2c1cc(-c1nc(-c3ccc4oc5ccccc5c4c3)nc(-c3ccc4ccc5ccc6ccc7ccccc7c6c5c4c3)n1)c1c2oc2ccccc21 ; CC1(C)c2ccccc2-c2c1cc(-c1nc(-c3ccc4sc5ccccc5c4c3)nc(-c3ccc4ccc5ccc6ccc7ccccc7c6c5c4c3)n1)c1c2oc2ccccc21 ; CC1(C)c2ccccc2-c2ccc(-c3nc(-c4ccc5ccc6ccc7ccc8ccccc8c7c6c5c4)nc(-c4cc5c(c6oc7ccccc7c46)-c4ccccc4C5(C)C)n3)cc21]

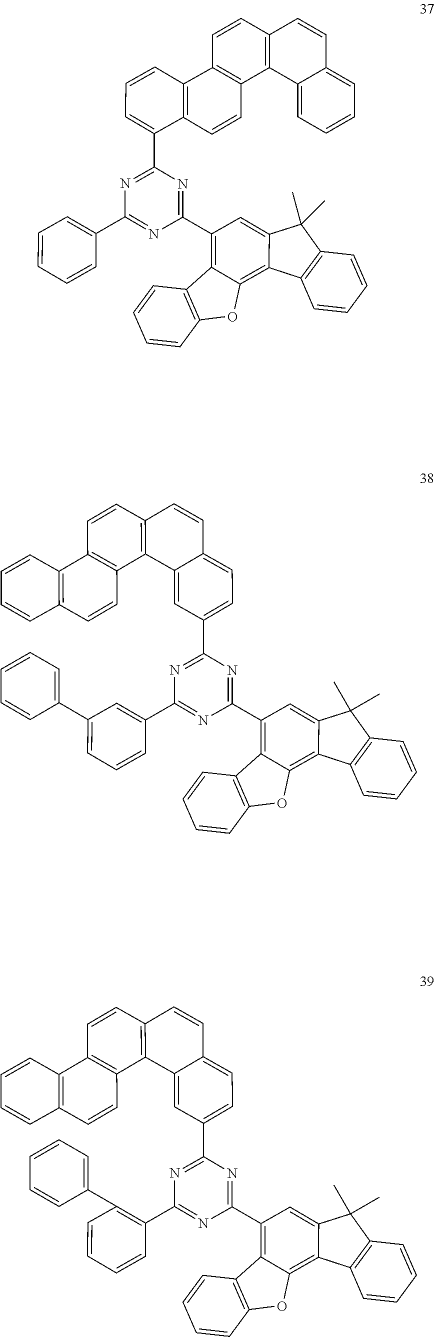 [SMILES: CC1(C)c2ccccc2-c2c1cc(-c1nc(-c3ccccc3)nc(-c3cccc4c3ccc3c4ccc4ccc5ccccc5c43)n1)c1c2oc2ccccc21 ; CC1(C)c2ccccc2-c2c1cc(-c1nc(-c3cccc(-c4ccccc4)c3)nc(-c3ccc4ccc5ccc6c7ccccc7ccc6c5c4c3)n1)c1c2oc2ccccc21 ; CC1(C)c2ccccc2-c2c1cc(-c1nc(-c3ccc4ccc5ccc6c7ccccc7ccc6c5c4c3)nc(-c3ccccc3-c3ccccc3)n1)c1c2oc2ccccc21]

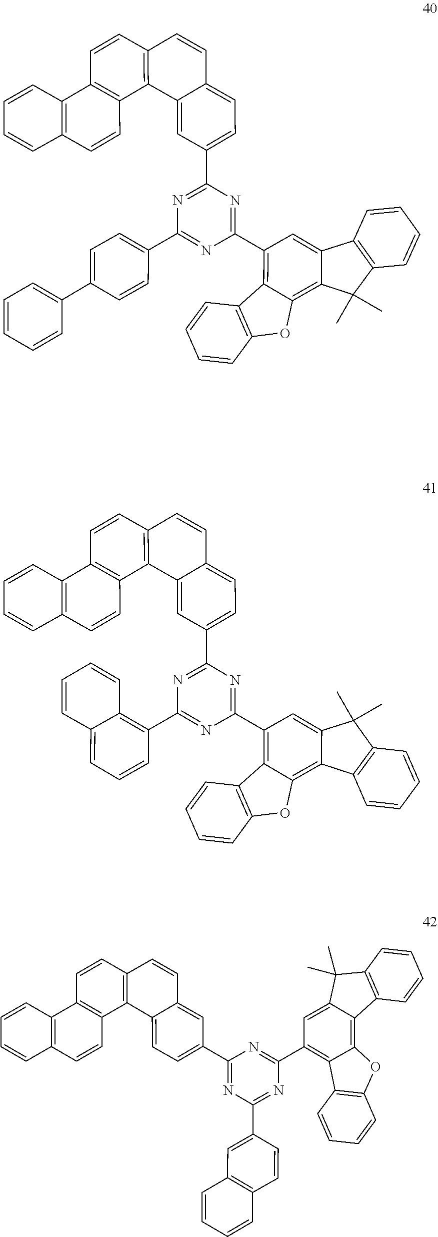 [SMILES: CC1(C)c2ccccc2-c2cc(-c3nc(-c4ccc(-c5ccccc5)cc4)nc(-c4ccc5ccc6ccc7c8ccccc8ccc7c6c5c4)n3)c3c(oc4ccccc43)c21 ; CC1(C)c2ccccc2-c2c1cc(-c1nc(-c3ccc4ccc5ccc6c7ccccc7ccc6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4c(ccc5ccc6c7ccccc7ccc6c54)c3)n1)c1c2oc2ccccc21]

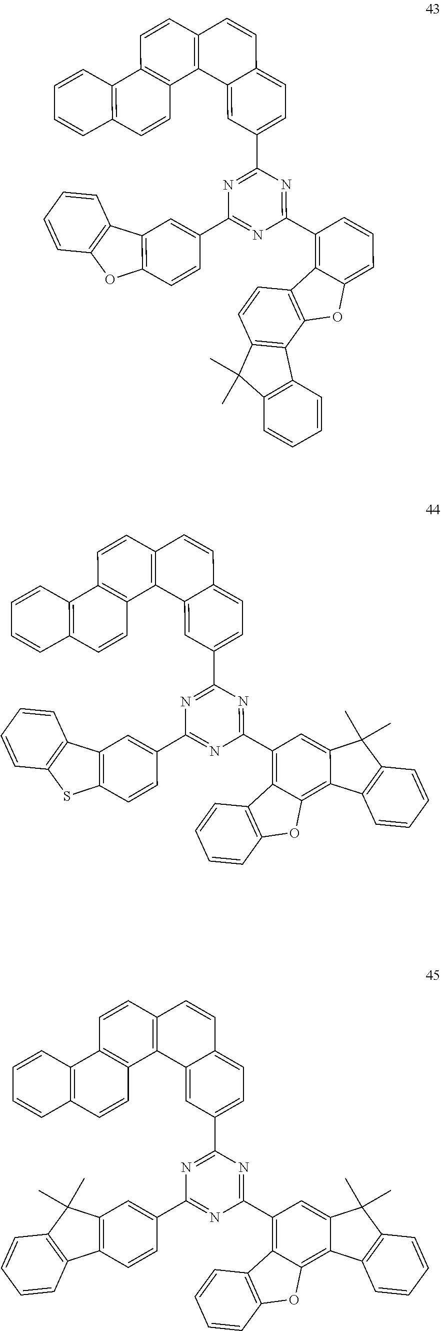 [SMILES: CC1(C)c2ccccc2-c2c1ccc1c2oc2cccc(-c3nc(-c4ccc5oc6ccccc6c5c4)nc(-c4ccc5ccc6ccc7c8ccccc8ccc7c6c5c4)n3)c21 ; CC1(C)c2ccccc2-c2c1cc(-c1nc(-c3ccc4sc5ccccc5c4c3)nc(-c3ccc4ccc5ccc6c7ccccc7ccc6c5c4c3)n1)c1c2oc2ccccc21 ; CC1(C)c2ccccc2-c2ccc(-c3nc(-c4ccc5ccc6ccc7c8ccccc8ccc7c6c5c4)nc(-c4cc5c(c6oc7ccccc7c46)-c4ccccc4C5(C)C)n3)cc21]

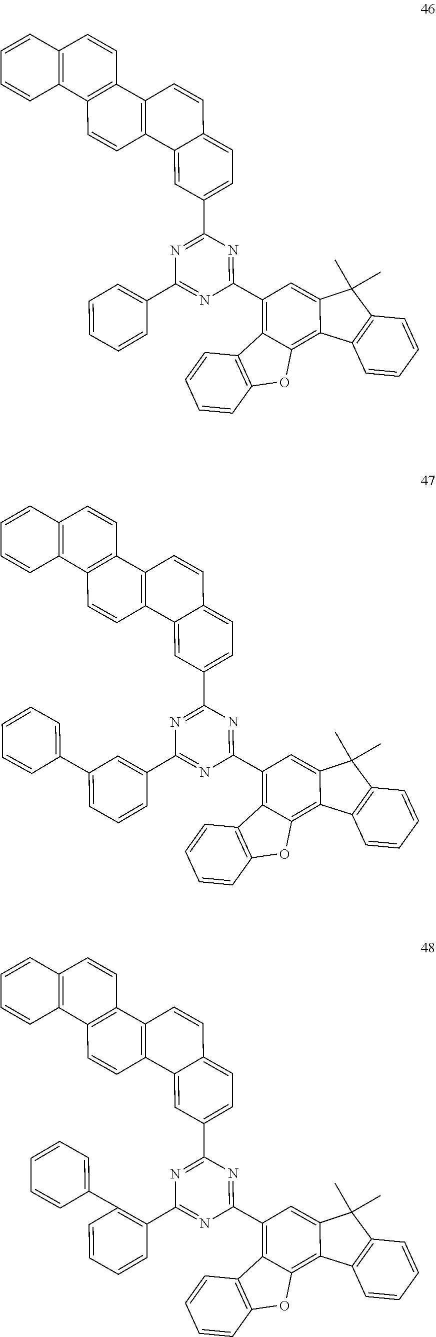 [SMILES: CC1(C)c2ccccc2-c2c1cc(-c1nc(-c3ccccc3)nc(-c3ccc4ccc5c(ccc6c7ccccc7ccc65)c4c3)n1)c1c2oc2ccccc21 ; CC1(C)c2ccccc2-c2c1cc(-c1nc(-c3cccc(-c4ccccc4)c3)nc(-c3ccc4ccc5c(ccc6c7ccccc7ccc65)c4c3)n1)c1c2oc2ccccc21 ; CC1(C)c2ccccc2-c2c1cc(-c1nc(-c3ccc4ccc5c(ccc6c7ccccc7ccc65)c4c3)nc(-c3ccccc3-c3ccccc3)n1)c1c2oc2ccccc21]

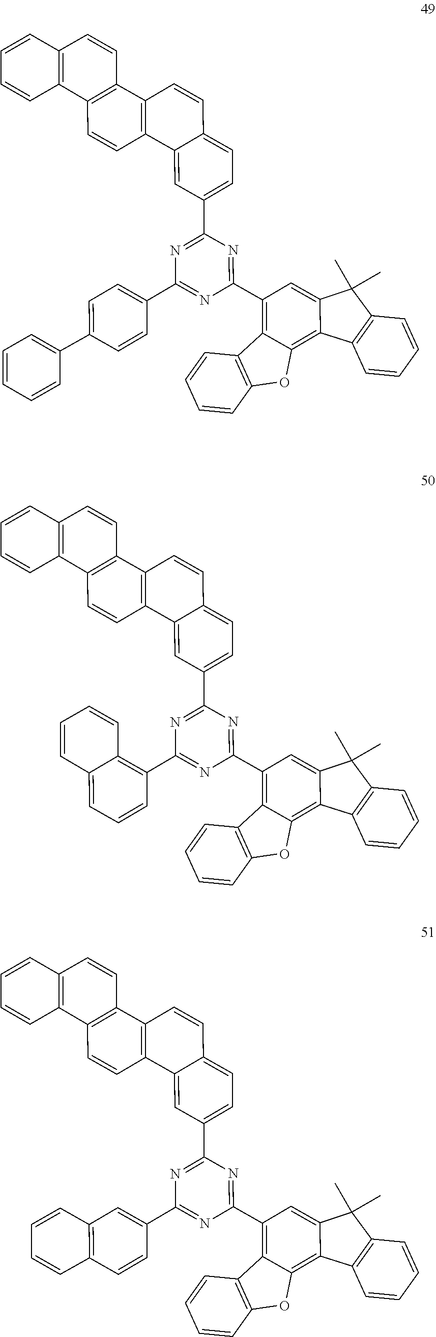 [SMILES: CC1(C)c2ccccc2-c2c1cc(-c1nc(-c3ccc(-c4ccccc4)cc3)nc(-c3ccc4ccc5c(ccc6c7ccccc7ccc65)c4c3)n1)c1c2oc2ccccc21 ; CC1(C)c2ccccc2-c2c1cc(-c1nc(-c3ccc4ccc5c(ccc6c7ccccc7ccc65)c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5c(ccc6c7ccccc7ccc65)c4c3)n1)c1c2oc2ccccc21]

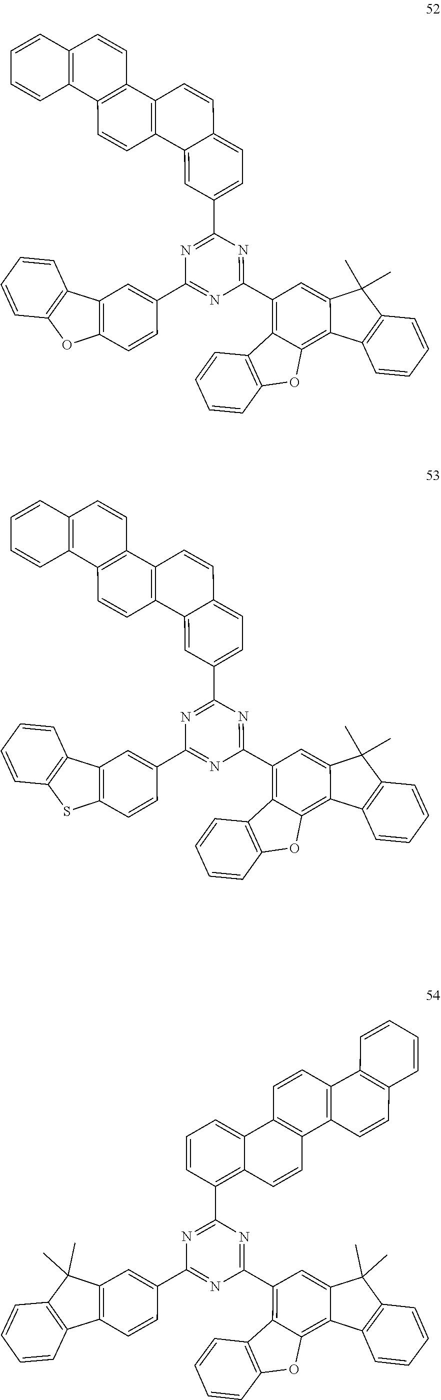 [SMILES: CC1(C)c2ccccc2-c2c1cc(-c1nc(-c3ccc4ccc5c(ccc6c7ccccc7ccc65)c4c3)nc(-c3ccc4oc5ccccc5c4c3)n1)c1c2oc2ccccc21 ; CC1(C)c2ccccc2-c2c1cc(-c1nc(-c3ccc4ccc5c(ccc6c7ccccc7ccc65)c4c3)nc(-c3ccc4sc5ccccc5c4c3)n1)c1c2oc2ccccc21 ; CC1(C)c2ccccc2-c2ccc(-c3nc(-c4cccc5c4ccc4c5ccc5c6ccccc6ccc54)nc(-c4cc5c(c6oc7ccccc7c46)-c4ccccc4C5(C)C)n3)cc21]

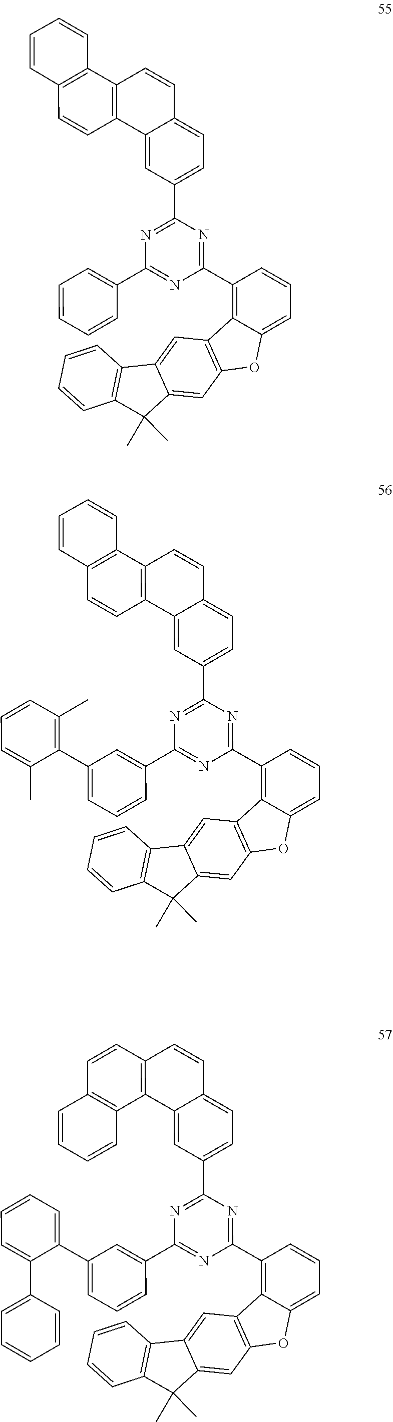 [SMILES: CC1(C)c2ccccc2-c2cc3c(cc21)oc1cccc(-c2nc(-c4ccccc4)nc(-c4ccc5ccc6c7ccccc7ccc6c5c4)n2)c13 ; Cc1cccc(C)c1-c1cccc(-c2nc(-c3ccc4ccc5c6ccccc6ccc5c4c3)nc(-c3cccc4oc5cc6c(cc5c34)-c3ccccc3C6(C)C)n2)c1 ; CC1(C)c2ccccc2-c2cc3c(cc21)oc1cccc(-c2nc(-c4cccc(-c5ccccc5-c5ccccc5)c4)nc(-c4ccc5ccc6ccc7ccccc7c6c5c4)n2)c13]

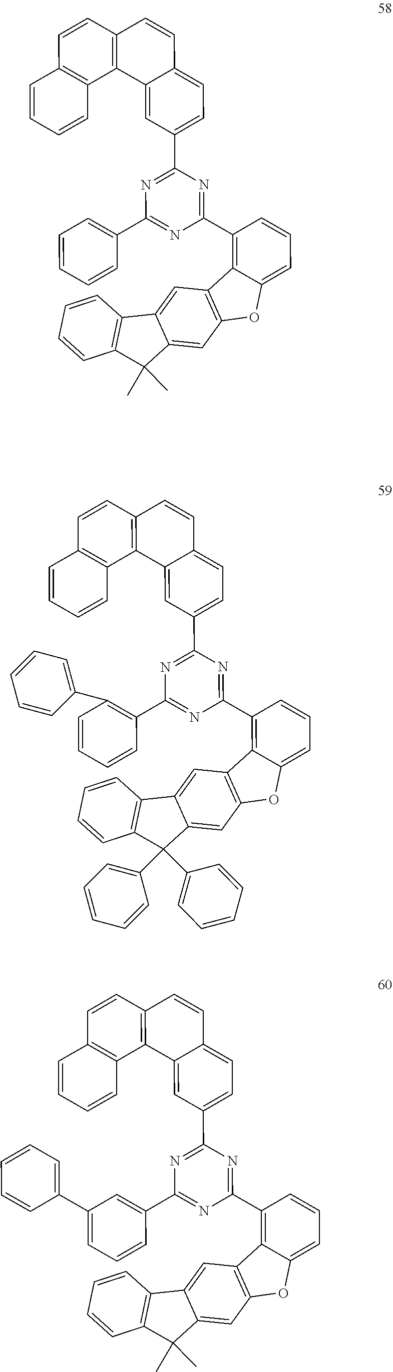 [SMILES: CC1(C)c2ccccc2-c2cc3c(cc21)oc1cccc(-c2nc(-c4ccccc4)nc(-c4ccc5ccc6ccc7ccccc7c6c5c4)n2)c13 ; c1ccc(-c2ccccc2-c2nc(-c3ccc4ccc5ccc6ccccc6c5c4c3)nc(-c3cccc4oc5cc6c(cc5c34)-c3ccccc3C6(c3ccccc3)c3ccccc3)n2)cc1 ; CC1(C)c2ccccc2-c2cc3c(cc21)oc1cccc(-c2nc(-c4cccc(-c5ccccc5)c4)nc(-c4ccc5ccc6ccc7ccccc7c6c5c4)n2)c13]

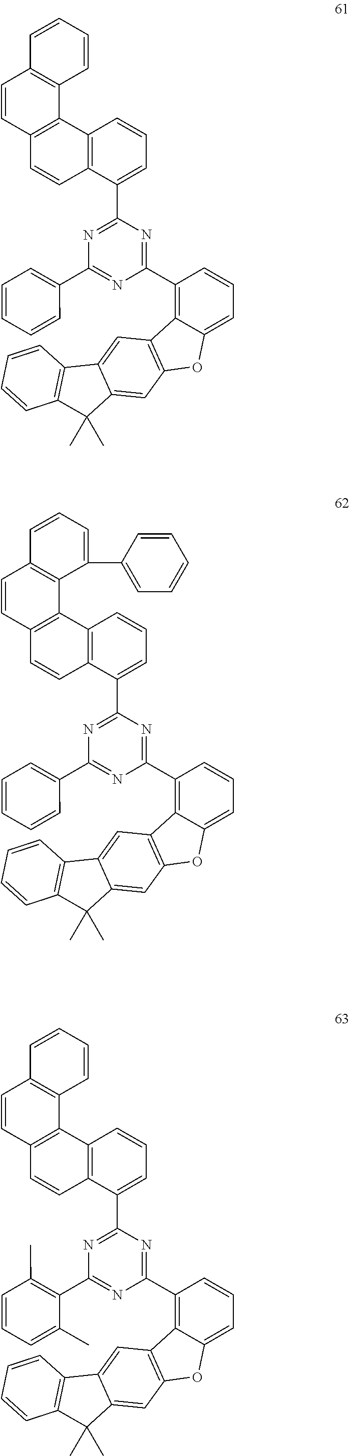 [SMILES: CC1(C)c2ccccc2-c2cc3c(cc21)oc1cccc(-c2nc(-c4ccccc4)nc(-c4cccc5c4ccc4ccc6ccccc6c45)n2)c13 ; CC1(C)c2ccccc2-c2cc3c(cc21)oc1cccc(-c2nc(-c4ccccc4)nc(-c4cccc5c4ccc4ccc6cccc(-c7ccccc7)c6c45)n2)c13 ; Cc1cccc(C)c1-c1nc(-c2cccc3c2ccc2ccc4ccccc4c23)nc(-c2cccc3oc4cc5c(cc4c23)-c2ccccc2C5(C)C)n1]

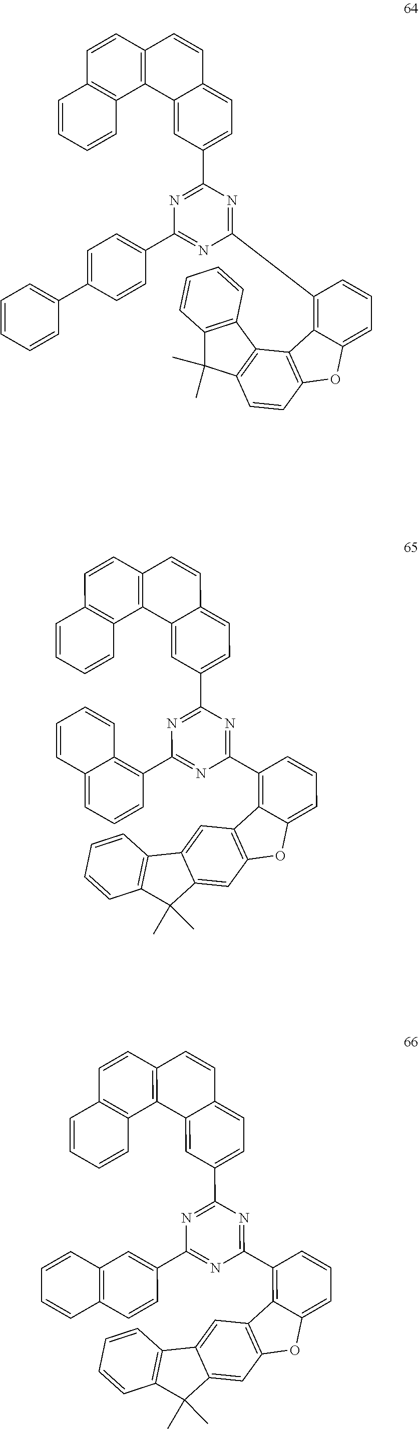 [SMILES: CC1(C)c2ccccc2-c2c1ccc1oc3cccc(-c4nc(-c5ccc(-c6ccccc6)cc5)nc(-c5ccc6ccc7ccc8ccccc8c7c6c5)n4)c3c21 ; CC1(C)c2ccccc2-c2cc3c(cc21)oc1cccc(-c2nc(-c4ccc5ccc6ccc7ccccc7c6c5c4)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6ccc7ccccc7c6c5c4)n2)c13]

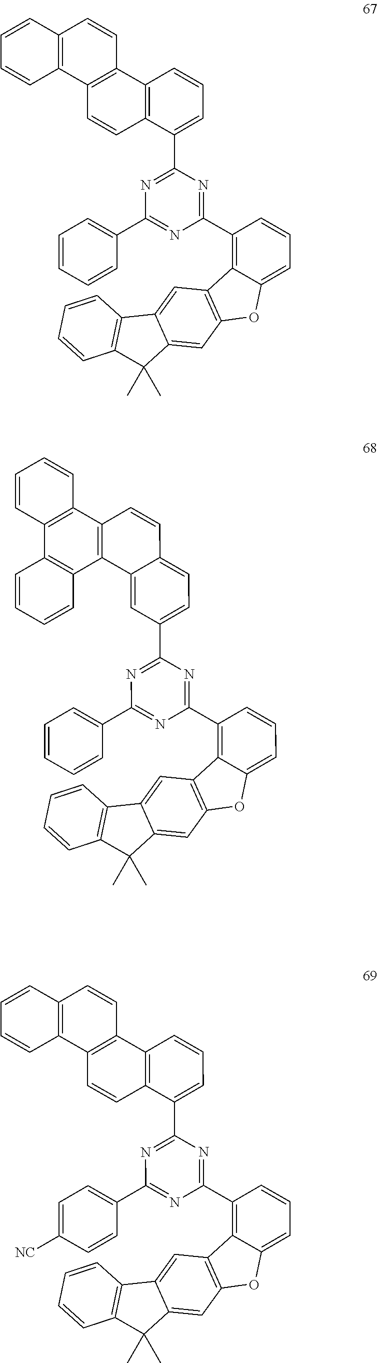 [SMILES: CC1(C)c2ccccc2-c2cc3c(cc21)oc1cccc(-c2nc(-c4ccccc4)nc(-c4cccc5c4ccc4c6ccccc6ccc54)n2)c13 ; CC1(C)c2ccccc2-c2cc3c(cc21)oc1cccc(-c2nc(-c4ccccc4)nc(-c4ccc5ccc6c7ccccc7c7ccccc7c6c5c4)n2)c13 ; CC1(C)c2ccccc2-c2cc3c(cc21)oc1cccc(-c2nc(-c4ccc(C#N)cc4)nc(-c4cccc5c4ccc4c6ccccc6ccc54)n2)c13]

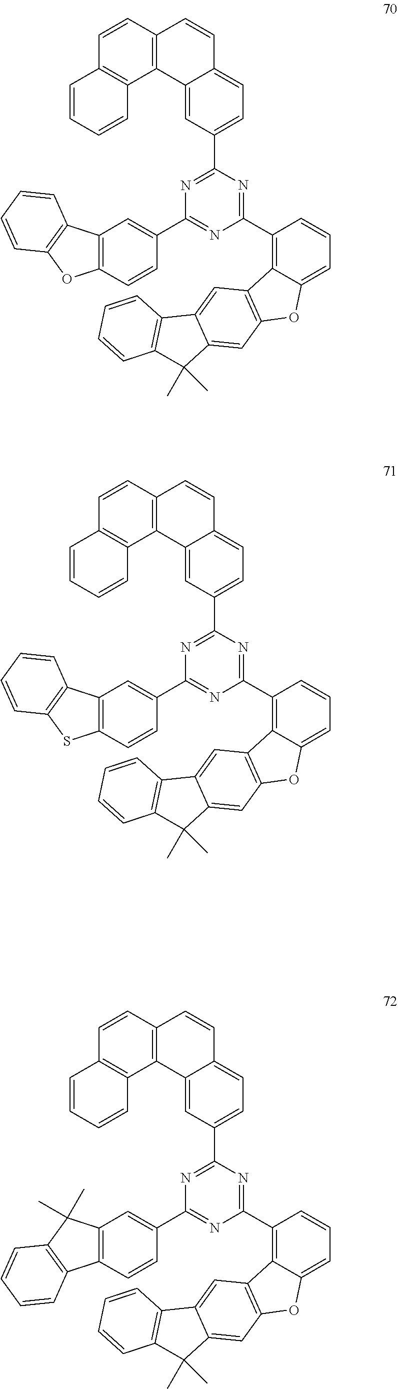 [SMILES: CC1(C)c2ccccc2-c2cc3c(cc21)oc1cccc(-c2nc(-c4ccc5oc6ccccc6c5c4)nc(-c4ccc5ccc6ccc7ccccc7c6c5c4)n2)c13 ; CC1(C)c2ccccc2-c2cc3c(cc21)oc1cccc(-c2nc(-c4ccc5sc6ccccc6c5c4)nc(-c4ccc5ccc6ccc7ccccc7c6c5c4)n2)c13 ; CC1(C)c2ccccc2-c2ccc(-c3nc(-c4ccc5ccc6ccc7ccccc7c6c5c4)nc(-c4cccc5oc6cc7c(cc6c45)-c4ccccc4C7(C)C)n3)cc21]

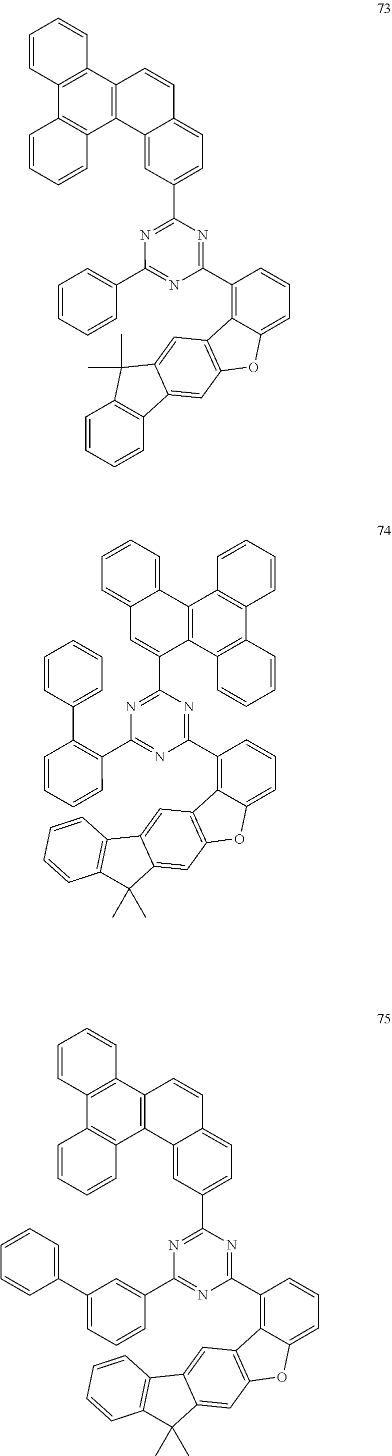 [SMILES: CC1(C)c2ccccc2-c2cc3oc4cccc(-c5nc(-c6ccccc6)nc(-c6ccc7ccc8c9ccccc9c9ccccc9c8c7c6)n5)c4c3cc21 ; CC1(C)c2ccccc2-c2cc3c(cc21)oc1cccc(-c2nc(-c4ccccc4-c4ccccc4)nc(-c4cc5ccccc5c5c6ccccc6c6ccccc6c45)n2)c13 ; CC1(C)c2ccccc2-c2cc3c(cc21)oc1cccc(-c2nc(-c4cccc(-c5ccccc5)c4)nc(-c4ccc5ccc6c7ccccc7c7ccccc7c6c5c4)n2)c13]

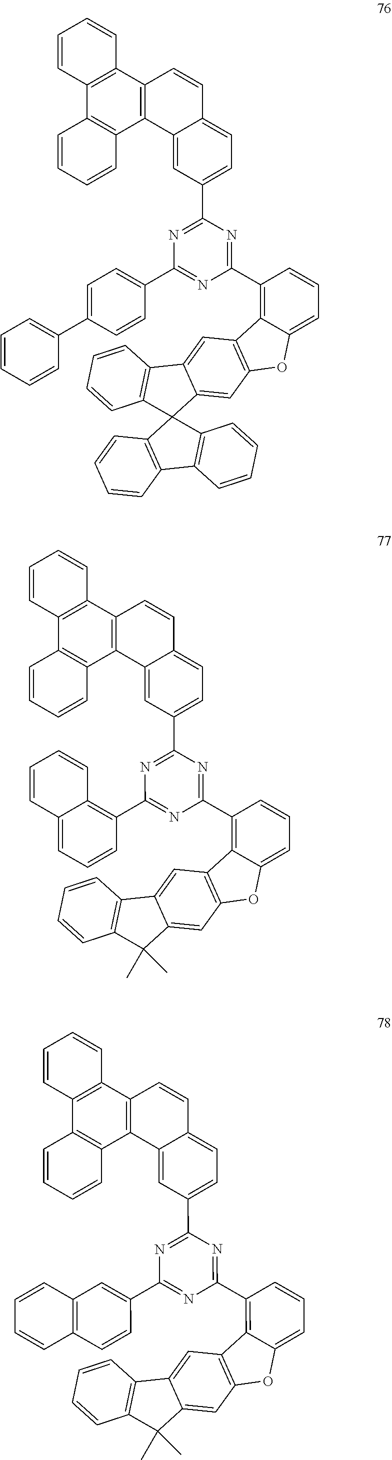 [SMILES: c1ccc(-c2ccc(-c3nc(-c4ccc5ccc6c7ccccc7c7ccccc7c6c5c4)nc(-c4cccc5oc6cc7c(cc6c45)-c4ccccc4C74c5ccccc5-c5ccccc54)n3)cc2)cc1 ; CC1(C)c2ccccc2-c2cc3c(cc21)oc1cccc(-c2nc(-c4ccc5ccc6c7ccccc7c7ccccc7c6c5c4)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6c7ccccc7c7ccccc7c6c5c4)n2)c13]

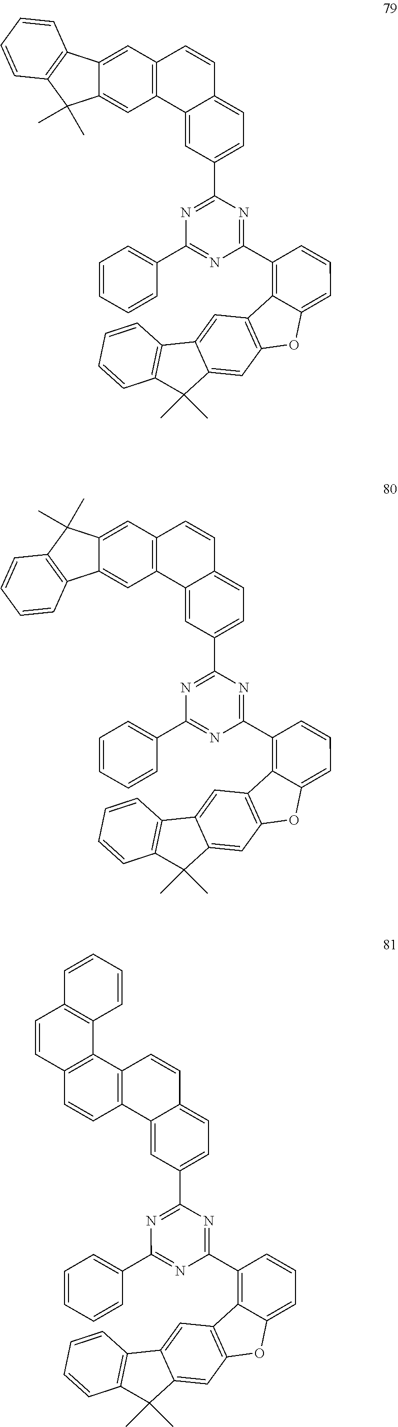 [SMILES: CC1(C)c2ccccc2-c2cc3c(cc21)oc1cccc(-c2nc(-c4ccccc4)nc(-c4ccc5ccc6cc7c(cc6c5c4)C(C)(C)c4ccccc4-7)n2)c13 ; CC1(C)c2ccccc2-c2cc3c(ccc4ccc(-c5nc(-c6ccccc6)nc(-c6cccc7oc8cc9c(cc8c67)-c6ccccc6C9(C)C)n5)cc43)cc21 ; CC1(C)c2ccccc2-c2cc3c(cc21)oc1cccc(-c2nc(-c4ccccc4)nc(-c4ccc5ccc6c(ccc7ccc8ccccc8c76)c5c4)n2)c13]

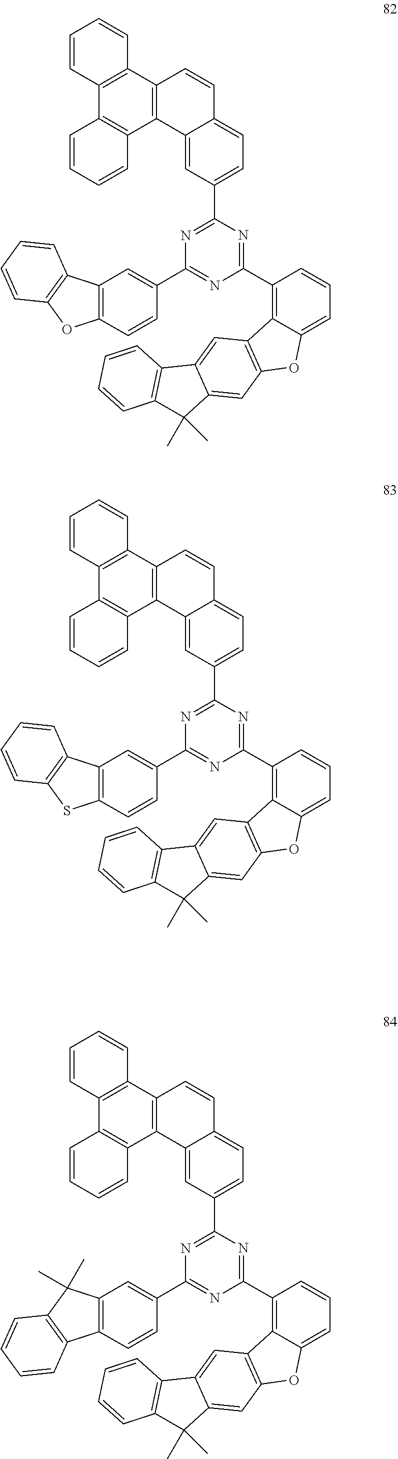 [SMILES: CC1(C)c2ccccc2-c2cc3c(cc21)oc1cccc(-c2nc(-c4ccc5oc6ccccc6c5c4)nc(-c4ccc5ccc6c7ccccc7c7ccccc7c6c5c4)n2)c13 ; CC1(C)c2ccccc2-c2cc3c(cc21)oc1cccc(-c2nc(-c4ccc5sc6ccccc6c5c4)nc(-c4ccc5ccc6c7ccccc7c7ccccc7c6c5c4)n2)c13 ; CC1(C)c2ccccc2-c2ccc(-c3nc(-c4ccc5ccc6c7ccccc7c7ccccc7c6c5c4)nc(-c4cccc5oc6cc7c(cc6c45)-c4ccccc4C7(C)C)n3)cc21]

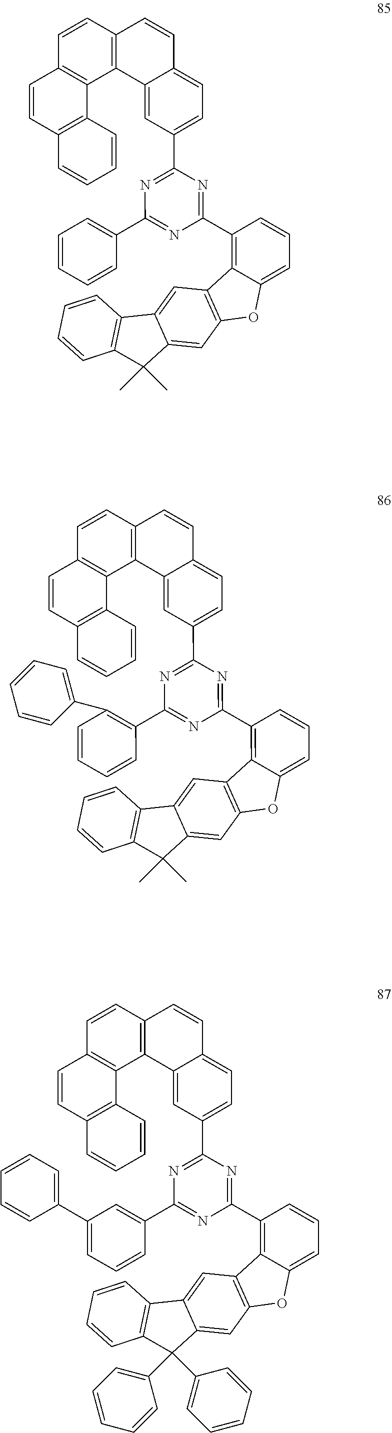 [SMILES: CC1(C)c2ccccc2-c2cc3c(cc21)oc1cccc(-c2nc(-c4ccccc4)nc(-c4ccc5ccc6ccc7ccc8ccccc8c7c6c5c4)n2)c13 ; CC1(C)c2ccccc2-c2cc3c(cc21)oc1cccc(-c2nc(-c4ccc5ccc6ccc7ccc8ccccc8c7c6c5c4)nc(-c4ccccc4-c4ccccc4)n2)c13 ; c1ccc(-c2cccc(-c3nc(-c4ccc5ccc6ccc7ccc8ccccc8c7c6c5c4)nc(-c4cccc5oc6cc7c(cc6c45)-c4ccccc4C7(c4ccccc4)c4ccccc4)n3)c2)cc1]

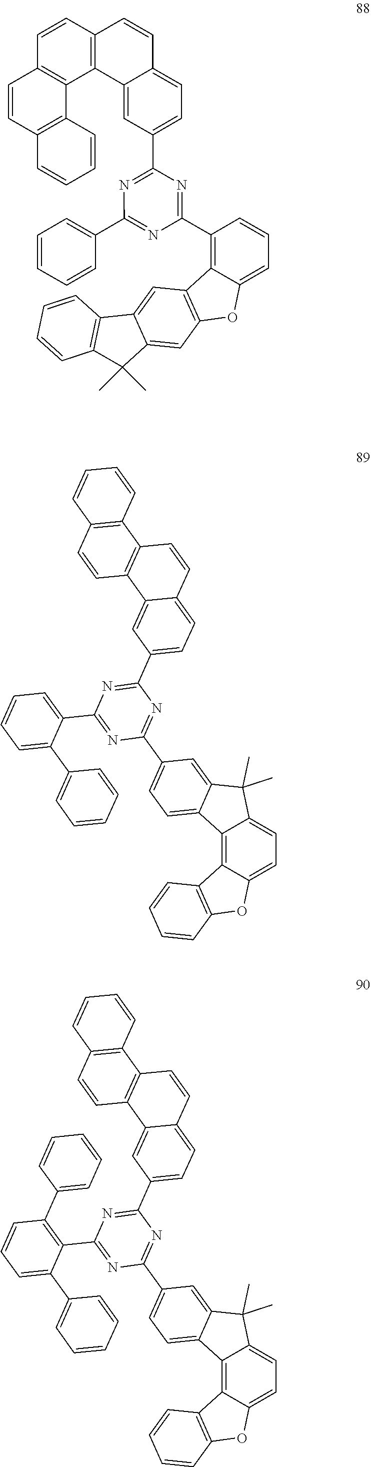 [SMILES: CC1(C)c2ccccc2-c2cc3c(cc21)oc1cccc(-c2nc(-c4ccccc4)nc(-c4ccc5ccc6ccc7ccc8ccccc8c7c6c5c4)n2)c13 ; CC1(C)c2cc(-c3nc(-c4ccc5ccc6c7ccccc7ccc6c5c4)nc(-c4ccccc4-c4ccccc4)n3)ccc2-c2c1ccc1oc3ccccc3c21 ; CC1(C)c2cc(-c3nc(-c4ccc5ccc6c7ccccc7ccc6c5c4)nc(-c4c(-c5ccccc5)cccc4-c4ccccc4)n3)ccc2-c2c1ccc1oc3ccccc3c21]

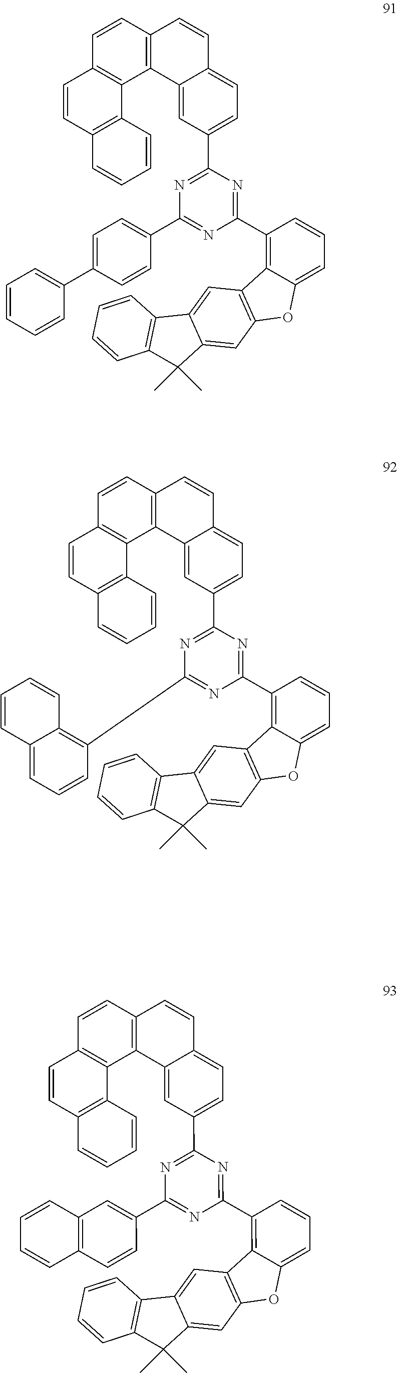 [SMILES: CC1(C)c2ccccc2-c2cc3c(cc21)oc1cccc(-c2nc(-c4ccc(-c5ccccc5)cc4)nc(-c4ccc5ccc6ccc7ccc8ccccc8c7c6c5c4)n2)c13 ; CC1(C)c2ccccc2-c2cc3c(cc21)oc1cccc(-c2nc(-c4ccc5ccc6ccc7ccc8ccccc8c7c6c5c4)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6ccc7ccc8ccccc8c7c6c5c4)n2)c13]

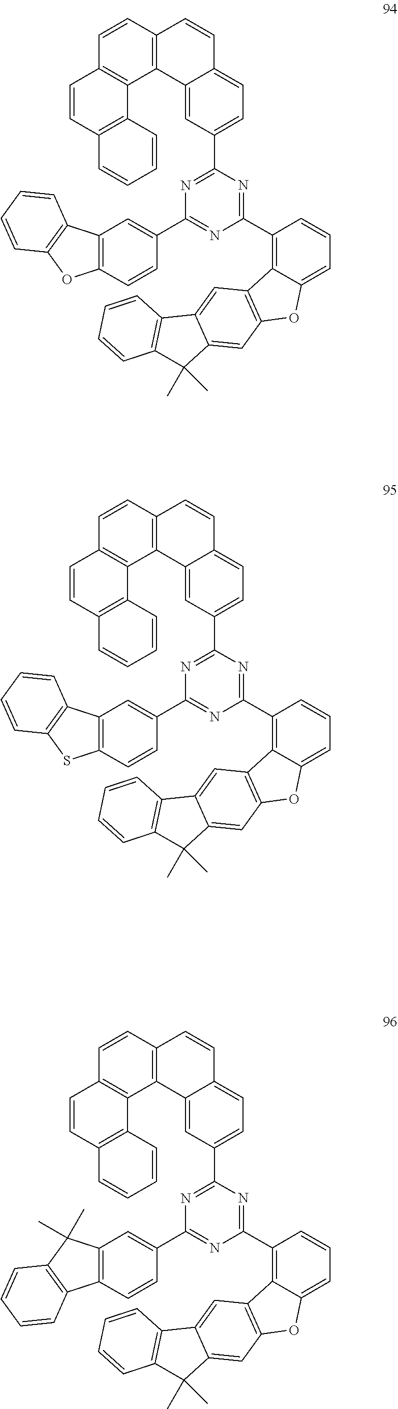 [SMILES: CC1(C)c2ccccc2-c2cc3c(cc21)oc1cccc(-c2nc(-c4ccc5oc6ccccc6c5c4)nc(-c4ccc5ccc6ccc7ccc8ccccc8c7c6c5c4)n2)c13 ; CC1(C)c2ccccc2-c2cc3c(cc21)oc1cccc(-c2nc(-c4ccc5sc6ccccc6c5c4)nc(-c4ccc5ccc6ccc7ccc8ccccc8c7c6c5c4)n2)c13 ; CC1(C)c2ccccc2-c2ccc(-c3nc(-c4ccc5ccc6ccc7ccc8ccccc8c7c6c5c4)nc(-c4cccc5oc6cc7c(cc6c45)-c4ccccc4C7(C)C)n3)cc21]

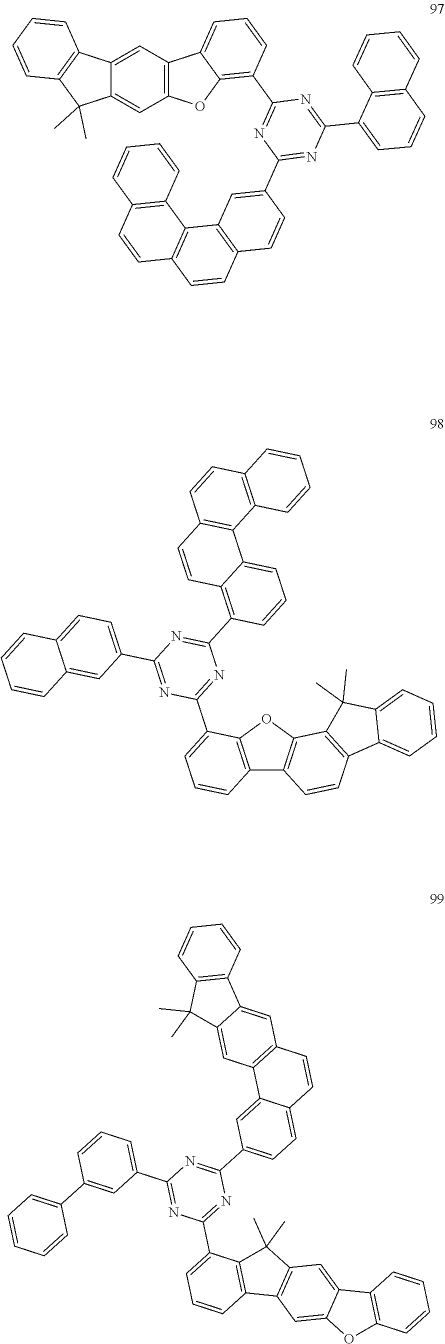 [SMILES: CC1(C)c2ccccc2-c2cc3c(cc21)oc1c(-c2nc(-c4ccc5ccc6ccc7ccccc7c6c5c4)nc(-c4cccc5ccccc45)n2)cccc13 ; CC1(C)c2ccccc2-c2ccc3c(oc4c(-c5nc(-c6ccc7ccccc7c6)nc(-c6cccc7c6ccc6ccc8ccccc8c67)n5)cccc43)c21 ; CC1(C)c2ccccc2-c2cc3ccc4ccc(-c5nc(-c6cccc(-c7ccccc7)c6)nc(-c6cccc7c6C(C)(C)c6cc8c(cc6-7)oc6ccccc68)n5)cc4c3cc21]

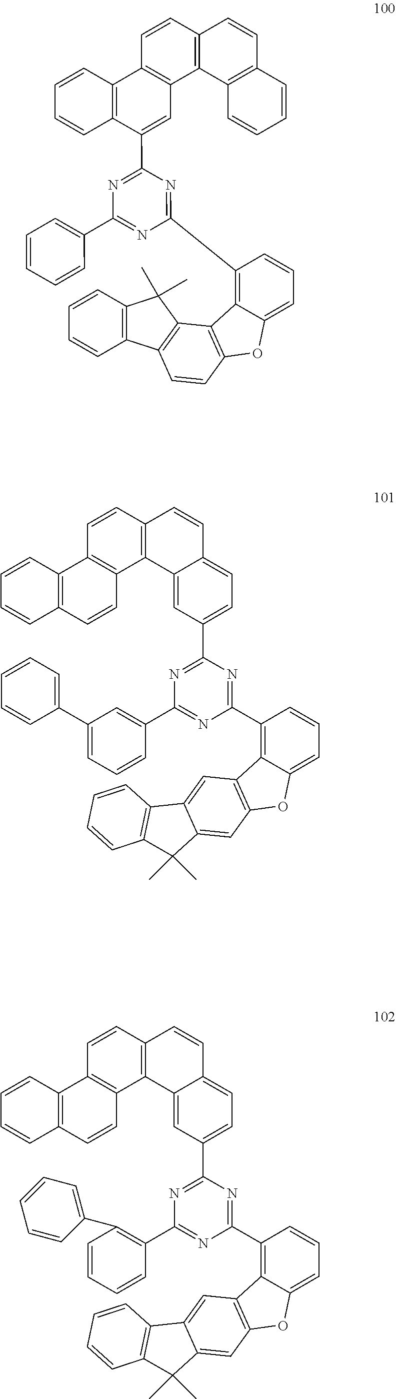 [SMILES: CC1(C)c2ccccc2-c2ccc3oc4cccc(-c5nc(-c6ccccc6)nc(-c6cc7c(ccc8ccc9ccccc9c87)c7ccccc67)n5)c4c3c21 ; CC1(C)c2ccccc2-c2cc3c(cc21)oc1cccc(-c2nc(-c4cccc(-c5ccccc5)c4)nc(-c4ccc5ccc6ccc7c8ccccc8ccc7c6c5c4)n2)c13 ; CC1(C)c2ccccc2-c2cc3c(cc21)oc1cccc(-c2nc(-c4ccc5ccc6ccc7c8ccccc8ccc7c6c5c4)nc(-c4ccccc4-c4ccccc4)n2)c13]

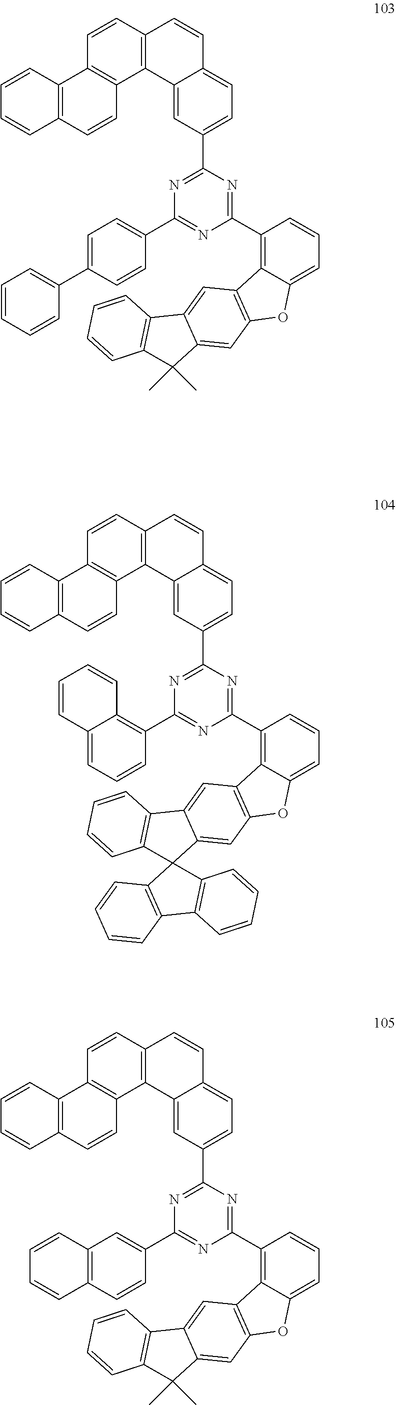

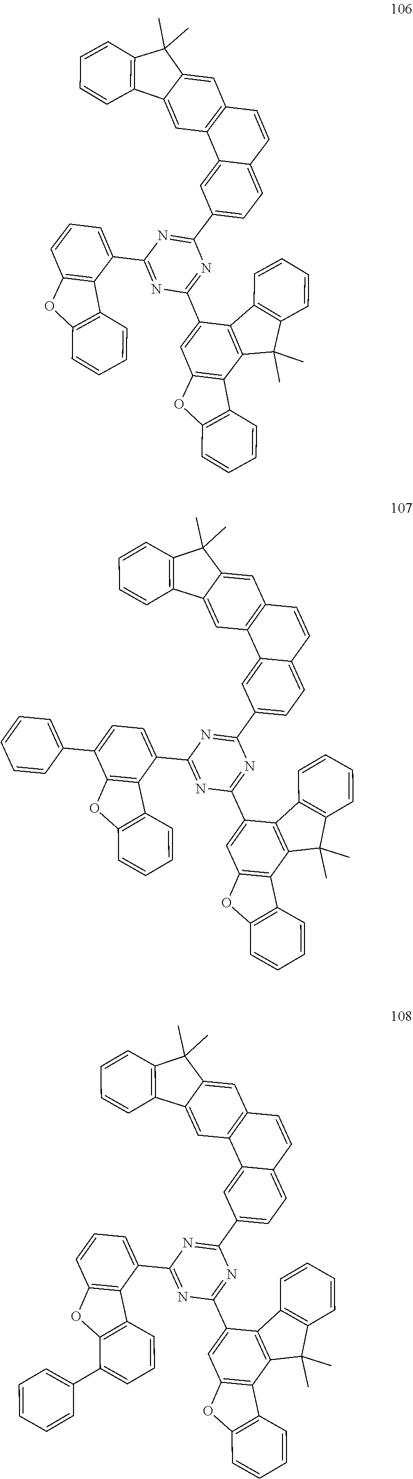 [SMILES: CC1(C)c2ccccc2-c2cc3c(ccc4ccc(-c5nc(-c6cc7oc8ccccc8c7c7c6-c6ccccc6C7(C)C)nc(-c6cccc7oc8ccccc8c67)n5)cc43)cc21 ; CC1(C)c2ccccc2-c2cc3c(ccc4ccc(-c5nc(-c6cc7oc8ccccc8c7c7c6-c6ccccc6C7(C)C)nc(-c6ccc(-c7ccccc7)c7oc8ccccc8c67)n5)cc43)cc21 ; CC1(C)c2ccccc2-c2cc3c(ccc4ccc(-c5nc(-c6cc7oc8ccccc8c7c7c6-c6ccccc6C7(C)C)nc(-c6cccc7oc8c(-c9ccccc9)cccc8c67)n5)cc43)cc21]

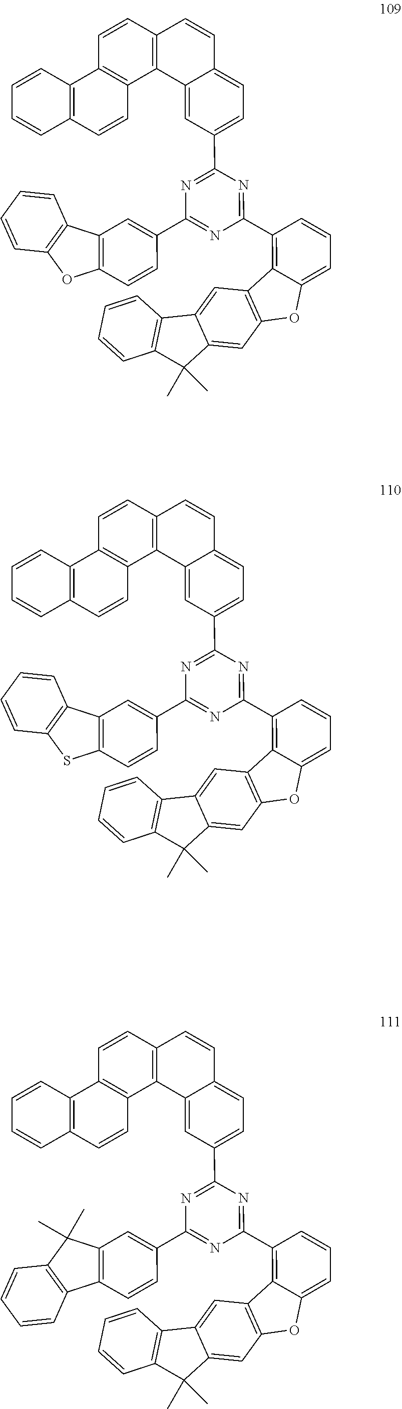 [SMILES: CC1(C)c2ccccc2-c2cc3c(cc21)oc1cccc(-c2nc(-c4ccc5oc6ccccc6c5c4)nc(-c4ccc5ccc6ccc7c8ccccc8ccc7c6c5c4)n2)c13 ; CC1(C)c2ccccc2-c2cc3c(cc21)oc1cccc(-c2nc(-c4ccc5sc6ccccc6c5c4)nc(-c4ccc5ccc6ccc7c8ccccc8ccc7c6c5c4)n2)c13 ; CC1(C)c2ccccc2-c2ccc(-c3nc(-c4ccc5ccc6ccc7c8ccccc8ccc7c6c5c4)nc(-c4cccc5oc6cc7c(cc6c45)-c4ccccc4C7(C)C)n3)cc21]

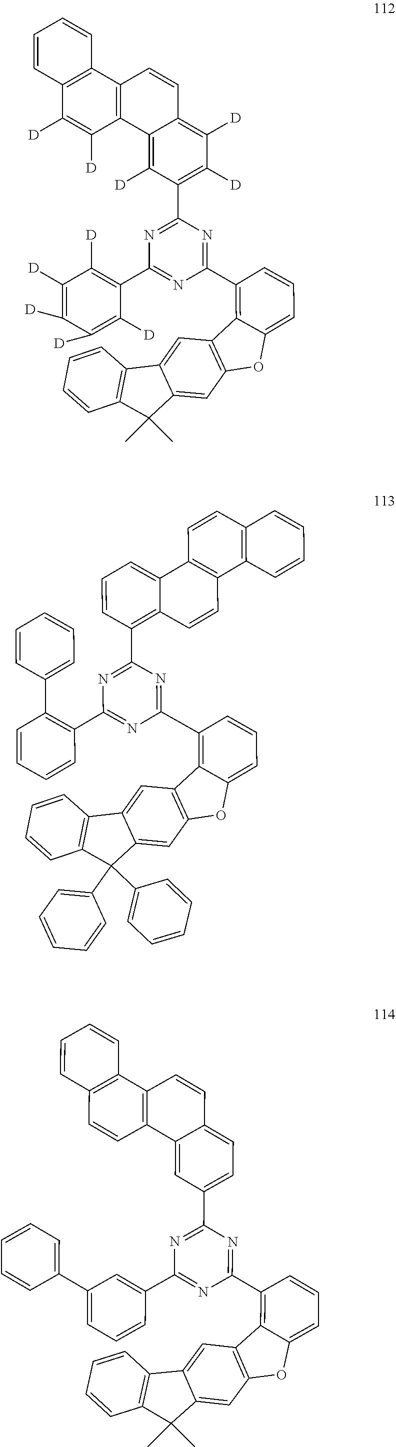 [SMILES: [2H]c1c([2H])c([2H])c(-c2nc(-c3c([2H])c([2H])c4ccc5c6ccccc6c([2H])c([2H])c5c4c3[2H])nc(-c3cccc4oc5cc6c(cc5c34)-c3ccccc3C6(C)C)n2)c([2H])c1[2H] ; c1ccc(-c2ccccc2-c2nc(-c3cccc4c3ccc3c5ccccc5ccc43)nc(-c3cccc4oc5cc6c(cc5c34)-c3ccccc3C6(c3ccccc3)c3ccccc3)n2)cc1 ; CC1(C)c2ccccc2-c2cc3c(cc21)oc1cccc(-c2nc(-c4cccc(-c5ccccc5)c4)nc(-c4ccc5ccc6c7ccccc7ccc6c5c4)n2)c13]

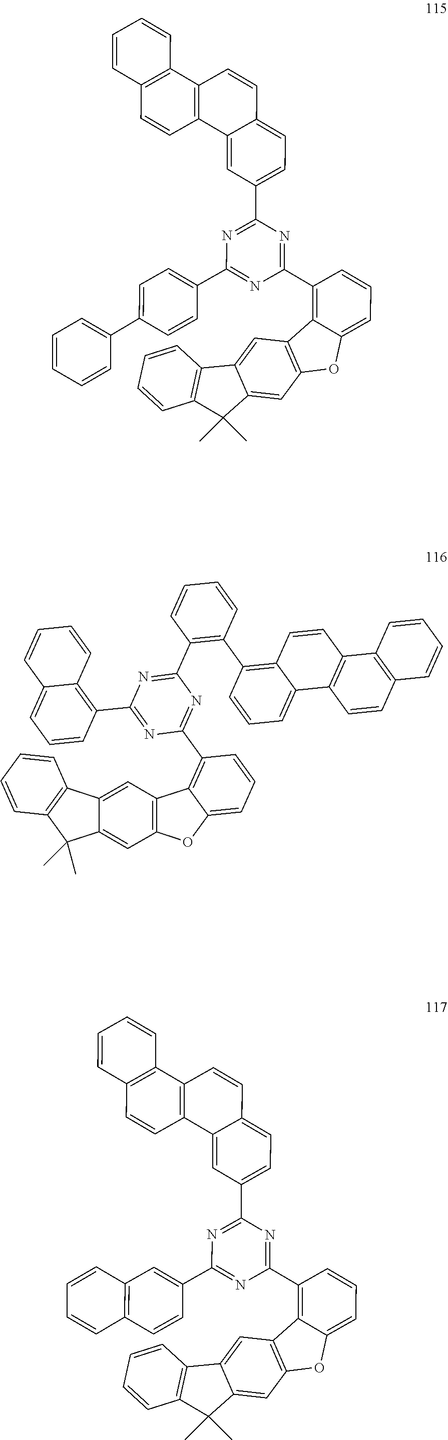 [SMILES: CC1(C)c2ccccc2-c2cc3c(cc21)oc1cccc(-c2nc(-c4ccc(-c5ccccc5)cc4)nc(-c4ccc5ccc6c7ccccc7ccc6c5c4)n2)c13 ; CC1(C)c2ccccc2-c2cc3c(cc21)oc1cccc(-c2nc(-c4ccccc4-c4cccc5c4ccc4c6ccccc6ccc54)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6c7ccccc7ccc6c5c4)n2)c13]

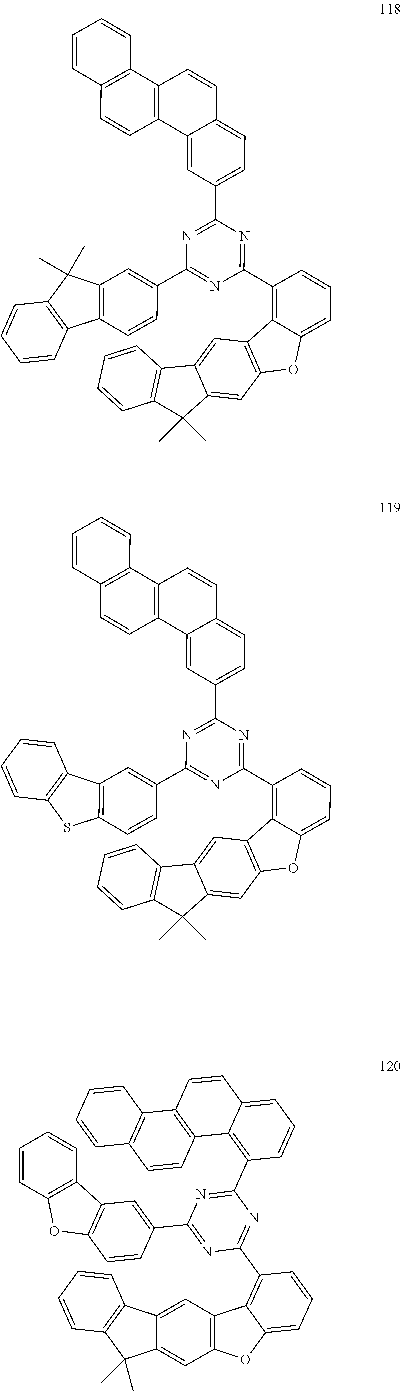 [SMILES: CC1(C)c2ccccc2-c2ccc(-c3nc(-c4ccc5ccc6c7ccccc7ccc6c5c4)nc(-c4cccc5oc6cc7c(cc6c45)-c4ccccc4C7(C)C)n3)cc21 ; CC1(C)c2ccccc2-c2cc3c(cc21)oc1cccc(-c2nc(-c4ccc5ccc6c7ccccc7ccc6c5c4)nc(-c4ccc5sc6ccccc6c5c4)n2)c13 ; CC1(C)c2ccccc2-c2cc3c(cc21)oc1cccc(-c2nc(-c4ccc5oc6ccccc6c5c4)nc(-c4cccc5ccc6c7ccccc7ccc6c45)n2)c13]

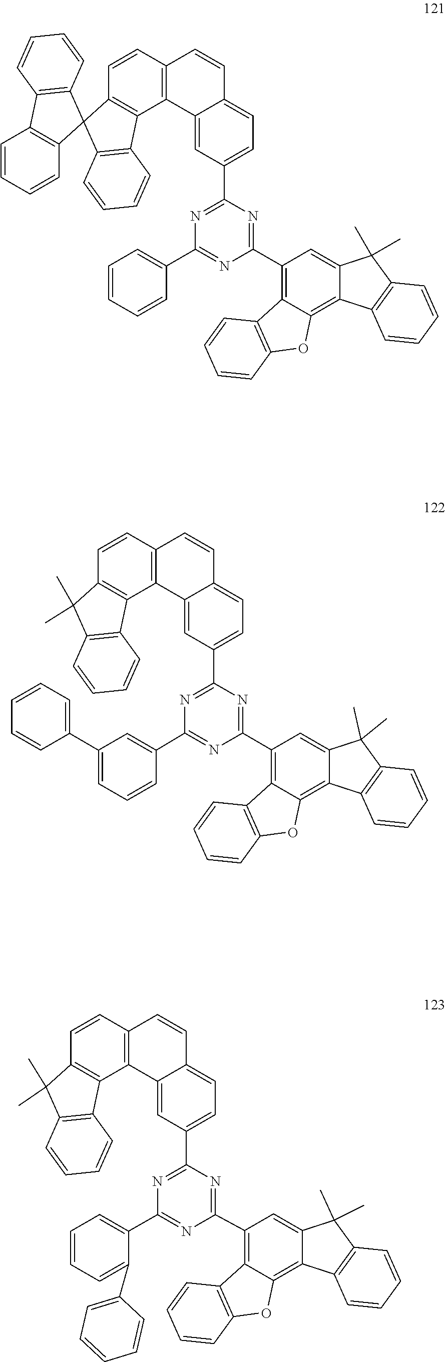 [SMILES: CC1(C)c2ccccc2-c2c1cc(-c1nc(-c3ccccc3)nc(-c3ccc4ccc5ccc6c(c5c4c3)-c3ccccc3C63c4ccccc4-c4ccccc43)n1)c1c2oc2ccccc21 ; CC1(C)c2ccccc2-c2c1cc(-c1nc(-c3cccc(-c4ccccc4)c3)nc(-c3ccc4ccc5ccc6c(c5c4c3)-c3ccccc3C6(C)C)n1)c1c2oc2ccccc21 ; CC1(C)c2ccccc2-c2c1cc(-c1nc(-c3ccc4ccc5ccc6c(c5c4c3)-c3ccccc3C6(C)C)nc(-c3ccccc3-c3ccccc3)n1)c1c2oc2ccccc21]

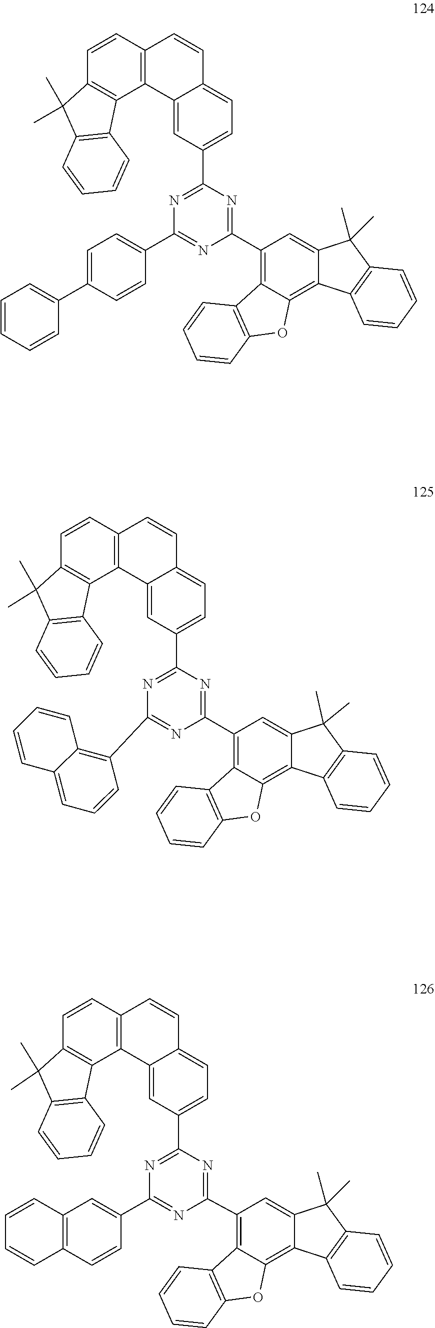 [SMILES: CC1(C)c2ccccc2-c2c1cc(-c1nc(-c3ccc(-c4ccccc4)cc3)nc(-c3ccc4ccc5ccc6c(c5c4c3)-c3ccccc3C6(C)C)n1)c1c2oc2ccccc21 ; CC1(C)c2ccccc2-c2c1cc(-c1nc(-c3ccc4ccc5ccc6c(c5c4c3)-c3ccccc3C6(C)C)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6c(c5c4c3)-c3ccccc3C6(C)C)n1)c1c2oc2ccccc21]

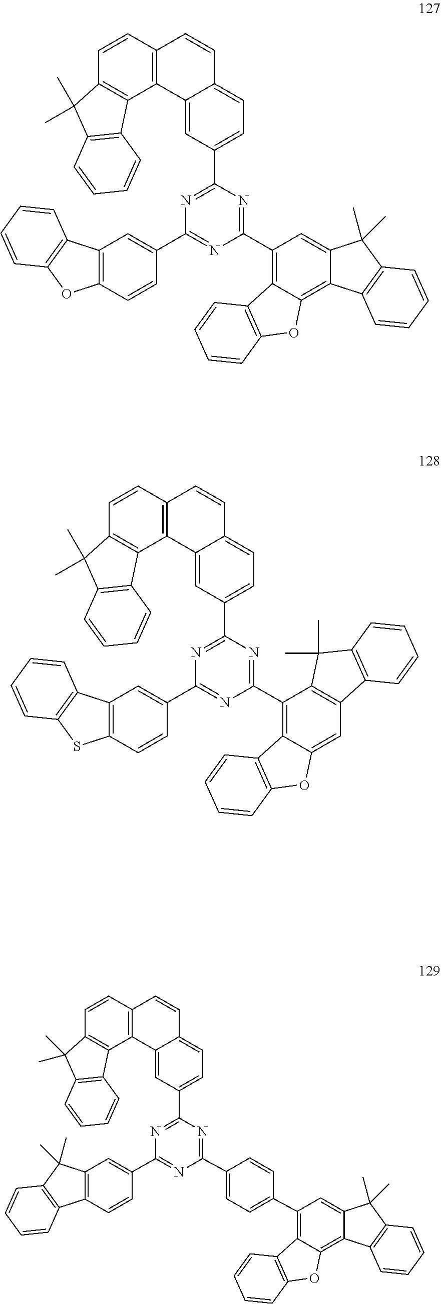 [SMILES: CC1(C)c2ccccc2-c2c1cc(-c1nc(-c3ccc4oc5ccccc5c4c3)nc(-c3ccc4ccc5ccc6c(c5c4c3)-c3ccccc3C6(C)C)n1)c1c2oc2ccccc21 ; CC1(C)c2ccccc2-c2c1ccc1ccc3ccc(-c4nc(-c5ccc6sc7ccccc7c6c5)nc(-c5c6c(cc7oc8ccccc8c57)-c5ccccc5C6(C)C)n4)cc3c21 ; CC1(C)c2ccccc2-c2ccc(-c3nc(-c4ccc(-c5cc6c(c7oc8ccccc8c57)-c5ccccc5C6(C)C)cc4)nc(-c4ccc5ccc6ccc7c(c6c5c4)-c4ccccc4C7(C)C)n3)cc21]

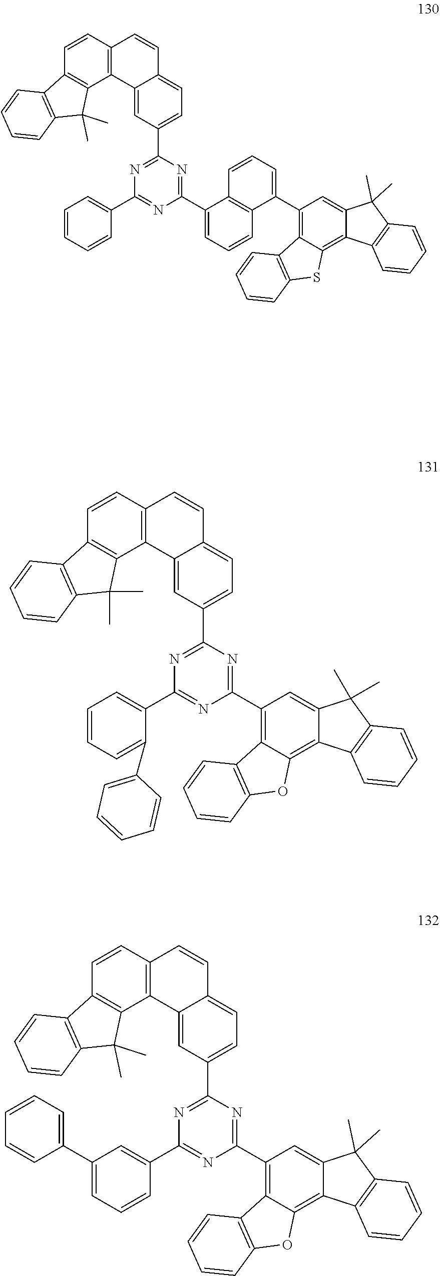 [SMILES: CC1(C)c2ccccc2-c2c1cc(-c1cccc3c(-c4nc(-c5ccccc5)nc(-c5ccc6ccc7ccc8c(c7c6c5)C(C)(C)c5ccccc5-8)n4)cccc13)c1c2sc2ccccc21 ; CC1(C)c2ccccc2-c2c1cc(-c1nc(-c3ccc4ccc5ccc6c(c5c4c3)C(C)(C)c3ccccc3-6)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5ccc6c(c5c4c3)C(C)(C)c3ccccc3-6)n1)c1c2oc2ccccc21]

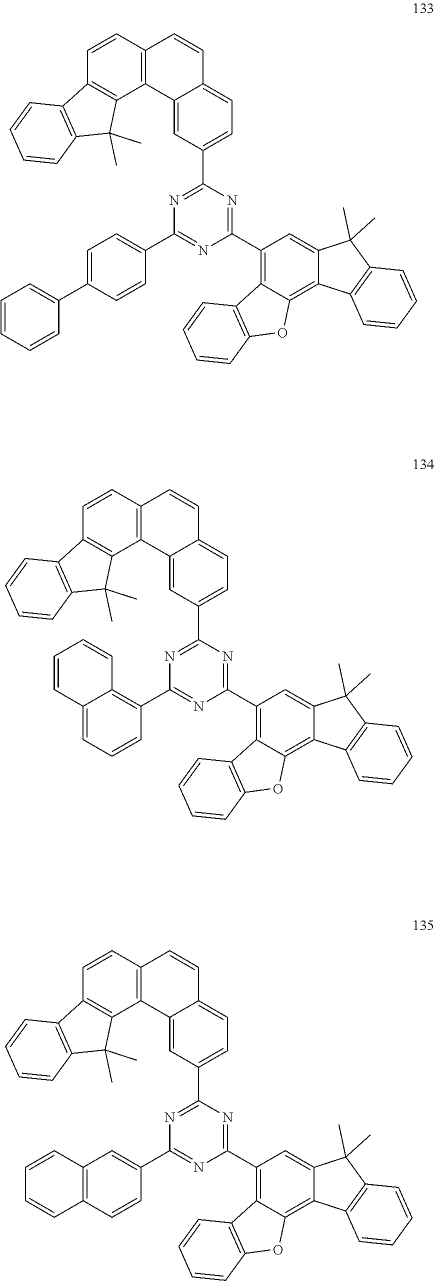 [SMILES: CC1(C)c2ccccc2-c2c1cc(-c1nc(-c3ccc(-c4ccccc4)cc3)nc(-c3ccc4ccc5ccc6c(c5c4c3)C(C)(C)c3ccccc3-6)n1)c1c2oc2ccccc21 ; CC1(C)c2ccccc2-c2c1cc(-c1nc(-c3ccc4ccc5ccc6c(c5c4c3)C(C)(C)c3ccccc3-6)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6c(c5c4c3)C(C)(C)c3ccccc3-6)n1)c1c2oc2ccccc21]

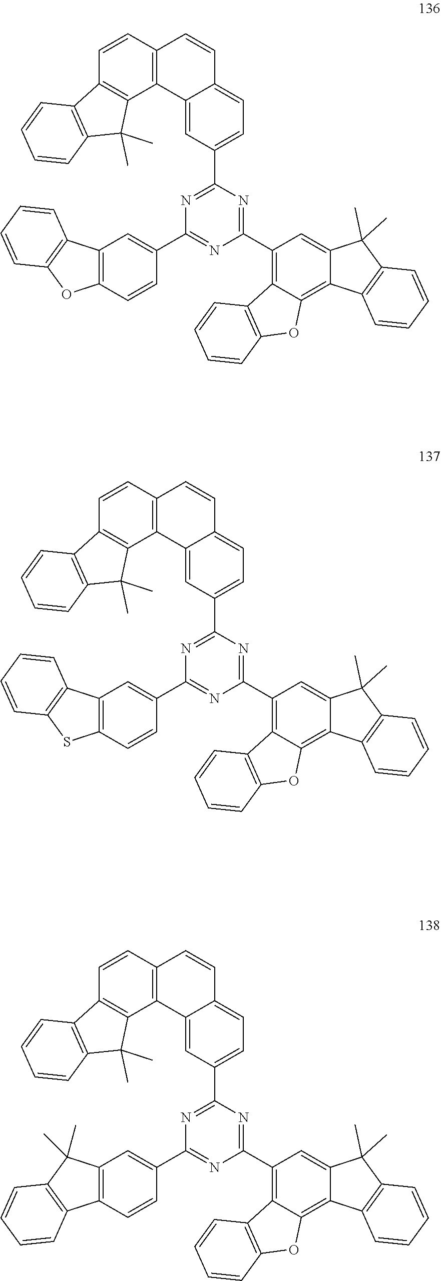 [SMILES: CC1(C)c2ccccc2-c2c1cc(-c1nc(-c3ccc4oc5ccccc5c4c3)nc(-c3ccc4ccc5ccc6c(c5c4c3)C(C)(C)c3ccccc3-6)n1)c1c2oc2ccccc21 ; CC1(C)c2ccccc2-c2c1cc(-c1nc(-c3ccc4sc5ccccc5c4c3)nc(-c3ccc4ccc5ccc6c(c5c4c3)C(C)(C)c3ccccc3-6)n1)c1c2oc2ccccc21 ; CC1(C)c2ccccc2-c2ccc(-c3nc(-c4ccc5ccc6ccc7c(c6c5c4)C(C)(C)c4ccccc4-7)nc(-c4cc5c(c6oc7ccccc7c46)-c4ccccc4C5(C)C)n3)cc21]

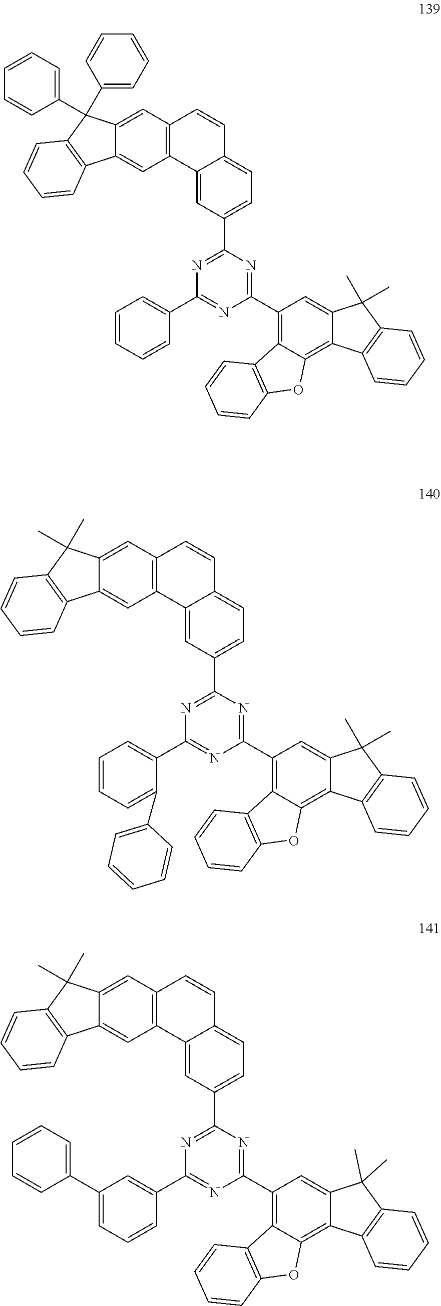 [SMILES: CC1(C)c2ccccc2-c2c1cc(-c1nc(-c3ccccc3)nc(-c3ccc4ccc5cc6c(cc5c4c3)-c3ccccc3C6(c3ccccc3)c3ccccc3)n1)c1c2oc2ccccc21 ; CC1(C)c2ccccc2-c2cc3c(ccc4ccc(-c5nc(-c6ccccc6-c6ccccc6)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6cccc(-c7ccccc7)c6)nc(-c6cc7c(c8oc9ccccc9c68)-c6ccccc6C7(C)C)n5)cc43)cc21]

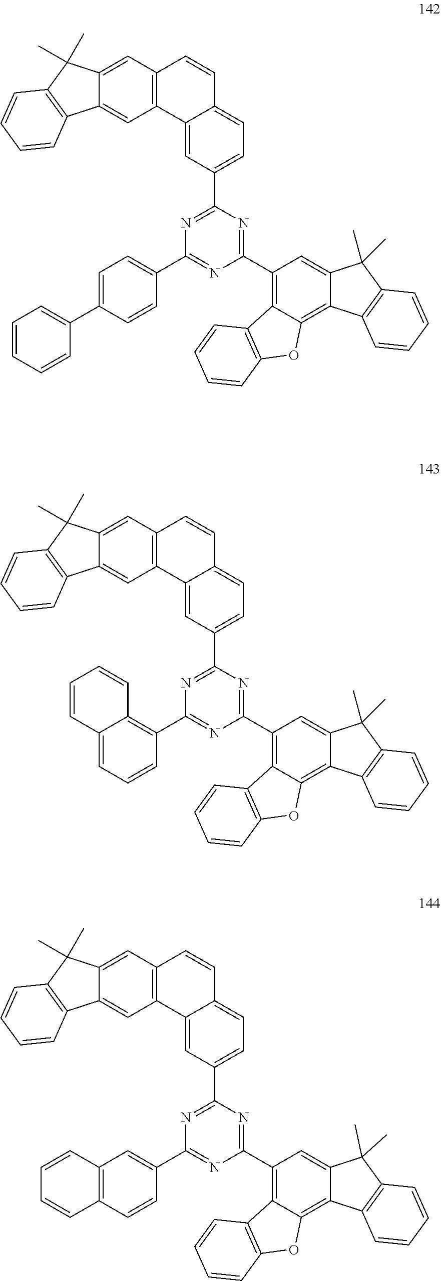 [SMILES: CC1(C)c2ccccc2-c2cc3c(ccc4ccc(-c5nc(-c6ccc(-c7ccccc7)cc6)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6cccc7ccccc67)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6ccc7ccccc7c6)nc(-c6cc7c(c8oc9ccccc9c68)-c6ccccc6C7(C)C)n5)cc43)cc21]

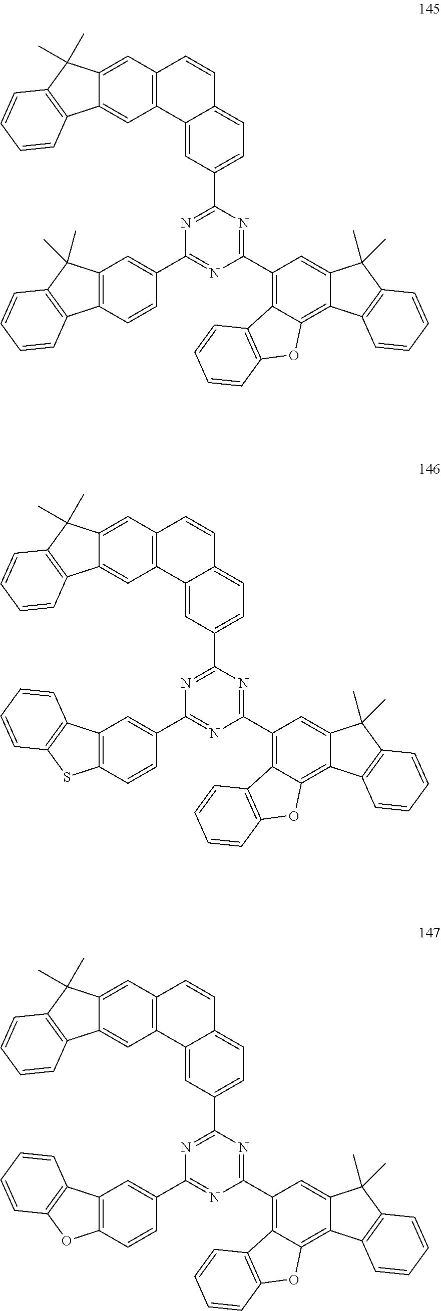 [SMILES: CC1(C)c2ccccc2-c2ccc(-c3nc(-c4ccc5ccc6cc7c(cc6c5c4)-c4ccccc4C7(C)C)nc(-c4cc5c(c6oc7ccccc7c46)-c4ccccc4C5(C)C)n3)cc21 ; CC1(C)c2ccccc2-c2cc3c(ccc4ccc(-c5nc(-c6ccc7sc8ccccc8c7c6)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6ccc7oc8ccccc8c7c6)nc(-c6cc7c(c8oc9ccccc9c68)-c6ccccc6C7(C)C)n5)cc43)cc21]

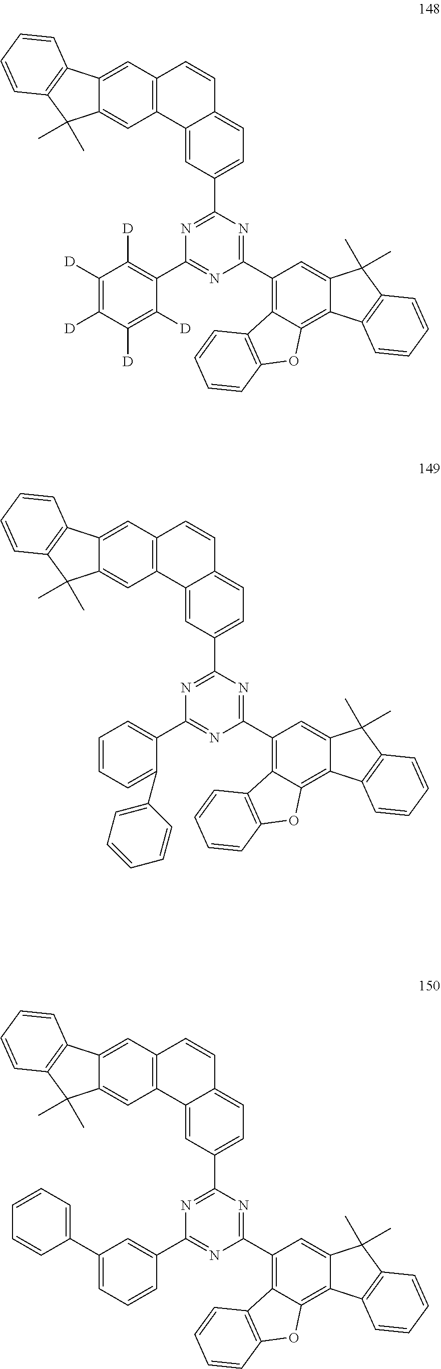 [SMILES: [2H]c1c([2H])c([2H])c(-c2nc(-c3ccc4ccc5cc6c(cc5c4c3)C(C)(C)c3ccccc3-6)nc(-c3cc4c(c5oc6ccccc6c35)-c3ccccc3C4(C)C)n2)c([2H])c1[2H] ; CC1(C)c2ccccc2-c2cc3ccc4ccc(-c5nc(-c6ccccc6-c6ccccc6)nc(-c6cc7c(c8oc9ccccc9c68)-c6ccccc6C7(C)C)n5)cc4c3cc21 ; CC1(C)c2ccccc2-c2cc3ccc4ccc(-c5nc(-c6cccc(-c7ccccc7)c6)nc(-c6cc7c(c8oc9ccccc9c68)-c6ccccc6C7(C)C)n5)cc4c3cc21]

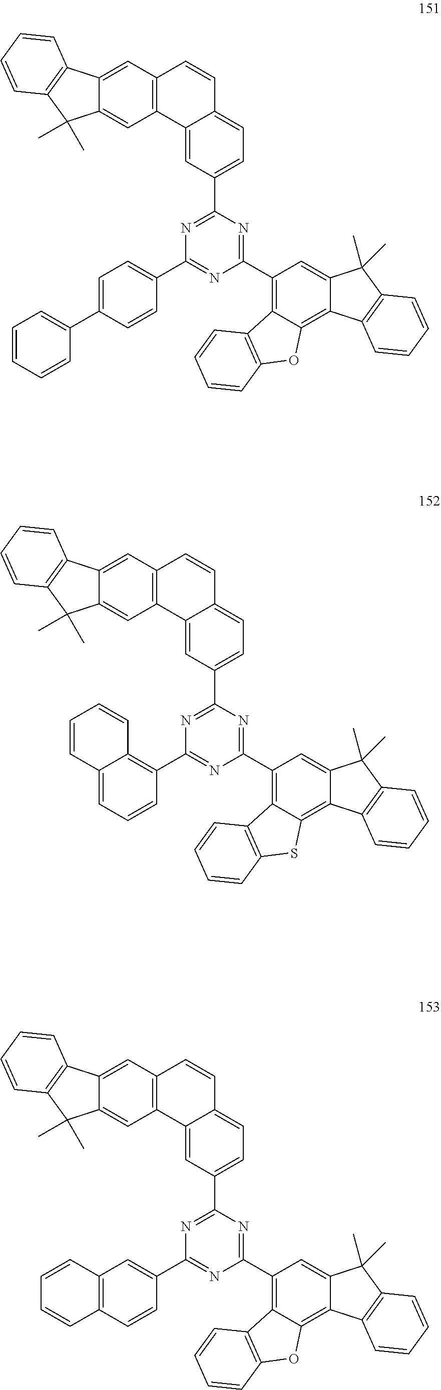 [SMILES: CC1(C)c2ccccc2-c2cc3ccc4ccc(-c5nc(-c6ccc(-c7ccccc7)cc6)nc(-c6cc7c(c8oc9ccccc9c68)-c6ccccc6C7(C)C)n5)cc4c3cc21 ; CC1(C)c2ccccc2-c2cc3ccc4ccc(-c5nc(-c6cccc7ccccc67)nc(-c6cc7c(c8sc9ccccc9c68)-c6ccccc6C7(C)C)n5)cc4c3cc21 ; CC1(C)c2ccccc2-c2cc3ccc4ccc(-c5nc(-c6ccc7ccccc7c6)nc(-c6cc7c(c8oc9ccccc9c68)-c6ccccc6C7(C)C)n5)cc4c3cc21]

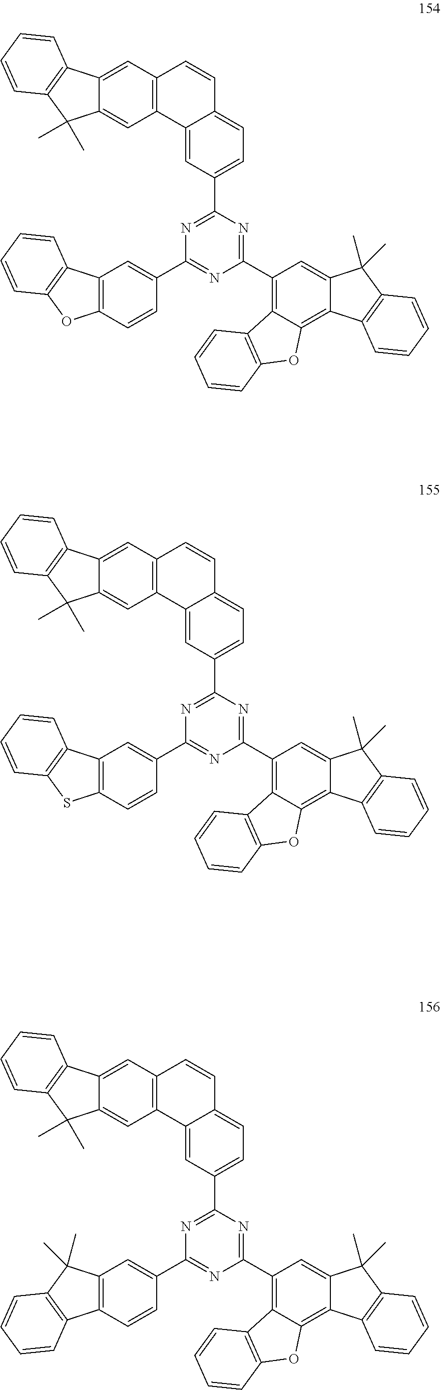 [SMILES: CC1(C)c2ccccc2-c2cc3ccc4ccc(-c5nc(-c6ccc7oc8ccccc8c7c6)nc(-c6cc7c(c8oc9ccccc9c68)-c6ccccc6C7(C)C)n5)cc4c3cc21 ; CC1(C)c2ccccc2-c2cc3ccc4ccc(-c5nc(-c6ccc7sc8ccccc8c7c6)nc(-c6cc7c(c8oc9ccccc9c68)-c6ccccc6C7(C)C)n5)cc4c3cc21 ; CC1(C)c2ccccc2-c2ccc(-c3nc(-c4ccc5ccc6cc7c(cc6c5c4)C(C)(C)c4ccccc4-7)nc(-c4cc5c(c6oc7ccccc7c46)-c4ccccc4C5(C)C)n3)cc21]

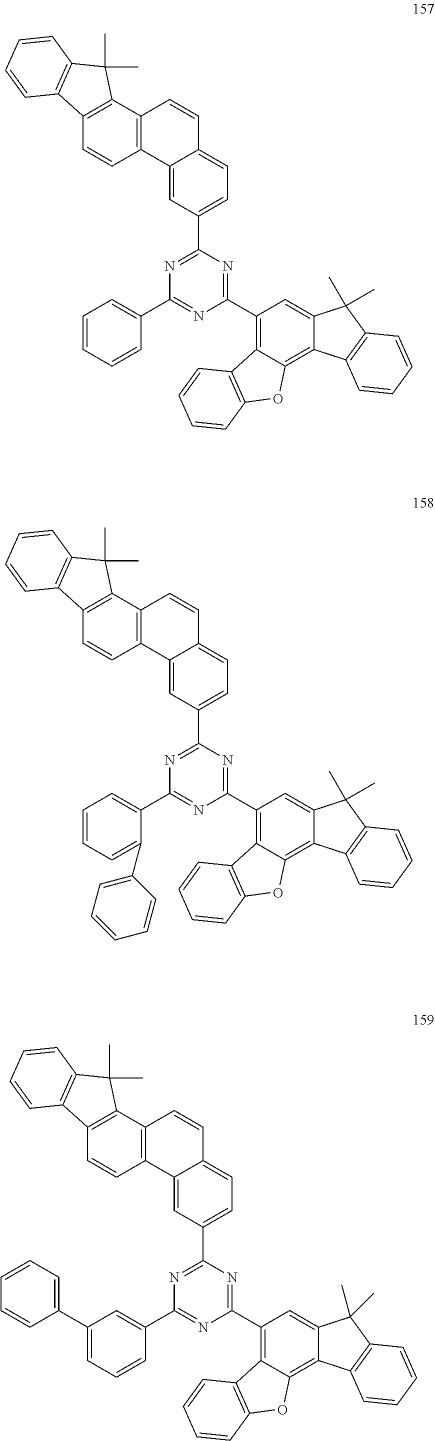 [SMILES: CC1(C)c2ccccc2-c2c1cc(-c1nc(-c3ccccc3)nc(-c3ccc4ccc5c6c(ccc5c4c3)-c3ccccc3C6(C)C)n1)c1c2oc2ccccc21 ; CC1(C)c2ccccc2-c2c1cc(-c1nc(-c3ccc4ccc5c6c(ccc5c4c3)-c3ccccc3C6(C)C)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5c6c(ccc5c4c3)-c3ccccc3C6(C)C)n1)c1c2oc2ccccc21]

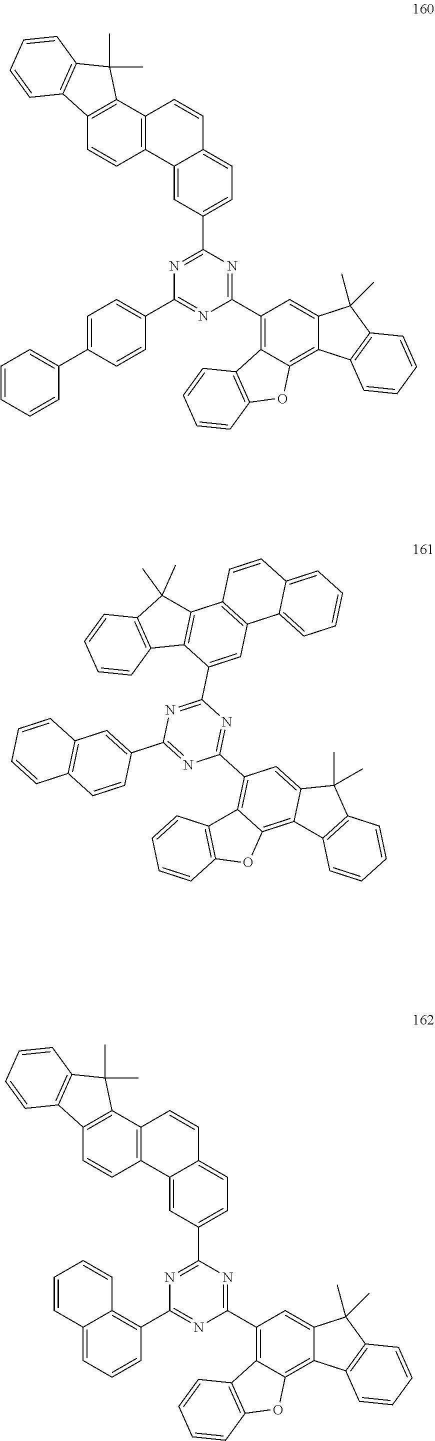 [SMILES: CC1(C)c2ccccc2-c2c1cc(-c1nc(-c3ccc(-c4ccccc4)cc3)nc(-c3ccc4ccc5c6c(ccc5c4c3)-c3ccccc3C6(C)C)n1)c1c2oc2ccccc21 ; CC1(C)c2ccccc2-c2c1cc(-c1nc(-c3ccc4ccccc4c3)nc(-c3cc4c(ccc5ccccc54)c4c3-c3ccccc3C4(C)C)n1)c1c2oc2ccccc21 ; CC1(C)c2ccccc2-c2c1cc(-c1nc(-c3ccc4ccc5c6c(ccc5c4c3)-c3ccccc3C6(C)C)nc(-c3cccc4ccccc34)n1)c1c2oc2ccccc21]

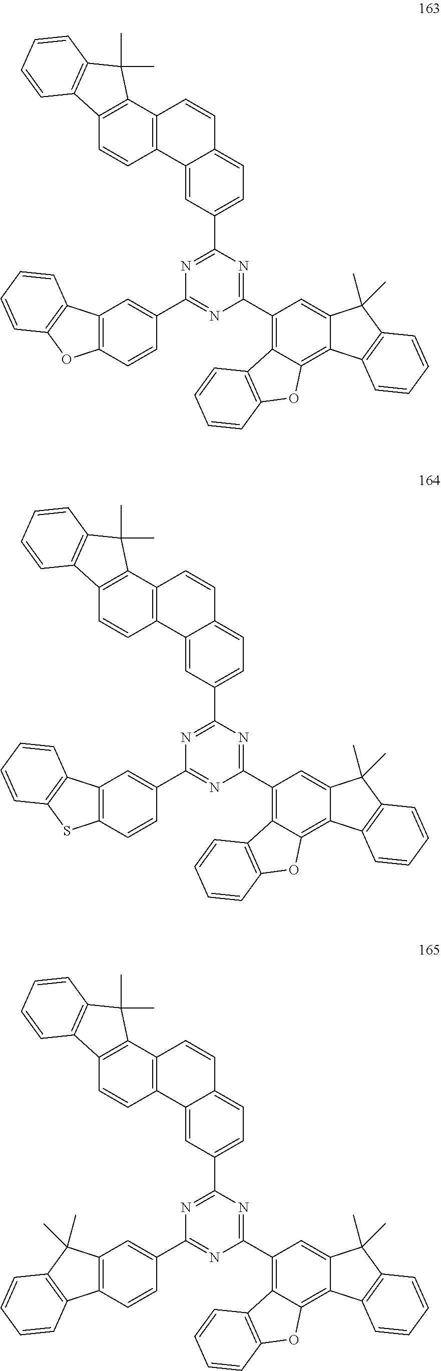 [SMILES: CC1(C)c2ccccc2-c2c1cc(-c1nc(-c3ccc4ccc5c6c(ccc5c4c3)-c3ccccc3C6(C)C)nc(-c3ccc4oc5ccccc5c4c3)n1)c1c2oc2ccccc21 ; CC1(C)c2ccccc2-c2c1cc(-c1nc(-c3ccc4ccc5c6c(ccc5c4c3)-c3ccccc3C6(C)C)nc(-c3ccc4sc5ccccc5c4c3)n1)c1c2oc2ccccc21 ; CC1(C)c2ccccc2-c2ccc(-c3nc(-c4ccc5ccc6c7c(ccc6c5c4)-c4ccccc4C7(C)C)nc(-c4cc5c(c6oc7ccccc7c46)-c4ccccc4C5(C)C)n3)cc21]

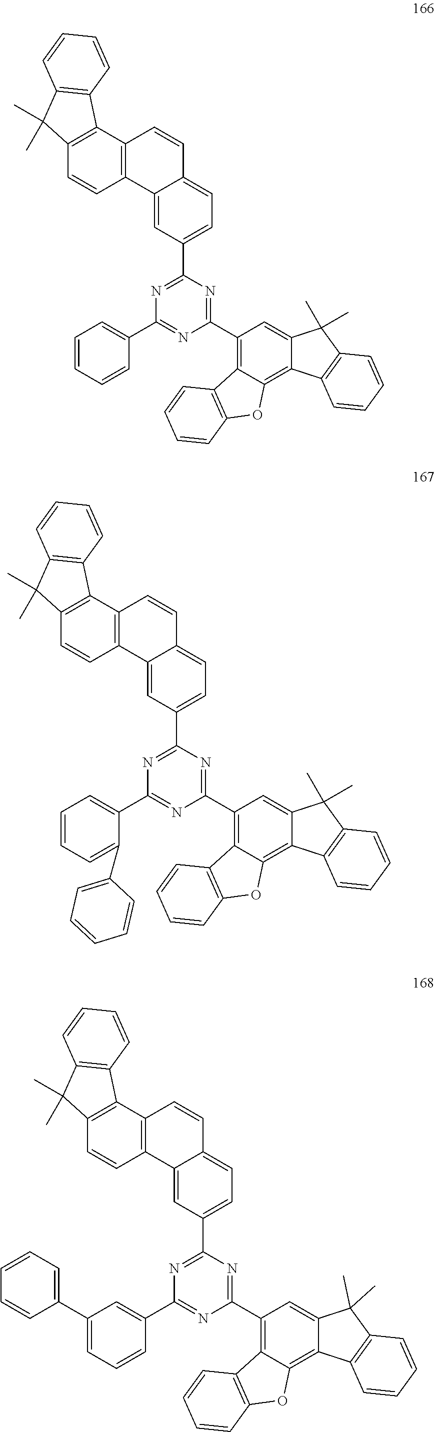 [SMILES: CC1(C)c2ccccc2-c2c1ccc1c2ccc2ccc(-c3nc(-c4ccccc4)nc(-c4cc5c(c6oc7ccccc7c46)-c4ccccc4C5(C)C)n3)cc21 ; CC1(C)c2ccccc2-c2c1ccc1c2ccc2ccc(-c3nc(-c4ccccc4-c4ccccc4)nc(-c4cc5c(c6oc7ccccc7c46)-c4ccccc4C5(C)C)n3)cc21 ; CC1(C)c2ccccc2-c2c1ccc1c2ccc2ccc(-c3nc(-c4cccc(-c5ccccc5)c4)nc(-c4cc5c(c6oc7ccccc7c46)-c4ccccc4C5(C)C)n3)cc21]

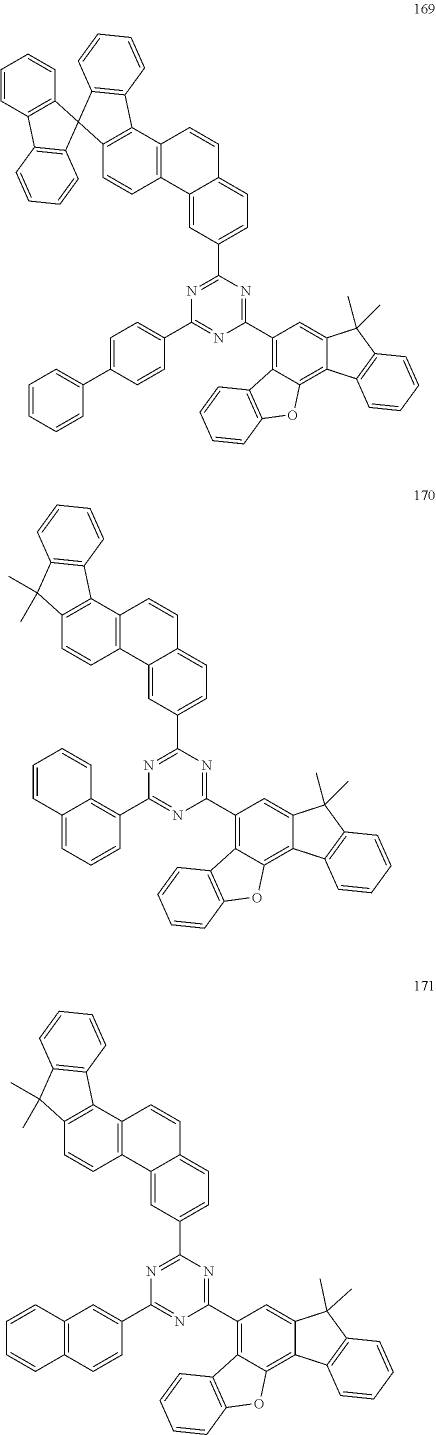

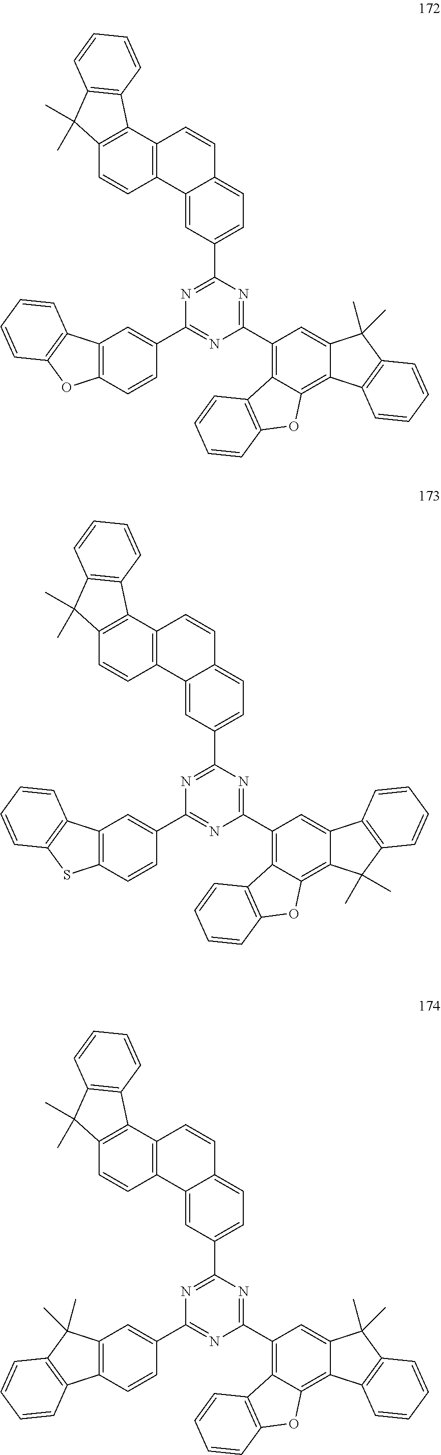 [SMILES: CC1(C)c2ccccc2-c2c1ccc1c2ccc2ccc(-c3nc(-c4ccc5oc6ccccc6c5c4)nc(-c4cc5c(c6oc7ccccc7c46)-c4ccccc4C5(C)C)n3)cc21 ; CC1(C)c2ccccc2-c2c1ccc1c2ccc2ccc(-c3nc(-c4ccc5sc6ccccc6c5c4)nc(-c4cc5c(c6oc7ccccc7c46)C(C)(C)c4ccccc4-5)n3)cc21 ; CC1(C)c2ccccc2-c2ccc(-c3nc(-c4ccc5ccc6c7c(ccc6c5c4)C(C)(C)c4ccccc4-7)nc(-c4cc5c(c6oc7ccccc7c46)-c4ccccc4C5(C)C)n3)cc21]

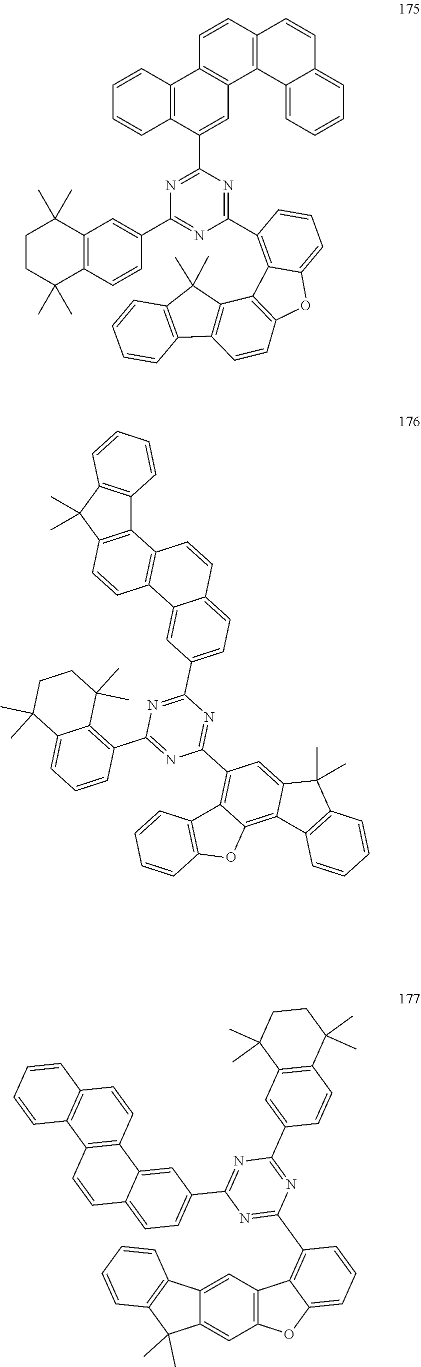 [SMILES: CC1(C)CCC(C)(C)c2cc(-c3nc(-c4cc5c(ccc6ccc7ccccc7c65)c5ccccc45)nc(-c4cccc5oc6ccc7c(c6c45)C(C)(C)c4ccccc4-7)n3)ccc21 ; CC1(C)CCC(C)(C)c2c(-c3nc(-c4ccc5ccc6c7c(ccc6c5c4)C(C)(C)c4ccccc4-7)nc(-c4cc5c(c6oc7ccccc7c46)-c4ccccc4C5(C)C)n3)cccc21 ; CC1(C)CCC(C)(C)c2cc(-c3nc(-c4ccc5ccc6c7ccccc7ccc6c5c4)nc(-c4cccc5oc6cc7c(cc6c45)-c4ccccc4C7(C)C)n3)ccc21]

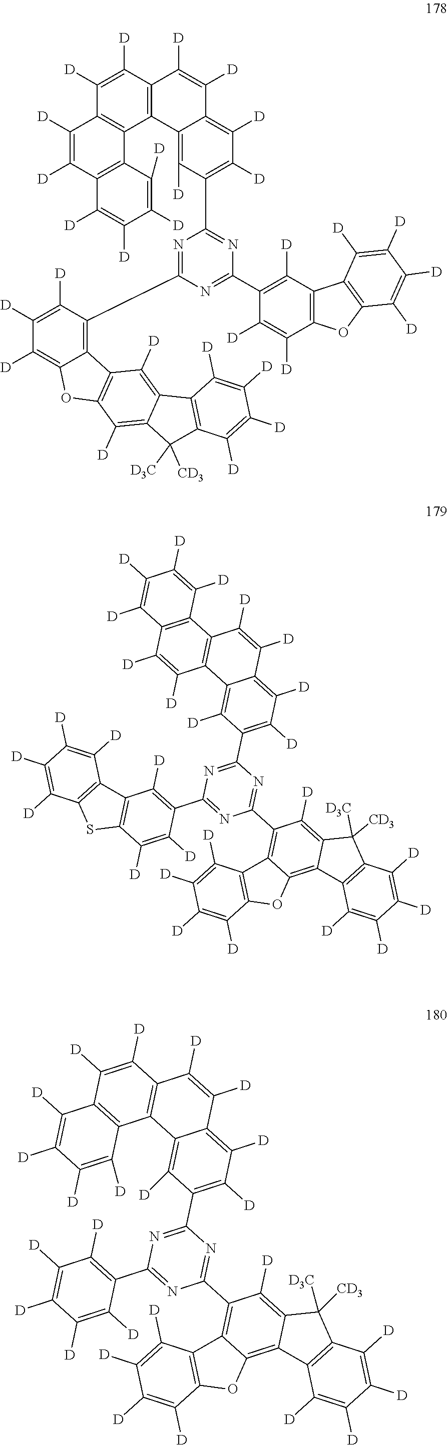

However, these compounds are not intended to limit the scope of Formula 1.

A further aspect of the present invention is directed to an organic light emitting device including a first electrode, a second electrode, and one or more organic layers interposed between the first and second electrodes wherein one of the organic layers, preferably a light emitting layer includes the compound represented by Formula 1 as a host.

The light emitting layer may further include a dopant. In this case, the content of the dopant in the light emitting layer is typically in the range of about 0.01 to about 20 parts by weight, based on about 100 parts by weight of the host but is not limited to this range.

The light emitting layer may further include one or more other hosts and one or more other dopant materials. The hosts and the dopant materials may be mixed or stacked in the light emitting layer.

The organic layers of the organic light emitting device according to the present invention may form a monolayer structure. Alternatively, the organic layers may be stacked together to form a multilayer structure. For example, the organic layers may have a structure including a hole injecting layer, a hole transport layer, a hole blocking layer, a light emitting layer, an electron blocking layer, an electron transport layer, and an electron injecting layer but are not limited to this structure. The number of the organic layers is not limited and may be increased or decreased. Preferred structures of the organic layers of the organic light emitting device according to the present invention will be explained in more detail in the Examples section that follows.

A more detailed description will be given concerning exemplary embodiments of the organic light emitting device according to the present invention.

The organic light emitting device of the present invention includes an anode, a hole transport layer, a light emitting layer, an electron transport layer, and a cathode. The organic light emitting device of the present invention may optionally further include a hole injecting layer between the anode and the hole transport layer and an electron injecting layer between the electron transport layer and the cathode. If necessary, the organic light emitting device of the present invention may further include one or two intermediate layers such as a hole blocking layer or an electron blocking layer. The organic light emitting device of the present invention may further include one or more organic layers such as a capping layer that have various functions depending on the desired characteristics of the device.

A specific structure of the organic light emitting device according to one embodiment of the present invention, a method for fabricating the device, and materials for the organic layers are as follows.

First, an anode material is coated on a substrate to form an anode. The substrate may be any of those used in general organic light emitting devices. The substrate is preferably an organic substrate or a transparent plastic substrate that is excellent in transparency, surface smoothness, ease of handling, and waterproofness. A highly transparent and conductive metal oxide such as indium tin oxide (ITO), indium zinc oxide (IZO), tin oxide (SnO₂) or zinc oxide (ZnO) is used as the anode material.

A hole injecting material is coated on the anode by vacuum thermal evaporation or spin coating to form a hole injecting layer. Then, a hole transport material is coated on the hole injecting layer by vacuum thermal evaporation or spin coating to form a hole transport layer.

The hole injecting material is not specially limited so long as it is usually used in the art. Specific examples of such materials include 4,4′,4″-tris(2-naphthylphenyl-phenylamino)triphenylamine (2-TNATA), N,N′-di(1-naphthyl)-N,N′-diphenylbenzidine (NPD), N,N′-diphenyl-N,N′-bis(3-methylphenyl)-1,1′-biphenyl-4,4′-diamine (TPD), and N,N′-diphenyl-N,N′-bis(4-(phenyl-m-tolylamino)phenyl)biphenyl-4,4′-diamine (DNTPD).

The hole transport material is not specially limited so long as it is commonly used in the art. Examples of such materials include N,N′-bis(3-methylphenyl)-N,N′-diphenyl-(1,1-biphenyl)-4,4′-diamine (TPD) and N,N′-di(naphthalen-1-yl)-N,N′-diphenylbenzidine (α-NPD).

Subsequently, a hole auxiliary layer and a light emitting layer are sequentially laminated on the hole transport layer. A hole blocking layer may be optionally formed on the light emitting layer by vacuum thermal evaporation or spin coating. The hole blocking layer is formed as a thin film and blocks holes from entering a cathode through the organic light emitting layer. This role of the hole blocking layer prevents the lifetime and efficiency of the device from deteriorating. A material having a very low highest occupied molecular orbital (HOMO) energy level is used for the hole blocking layer. The hole blocking material is not particularly limited so long as it can transport electrons and has a higher ionization potential than the light emitting compound. Representative examples of suitable hole blocking materials include BAlq, BCP, and TPBI.

Examples of materials for the hole blocking layer include, but are not limited to, BAlq, BCP, Bphen, TPBI, TAZ, BeBq₂, OXD-7, and Liq.

An electron transport layer is deposited on the hole blocking layer by vacuum thermal evaporation or spin coating, and an electron injecting layer is formed thereon. A cathode metal is deposited on the electron injecting layer by vacuum thermal evaporation to form a cathode, completing the fabrication of the organic light emitting device.

For example, lithium (Li), magnesium (Mg), aluminum (Al), aluminum-lithium (Al—Li), calcium (Ca), magnesium-indium (Mg—In) or magnesium-silver (Mg—Ag) may be used as the metal for the formation of the cathode. The organic light emitting device may be of top emission type. In this case, a transmissive material such as ITO or IZO may be used to form the cathode.

A material for the electron transport layer functions to stably transport electrons injected from the cathode. The electron transport material may be any of those known in the art and examples thereof include, but are not limited to, quinoline derivatives, particularly tris(8-quinolinolato)aluminum (Alq3), TAZ, Balq, beryllium bis(benzoquinolin-10-olate) (Bebq2), and oxadiazole derivatives such as PBD, BMD, and BND.

Each of the organic layers can be formed by a monomolecular deposition or solution process. According to the monomolecular deposition process, the material for each layer is evaporated into a thin film under heat and vacuum or reduced pressure. According to the solution process, the material for each layer is mixed with a suitable solvent and the mixture is then formed into a thin film by a suitable method such as ink-jet printing, roll-to-roll coating, screen printing, spray coating, dip coating or spin coating.

The organic light emitting device of the present invention can be used in a display or lighting system selected from flat panel displays, flexible displays, monochromatic flat panel lighting systems, white flat panel lighting systems, flexible monochromatic lighting systems, flexible white lighting systems, displays for automotive applications, displays for virtual reality, and displays for augmented reality.

The present invention will be more specifically explained with reference to the following synthesis examples and fabrication examples. However, these examples are provided to assist in understanding the invention and are not intended to limit the scope of the present invention.

### Synthesis Example 1: Synthesis of Compound 55

### Synthesis Example 1-1: Synthesis of A-1

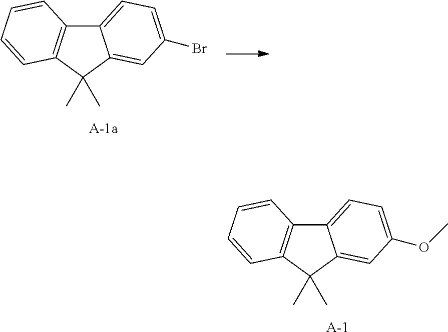

50 g of A-1a, 59.3 g of an aqueous solution of sodium methoxide, 10.4 g of copper iodide, and 200 mL of methanol were placed in a nitrogen-purged reactor and heated to 70° C. After 12 h, the temperature was allowed to drop to room temperature and ethyl acetate and water were added for layer separation. The organic layer was concentrated under reduced pressure and purified by silica gel column chromatography to afford A-1 (33.2 g, 81%).

### Synthesis Example 1-2: Synthesis of A-2

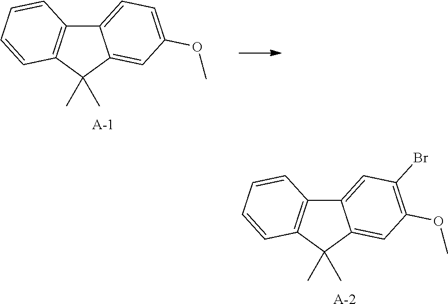

30 g of A-1, 23.8 g of N-bromosuccinimide, and 600 mL of dimethylformamide were placed in a nitrogen-purged reactor and heated to 50° C. After 12 h, the temperature was allowed to drop to room temperature and ethyl acetate and water were added for layer separation. The organic layer was concentrated under reduced pressure and purified by silica gel column chromatography to afford A-2 (28 g, 70%).

### Synthesis Example 1-3: Synthesis of A-3

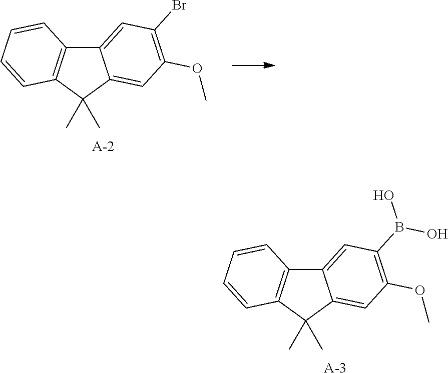

17.6 g of A-2 and 200 mL of tetrahydrofuran were placed in a nitrogen-purged reactor, cooled to −78° C., and dropwise added with 36.3 mL of 1.6 M butyllithium. 1 h later, 7 mL of trimethyl borate was added dropwise. After 2 h, the mixture was heated to 0° C. and acidified by addition of 1 M hydrochloric acid. The temperature was allowed to rise to room temperature and ethyl acetate and water were added for layer separation. The organic layer was concentrated under reduced pressure and recrystallized to afford A-3 (13 g, 82%).

### Synthesis Example 1-4: Synthesis of A-4

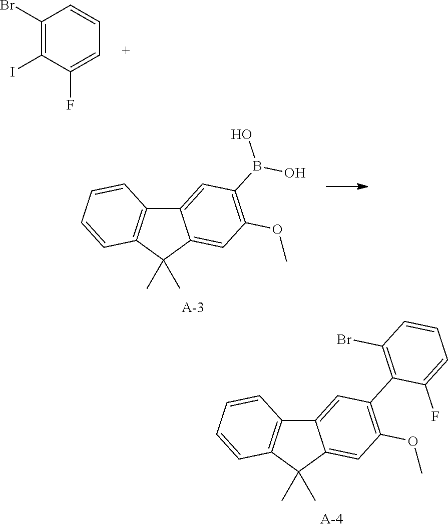

21 g of A-3, 18 g of A-4a, 1.8 g of tetrakis(triphenylphosphine)palladium(0), 21.7 g of potassium carbonate, 105 mL of toluene, 105 mL of 1,4-dioxane, and 42 mL of water were placed in a nitrogen-purged reactor and heated to 110° C. After 12 h, the temperature was allowed to drop to room temperature and ethyl acetate and water were added for layer separation. The organic layer was concentrated under reduced pressure and purified by silica gel column chromatography to afford A-4 (20.7 g, 67%).

### Synthesis Example 1-5: Synthesis of A-5

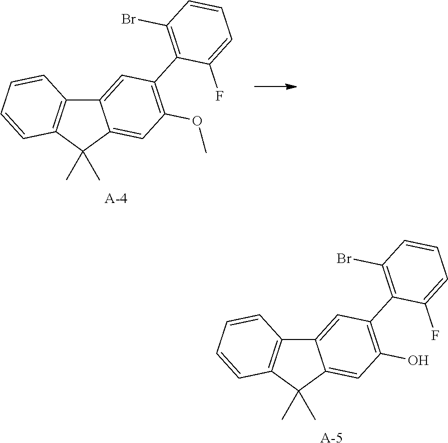

30 g of A-4 and 300 mL of dichloromethane were placed in a nitrogen-purged reactor and cooled to 0° C. To the mixture was added dropwise a solution of 56.7 g of boron tribromide in 150 mL of dichloromethane. The temperature was allowed to rise to room temperature. After 6 h, water was added for layer separation. The organic layer was concentrated under reduced pressure and purified by silica gel column chromatography to afford A-5 (21.3 g, 74%).

### Synthesis Example 1-6: Synthesis of A-6

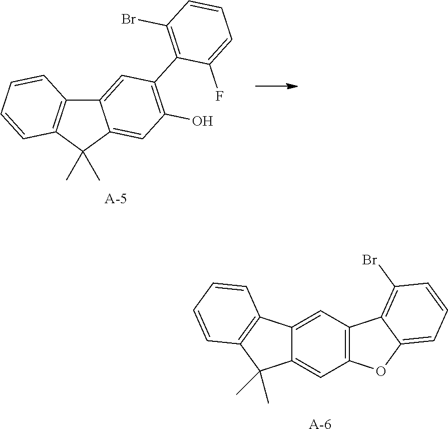

20 g of A-5, 11 g of potassium carbonate, and 200 mL of 1-methyl-2-pyrrolidinone were placed in a nitrogen-purged reactor and heated to 150° C. After 12 h, the temperature was allowed to drop to room temperature and ethyl acetate and water were added for layer separation. The organic layer was concentrated under reduced pressure and purified by silica gel column chromatography to afford A-6 (13.5 g, 71%).

### Synthesis Example 1-7: Synthesis of A-7

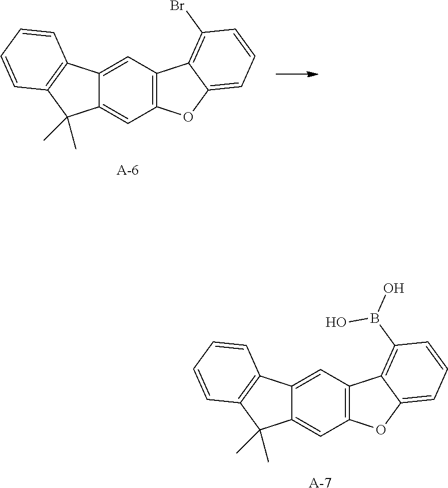 [SMILES: CC1(C)c2ccccc2-c2cc3c(cc21)oc1cccc(Br)c13 ; "A-6" ; CC1(C)c2ccccc2-c2cc3c(cc21)oc1cccc(B(O)O)c13 ; "A-7"]

A-7 (yield 75.3%) was synthesized in the same manner as in Synthesis Example 1-3, except that A-6 was used instead of A-2.

### Synthesis Example 1-8: Synthesis of A-8

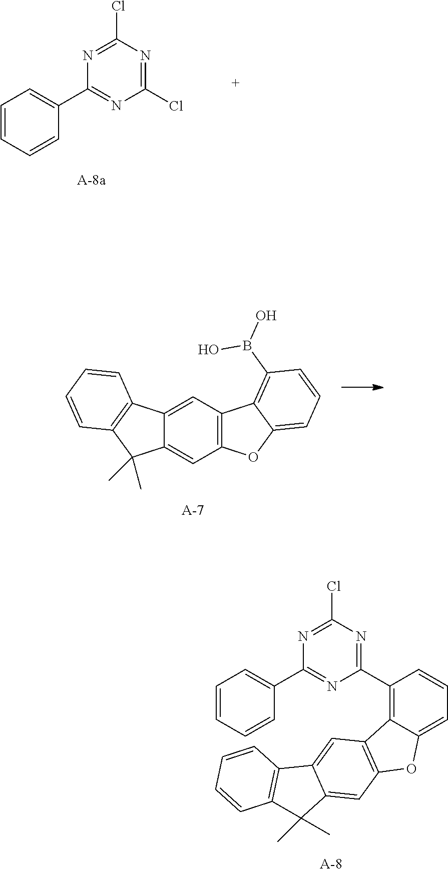

14.1 g of A-8a, 20 g of A-7, 0.86 g of bis(triphenylphosphine)palladium(II) dichloride, 60.9 mL of a 2 M aqueous solution of potassium carbonate, and 200 mL of toluene were placed in a nitrogen-purged reactor and heated to 110° C. After 6 h, the temperature was allowed to drop to room temperature and ethyl acetate and water were added for layer separation. The organic layer was concentrated under reduced pressure, added with toluene, dissolved by heating, filtered through silica gel, concentrated under reduced pressure, and recrystallized to afford A-8 (20 g, 69.2%).

### Synthesis Example 1-9: Synthesis of A-9

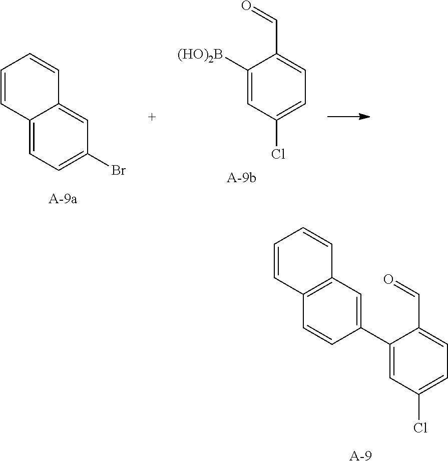 [SMILES: Brc1ccc2ccccc2c1 ; O=Cc1ccc(Cl)cc1B(O)O ; O=Cc1ccc(Cl)cc1-c1ccc2ccccc2c1 ; "A-9"]

84.4 g of A-9a, 82.7 g of A-9b, 9.42 g of tetrakis(triphenylphosphine)palladium(0), 140.8 g of potassium carbonate, 338 mL of toluene, 253 mL of ethanol, and 253 mL of water were placed in a nitrogen-purged reactor. After 6 h, ethyl acetate and water were added for layer separation. The organic layer was concentrated under reduced pressure and purified by silica gel column chromatography to afford A-9 (107.4 g, 98.8%).

### Synthesis Example 1-10: Synthesis of A-10

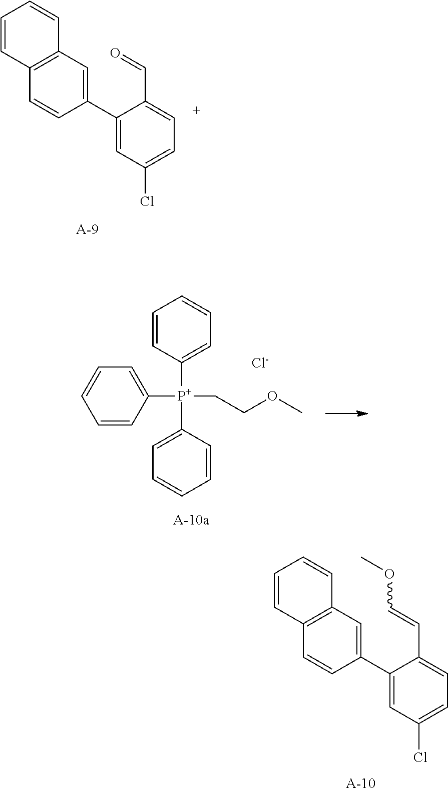 [SMILES: O=Cc1ccc(Cl)cc1-c1ccc2ccccc2c1 ; "A-9" ; COCC[P+](c1ccccc1)(c1ccccc1)c1ccccc1 ; [Cl-] ; COC=Cc1ccc(Cl)cc1-c1ccc2ccccc2c1 ; "A-10"]

43.2 g of potassium tert-butoxide and 1710 mL of tetrahydrofuran were placed in a nitrogen-purged reactor and cooled to −20° C. After 30 min, 131.9 g of A-10a was added. 30 min later, to the mixture were added dropwise a solution of 85.5 g of A-9 and 1710 mL of tetrahydrofuran. After 16 h, the temperature was allowed to rise to room temperature and ethyl acetate and water were added for layer separation. The organic layer was concentrated under reduced pressure and purified by silica gel column chromatography to afford A-10 (90 g, 95.2%).

### Synthesis Example 1-11: Synthesis of A-11

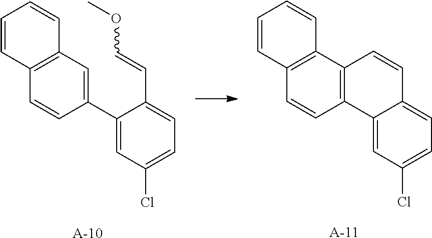 [SMILES: COC=Cc1ccc(Cl)cc1-c1ccc2ccccc2c1 ; Clc1ccc2ccc3c4ccccc4ccc3c2c1 ; "A-11" ; "A-10"]

90 g of A-10 and 900 mL of methylene chloride were placed in a nitrogen-purged reactor and 6.4 mL of methanesulfonic acid was added thereto at room temperature. After 2 h, the reaction solution was poured into methanol and filtered. The resulting solid was added with toluene, dissolved by heating, filtered through silica gel, concentrated under reduced pressure, and recrystallized to afford A-11 (53.7 g, 66.9%).

### Synthesis Example 1-12: Synthesis of A-12

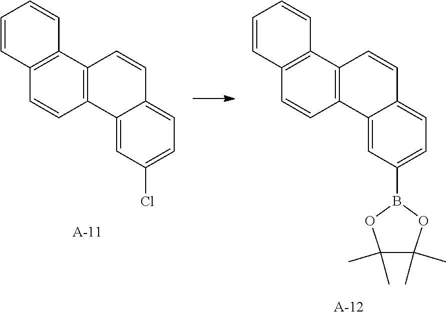

53.7 g of A-11, 77.9 g of bis(pinacolato)diboron, 100.3 g of potassium acetate, and 537 mL of 1,4-dioxane were placed in a nitrogen-purged reactor and heated to 50° C. To the mixture was added 29.9 g of 1,1′-bis(diphenylphosphino)ferrocene dichloropalladium(II). The temperature was raised to 100° C. After 6 h, 34 g of bis(pinacolato)diboron, 50 g of potassium acetate, and 15 g of 1,1′-bis(diphenylphosphino)ferrocene dichloropalladium(II) were further added. 16 h later, the resulting mixture was cooled to room temperature and ethyl acetate and water were added for layer separation. The organic layer was concentrated under reduced pressure, added with toluene, dissolved by heating, filtered through silica gel, concentrated under reduced pressure, and recrystallized to afford A-12 (53.4 g, 73.8%).

### Synthesis Example 1-13: Synthesis of Compound 55

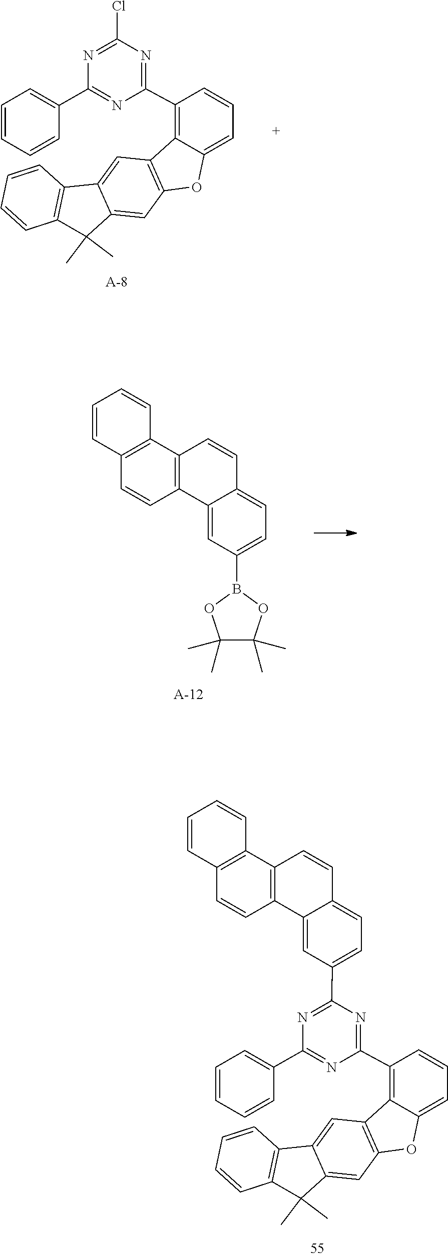

Compound 55 (yield 53.7%) was synthesized in the same manner as in Synthesis Example 1-9, except that A-8 and A-12 were used instead of A-9a and A-9b, respectively.

MS (MALDI-TOF): m/z 665.25 [M⁺]

### Synthesis Example 2: Synthesis of Compound 58

### Synthesis Example 2-1: Synthesis of Compound 58

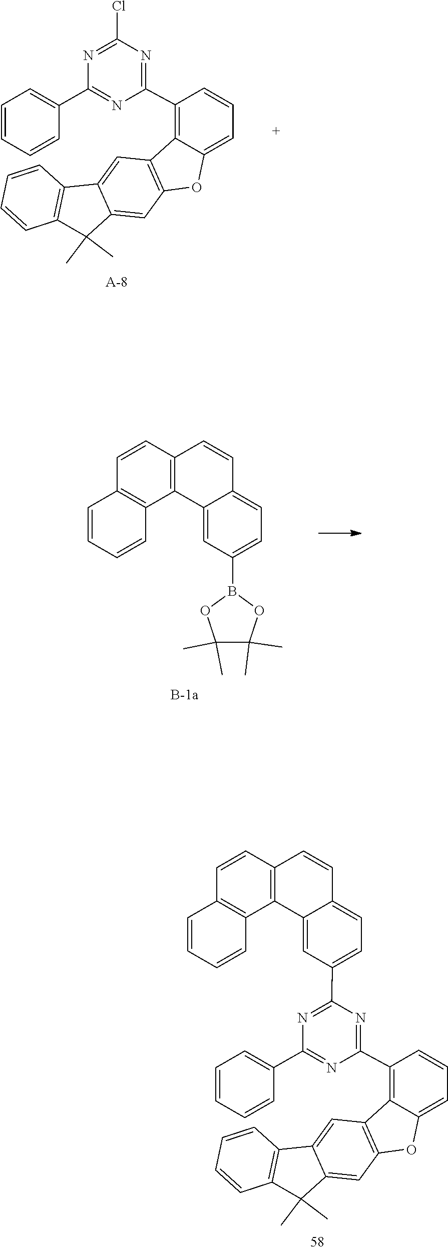

B-1a was synthesized according to the synthetic procedure described in Synthesis Examples 1-9 to 1-12, except that 1-bromonaphthalene was used instead of A-9a in Synthesis Example 1-9. Then, Compound 58 (yield 50.1%) was synthesized in the same manner as in Synthesis Example 1-13, except that B-1a was used instead of A-12.

MS (MALDI-TOF): m/z 665.25 [M⁺]

### Synthesis Example 3: Synthesis of Compound 61

### Synthesis Example 3-1: Synthesis of Compound 61

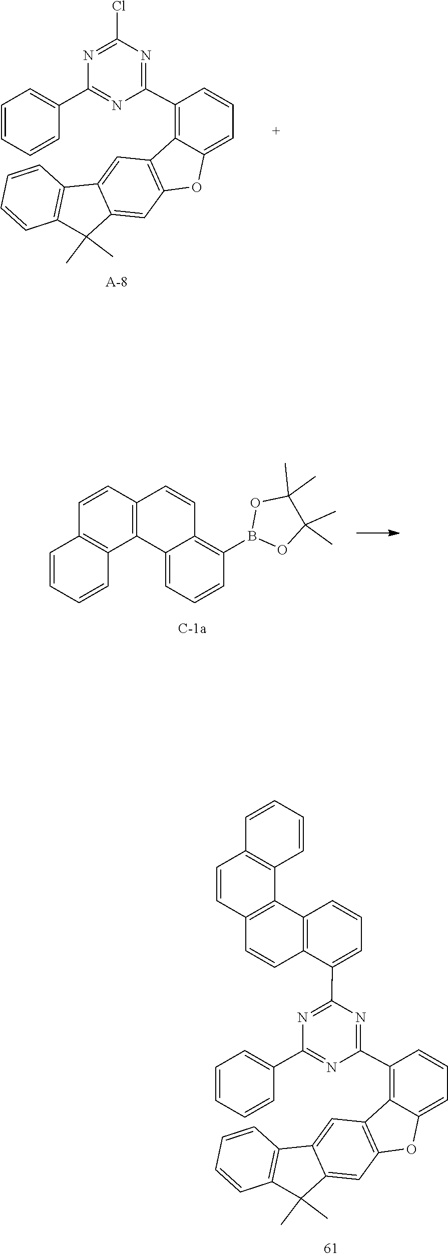

C-1a was synthesized according to the synthetic procedure described in Synthesis Examples 1-9 to 1-12, except that 1-bromonaphthalene and 3-chloro-2-formylphenylboronic acid were used instead of A-9a and A-9b in Synthesis Example 1-9, respectively. Then, Compound 61 (yield 56.3%) was synthesized in the same manner as in Synthesis Example 1-13, except that C-1a was used instead of A-12.

MS (MALDI-TOF): m/z 665.25 [M⁺]

### Synthesis Example 4: Synthesis of Compound 67

### Synthesis Example 4-1: Synthesis of Compound 67

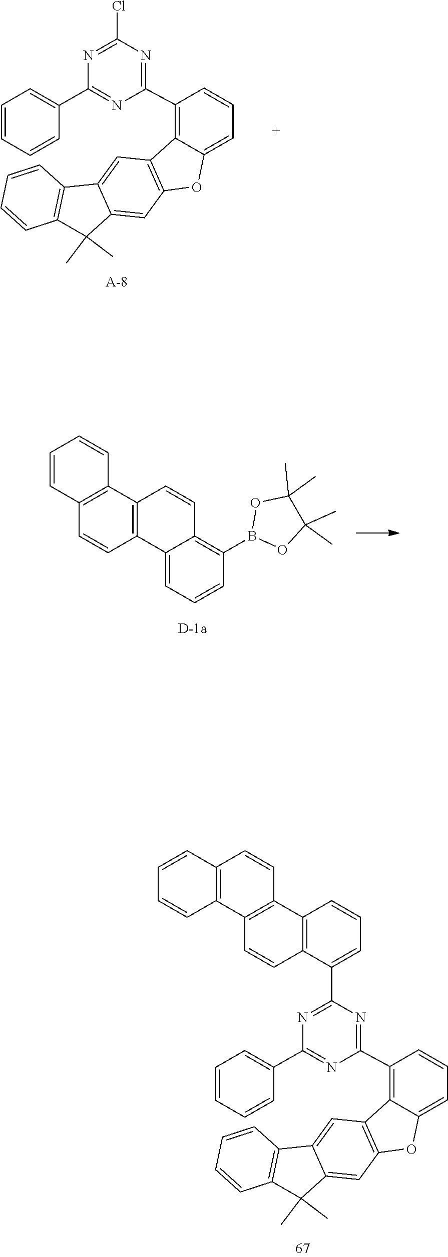

D-1a was synthesized according to the synthetic procedure described in Synthesis Examples 1-9 to 1-12, except that 3-chloro-2-formylphenylboronic acid was used instead of A-9b in Synthesis Example 1-9. Then, Compound 67 (yield 60%) was synthesized in the same manner as in Synthesis Example 1-13, except that D-1a was used instead of A-12.

MS (MALDI-TOF): m/z 665.25 [M⁺]

### Synthesis Example 5: Synthesis of Compound 68

### Synthesis Example 5-1: Synthesis of Compound 68

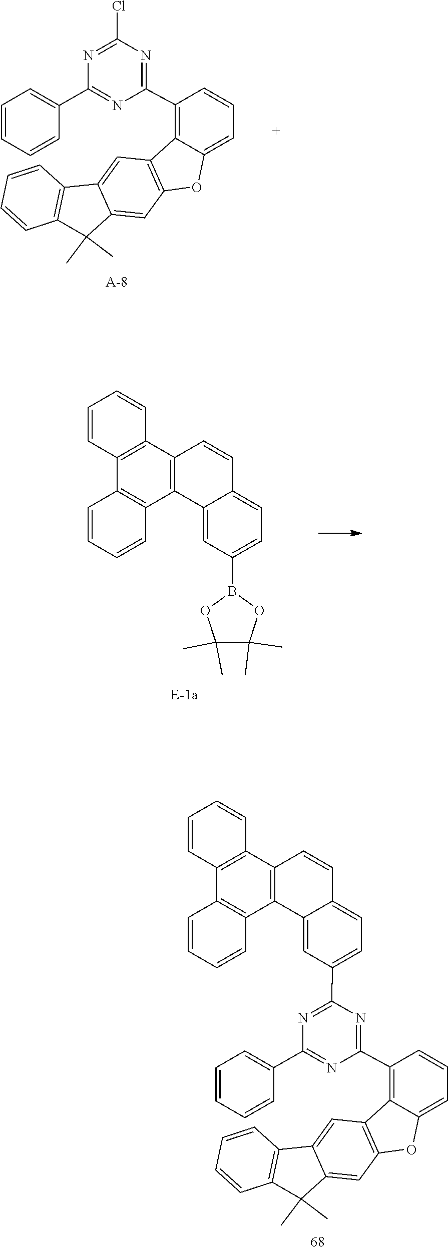

E-1a was synthesized according to the synthetic procedure described in Synthesis Examples 1-9 to 1-12, except that 9-bromophenanthrene was used instead of A-9a in Synthesis Example 1-9. Then, Compound 68 (yield 53.4%) was synthesized in the same manner as in Synthesis Example 1-13, except that E-1a was used instead of A-12.

MS (MALDI-TOF): m/z 715.26 [M⁺]

### Synthesis Example 6: Synthesis of Compound 79

### Synthesis Example 6-1: Synthesis of Compound 79

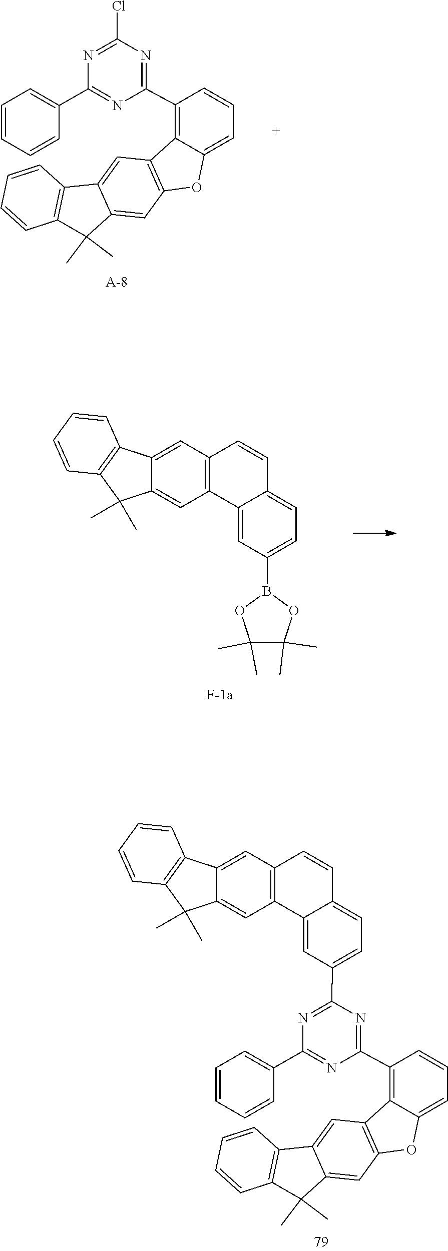

F-1a was synthesized according to the synthetic procedure described in Synthesis Examples 1-9 to 1-12, except that 2-bromo-9,9-dimethylfluorene was used instead of A-9a in Synthesis Example 1-9. Then, Compound 79 (yield 55.7%) was synthesized in the same manner as in Synthesis Example 1-13, except that F-1a was used instead of A-12.

MS (MALDI-TOF): m/z 731.29 [M⁺]

### Synthesis Example 7: Synthesis of Compound 80

### Synthesis Example 7-1: Synthesis of Compound 80

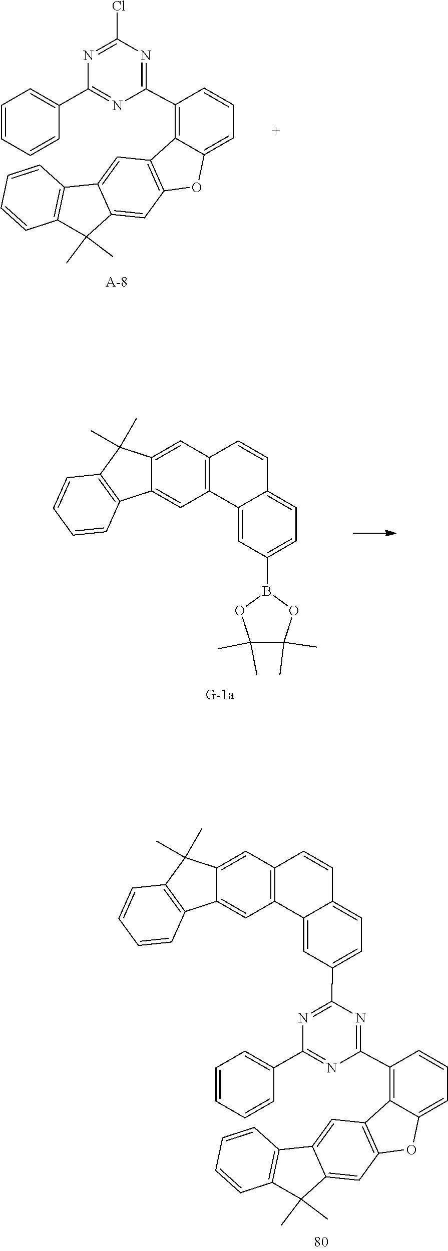

G-1a was synthesized according to the synthetic procedure described in Synthesis Examples 1-9 to 1-12, except that 3-bromo-9,9-dimethylfluorene was used instead of A-9a in Synthesis Example 1-9. Then, Compound 80 (yield 54.5%) was synthesized in the same manner as in Synthesis Example 1-13, except that G-1a was used instead of A-12.

MS (MALDI-TOF): m/z 731.29 [M⁺]

### Synthesis Example 8: Synthesis of Compound 81

### Synthesis Example 8-1: Synthesis of Compound 81

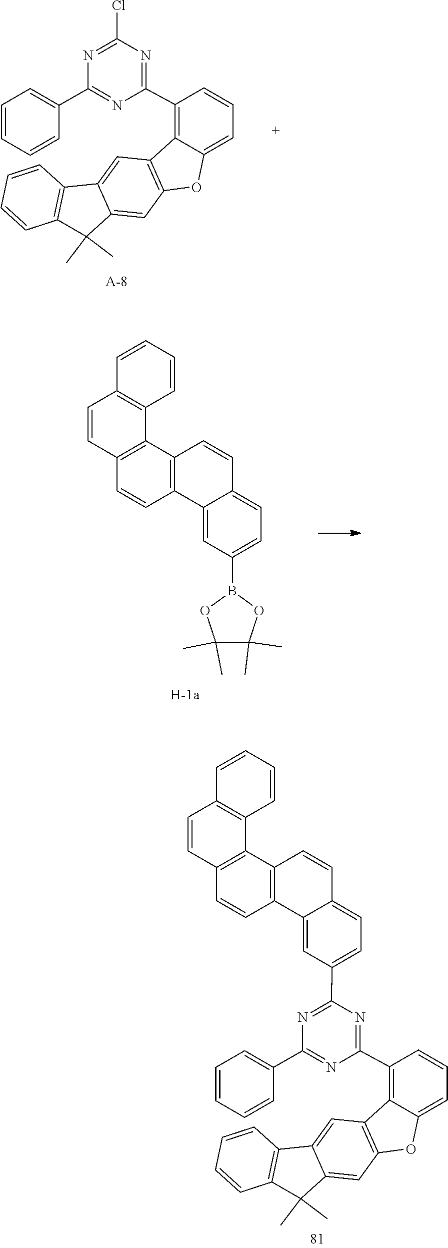

H-1a was synthesized according to the synthetic procedure described in Synthesis Examples 1-9 to 1-12, except that 3-bromophenanthrene was used instead of A-9a in Synthesis Example 1-9. Then, Compound 81 (yield 59.3%) was synthesized in the same manner as in Synthesis Example 1-13, except that H-1a was used instead of A-12.

MS (MALDI-TOF): m/z 715.26 [M⁺]

### Synthesis Example 9: Synthesis of Compound 88

### Synthesis Example 9-1: Synthesis of Compound 88

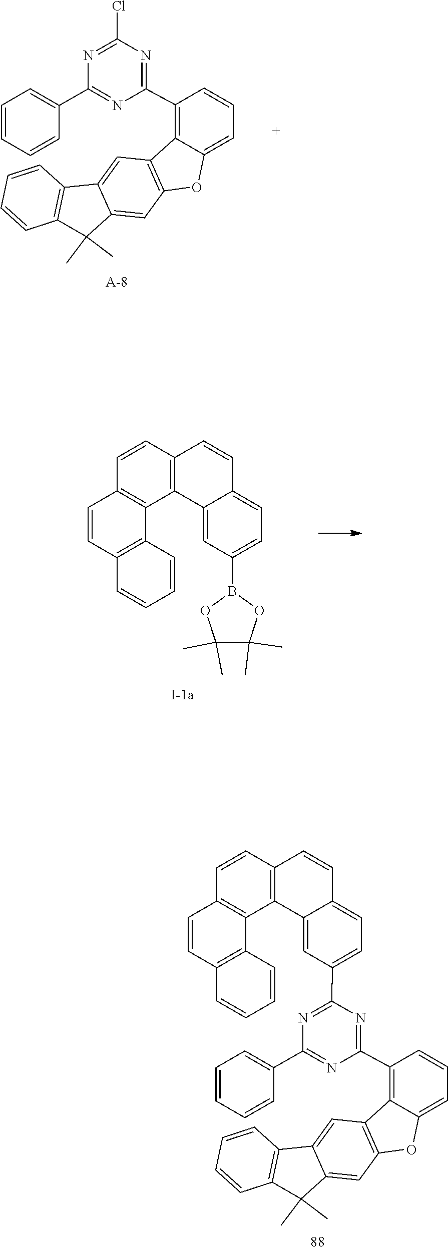

I-1a was synthesized according to the synthetic procedure described in Synthesis Examples 1-9 to 1-12, except that 4-bromophenanthrene was used instead of A-9a in Synthesis Example 1-9. Then, Compound 88 (yield 47.3%) was synthesized in the same manner as in Synthesis Example 1-13, except that I-1a was used instead of A-12.

MS (MALDI-TOF): m/z 715.26 [M⁺]

### Synthesis Example 10: Synthesis of Compound 89

### Synthesis Example 10-1: Synthesis of J-1

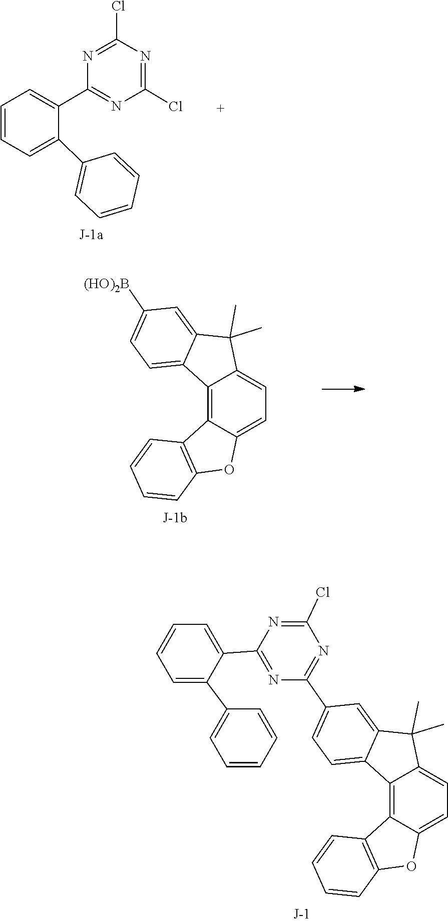 [SMILES: Clc1nc(Cl)nc(-c2ccccc2-c2ccccc2)n1 ; CC1(C)c2cc(B(O)O)ccc2-c2c1ccc1oc3ccccc3c21 ; CC1(C)c2cc(-c3nc(Cl)nc(-c4ccccc4-c4ccccc4)n3)ccc2-c2c1ccc1oc3ccccc3c21]

J-1b was synthesized with reference to Korean Patent Publication No. 10-2438615. Then, J-1 (yield 70.1%) was synthesized in the same manner as in Synthesis Example 1-8, except that J-1a and J-1b were used instead of A-8a and A-7, respectively.

### Synthesis Example 10-2: Synthesis of Compound 89

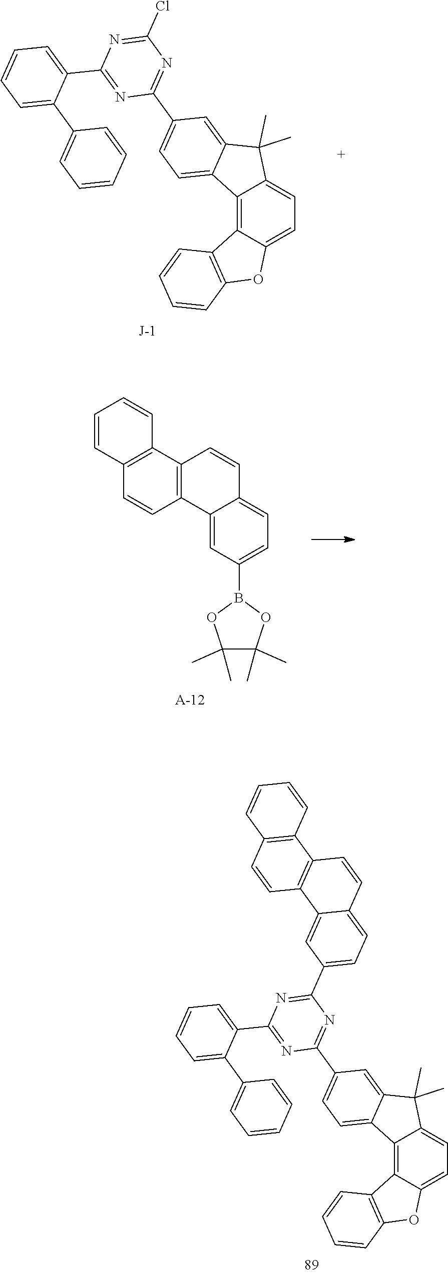

Compound 89 (yield 47.3%) was synthesized in the same manner as in Synthesis Example 1-13, except that J-1 was used instead of A-8.

MS (MALDI-TOF): m/z 741.28 [M⁺]

### Synthesis Example 11: Synthesis of Compound 97

### Synthesis Example 11-1: Synthesis of K-1

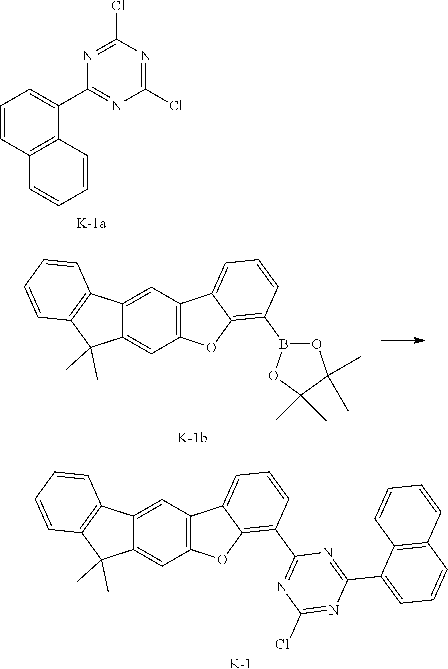

K-1b was synthesized with reference to Korean Patent Publication No. 10-2438615. Then, K-1 (yield 65.9%) was synthesized in the same manner as in Synthesis Example 1-8, except that K-1a and K-1b were used instead of A-8a and A-7, respectively.

### Synthesis Example 11-2: Synthesis of Compound 97

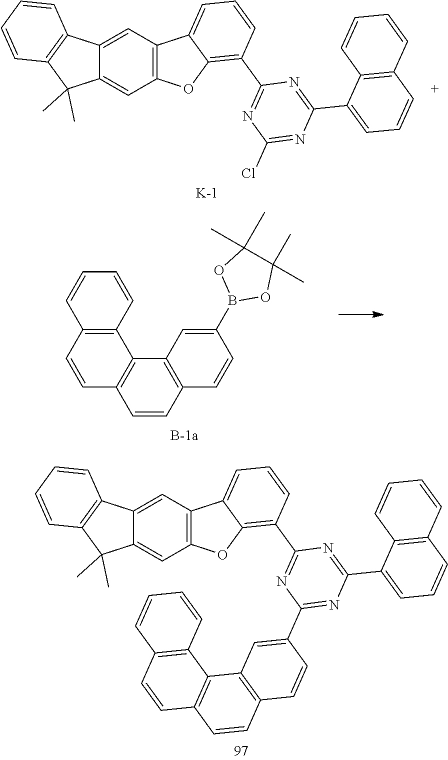

Compound 97 (yield 50%) was synthesized in the same manner as in Synthesis Example 1-13, except that K-1 and B-1a were used instead of A-8 and A-12, respectively.

MS (MALDI-TOF): m/z 715.26 [M⁺]

### Synthesis Example 12: Synthesis of Compound 98

### Synthesis Example 12-1: Synthesis of L-1

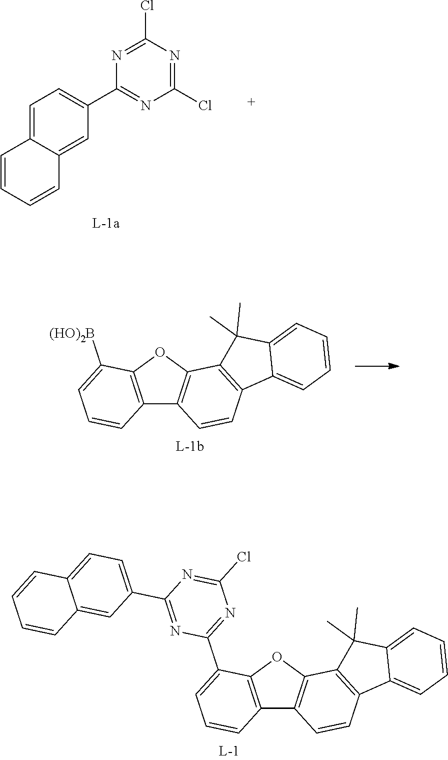 [SMILES: Clc1nc(Cl)nc(-c2ccc3ccccc3c2)n1 ; CC1(C)c2ccccc2-c2ccc3c(oc4c(B(O)O)cccc43)c21 ; CC1(C)c2ccccc2-c2ccc3c(oc4c(-c5nc(Cl)nc(-c6ccc7ccccc7c6)n5)cccc43)c21]

L-1b was synthesized with reference to Korean Patent Publication No. 10-2438615. Then, L-1 (yield 66.3%) was synthesized in the same manner as in Synthesis Example 1-8, except that K-1a and K-1b were used instead of A-8a and A-7, respectively.

### Synthesis Example 12-2: Synthesis of Compound 98

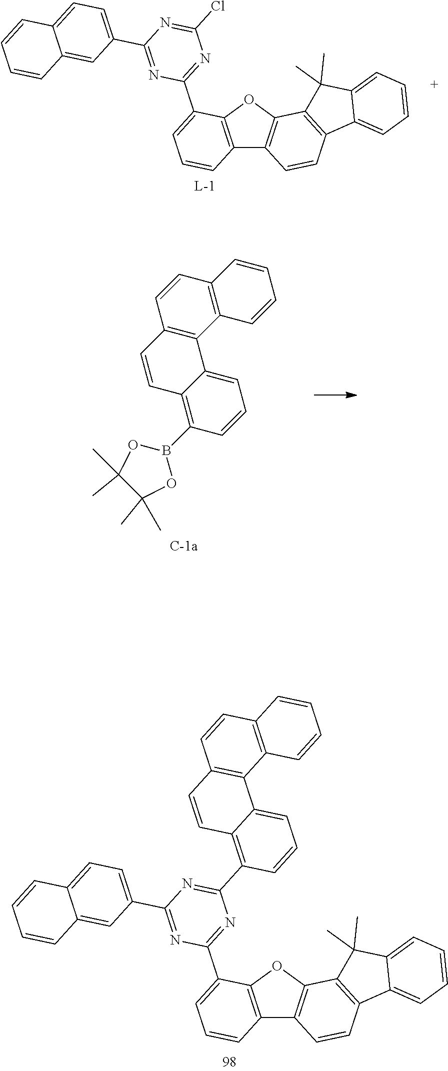 [SMILES: CC1(C)c2ccccc2-c2ccc3c(oc4c(-c5nc(Cl)nc(-c6ccc7ccccc7c6)n5)cccc43)c21 ; CC1(C)OB(c2cccc3c2ccc2ccc4ccccc4c23)OC1(C)C ; CC1(C)c2ccccc2-c2ccc3c(oc4c(-c5nc(-c6ccc7ccccc7c6)nc(-c6cccc7c6ccc6ccc8ccccc8c67)n5)cccc43)c21]

Compound 98 (yield 53.3%) was synthesized in the same manner as in Synthesis Example 1-13, except that L-1 and C-1a were used instead of A-8 and A-12, respectively.

MS (MALDI-TOF): m/z 715.26 [M⁺]

### Synthesis Example 13: Synthesis of Compound 99

### Synthesis Example 13-1: Synthesis of M-1

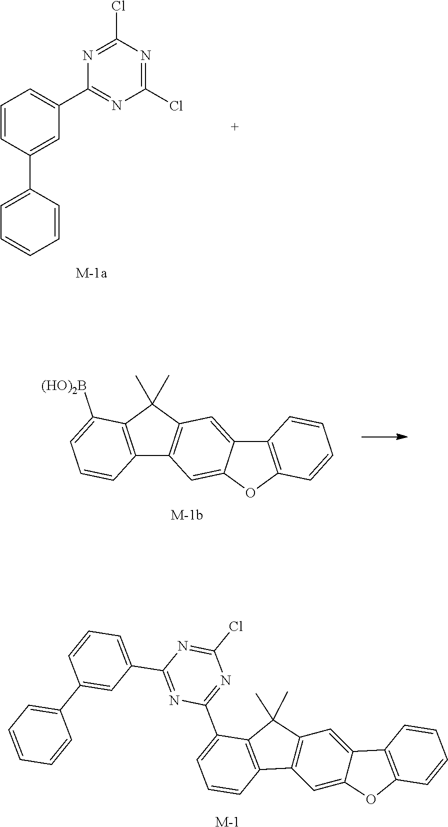

M-1b was synthesized with reference to Korean Patent Publication No. 10-2438615. Then, K-1 (yield 52.1%) was synthesized in the same manner as in Synthesis Example 1-8, except that M-1a and M-1b were used instead of A-8a and A-7, respectively.

### Synthesis Example 13-2: Synthesis of Compound 99

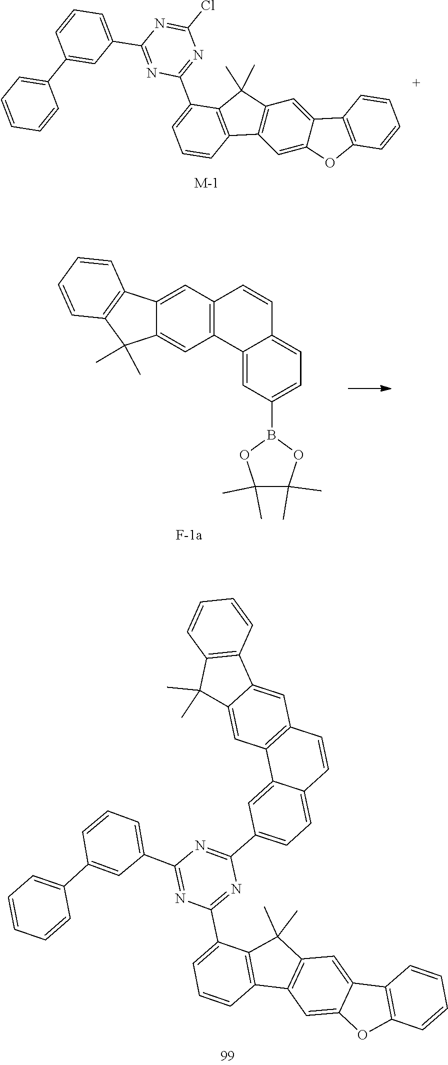

Compound 99 (yield 49.4%) was synthesized in the same manner as in Synthesis Example 1-13, except that M-1 and F-1a were used instead of A-8 and A-12, respectively.

MS (MALDI-TOF): m/z 807.32 [M⁺]

### Synthesis Example 14: Synthesis of Compound 106

### Synthesis Example 14-1: Synthesis of N-1

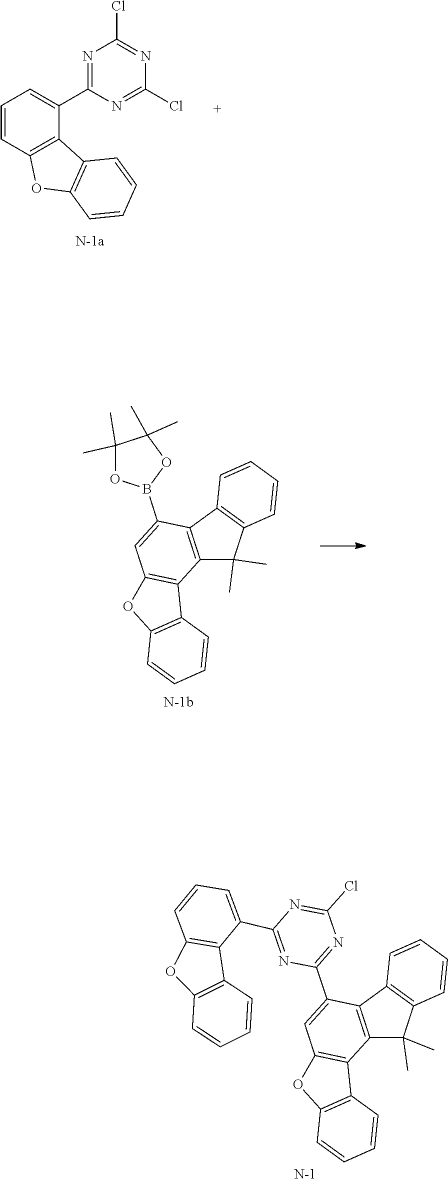

N-1b was synthesized with reference to Korean Patent Publication No. 10-2438615. Then, N-1 (yield 69.7%) was synthesized in the same manner as in Synthesis Example 1-8, except that N-1a and N-1b were used instead of A-8a and A-7, respectively.

### Synthesis Example 14-2: Synthesis of Compound 106

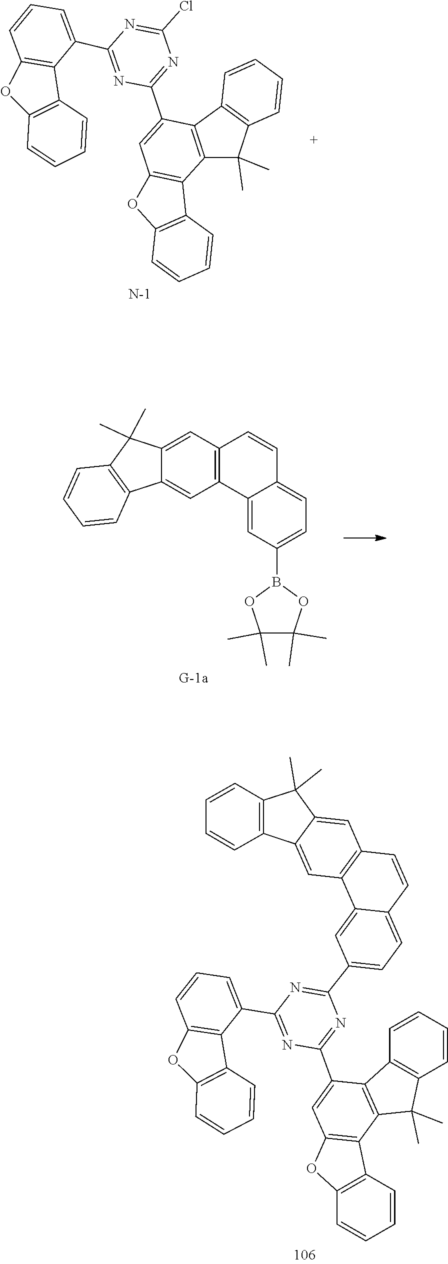

Compound 106 (yield 56.3%) was synthesized in the same manner as in Synthesis Example 1-13, except that N-1 and G-1a were used instead of A-8 and A-12, respectively.

MS (MALDI-TOF): m/z 821.30 [M⁺]

### Examples 1-14: Fabrication of Organic Light Emitting Devices

ITO glass was patterned to have a light emitting area of 2 mm×2 mm, followed by cleaning. After the cleaned ITO glass was mounted in a vacuum chamber, the base pressure was adjusted to 1×10⁻⁶ torr. HATCN (50 Å) and NPD (1500 Å) were deposited in this order on the ITO glass. The inventive compound shown in Table 1 as a host compound and RH were mixed in a 1:1 weight ratio. The mixture and RD (5 wt %) as a dopant compound were used to form a 400 Å thick light emitting layer. Thereafter, a mixture of ET and Liq in a ratio of 1:1 was used to form a 300 Å electron transport/injecting layer, and Liq (10 Å) and Al (1,000 Å) were sequentially deposited thereon to fabricate an organic light emitting device. The luminescent properties of the organic light emitting device were measured at 0.4 mA. The structures of HATCN, NPD, RD, RH, ET, and Liq are as follows:

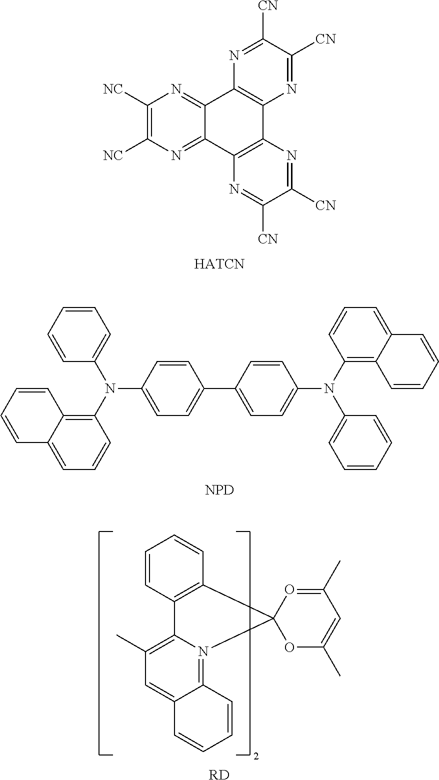

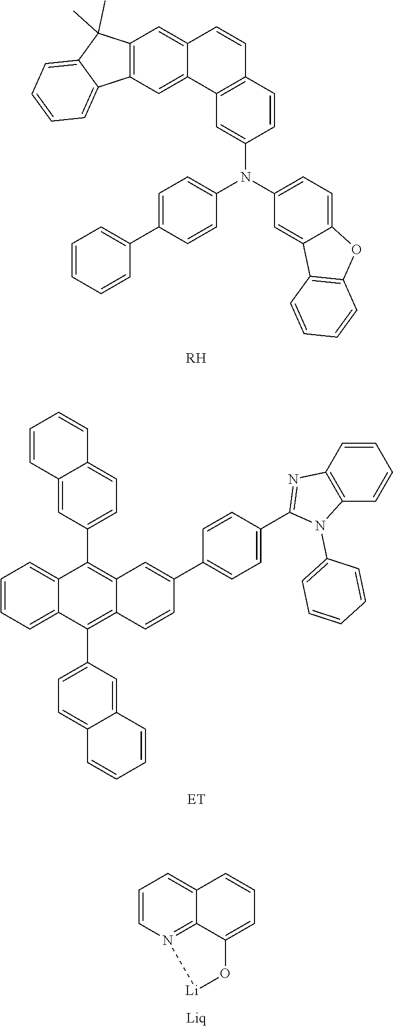

### Comparative Examples 1 and 2

Organic light emitting devices were fabricated in the same manner as in Examples 1-14, except that RH-A or RH—B was used as a host compound instead of the inventive compound. The luminescent properties of the organic light emitting devices were measured at 0.4 mA. The structures of RH-A and RH—B are as follow:

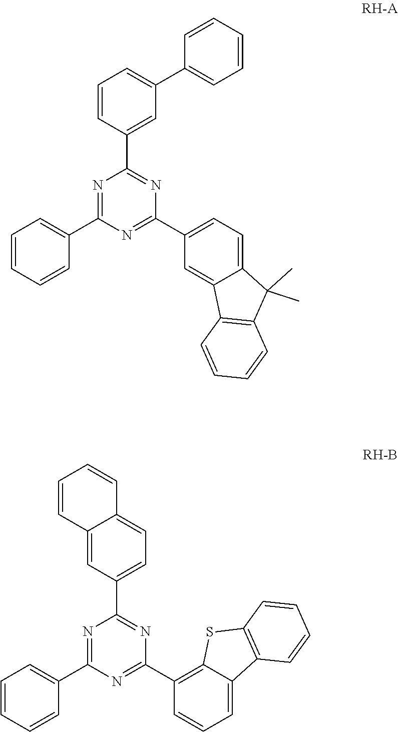 [SMILES: CC1(C)c2ccccc2-c2cc(-c3nc(-c4ccccc4)nc(-c4cccc(-c5ccccc5)c4)n3)ccc21 ; c1ccc(-c2nc(-c3ccc4ccccc4c3)nc(-c3cccc4c3sc3ccccc34)n2)cc1]

TABLE 1

| | | Driving | Current | | Color of |
|---|---|---|---|---|---|
| Example | | voltage | efficiency | Lifetime | light |
| No. | Host | (V) | (cd/A) | (T95, hr) | emission |
| Example 1 | Compound | 3.40 | 26.5 | 280 | Red |
| | 55 | | | | |
| Example 2 | Compound | 3.41 | 26.3 | 300 | Red |
| | 58 | | | | |
| Example 3 | Compound | 3.44 | 26.4 | 302 | Red |
| | 61 | | | | |
| Example 4 | Compound | 3.43 | 26.9 | 291 | Red |
| | 67 | | | | |
| Example 5 | Compound | 3.41 | 26.1 | 305 | Red |
| | 68 | | | | |
| Example 6 | Compound | 3.31 | 26.4 | 298 | Red |
| | 79 | | | | |
| Example 7 | Compound | 3.25 | 26.5 | 293 | Red |
| | 80 | | | | |
| Example 8 | Compound | 3.27 | 26.5 | 291 | Red |
| | 81 | | | | |
| Example 9 | Compound | 3.29 | 26.3 | 289 | Red |
| | 88 | | | | |
| Example 10 | Compound | 3.26 | 26.7 | 310 | Red |
| | 89 | | | | |
| Example 11 | Compound | 3.31 | 26.5 | 305 | Red |
| | 97 | | | | |
| Example 12 | Compound | 3.30 | 26.8 | 286 | Red |
| | 98 | | | | |
| Example 13 | Compound | 3.29 | 26.7 | 293 | Red |
| | 99 | | | | |
| Example 14 | Compound | 3.28 | 26.2 | 299 | Red |
| | 106 | | | | |
| Comparative | RH-A | 4.57 | 18.3 | 73 | Red |
| Example 1 | | | | | |
| Comparative | RH-B | 4.12 | 18.2 | 80 | Red |
| Example 2 | | | | | |

As can be seen from the results in Table 1, the organic light emitting devices of Examples 1-14, each of which employed the inventive host compound for the light emitting layer, had high quantum efficiencies, low driving voltages, and improved life characteristics compared to the organic light emitting devices of Comparative Examples 1-2, each of which employed the host compound whose structural features are contrasted with those of the inventive compounds. These results concluded that the use of the inventive compounds makes the organic light emitting devices highly efficient and long lasting.

What is claimed is:

1. An organic compound represented by Formula 1: 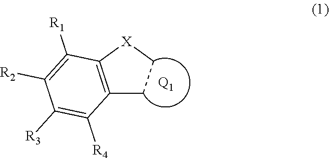
wherein X is O or S, Q₁ is 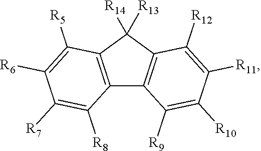
one of the pairs of the adjacent carbon atoms to which R₉ and R₁₀, R₁₀ and R₁₁, and R₁₁ and R₁₂ are attached is condensed with the pair of the adjacent carbon atoms connected by the dashed line form a ring, R₁₃ and R₁₄ are the same as or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and cyclic groups in which a substituted or unsubstituted C₃-C₃₀ aliphatic ring and a C₅-C₃₀ aromatic ring are fused together, with the proviso that R₁₃ and R₁₄ are optionally linked to each other to form an alicyclic or aromatic mono- or polycyclic ring, and the remainders of R₉ to R₁₂ that do not participate in the ring formation and R₁ to R₈ are the same as or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, cyclic groups in which a substituted or unsubstituted C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, with the proviso that each of R₁ to R₁₂ is optionally linked to an adjacent substituent to form an alicyclic or aromatic mono- or polycyclic ring and that at least one of R₁ to R₁₂ is linked to Structural Formula 1: 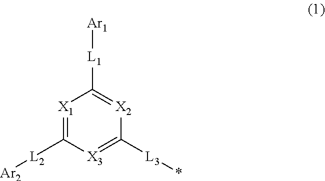
wherein * represents a site at which one of R₁ to R₁₂ and L₃ form a single bond, X₁ to X₃ are the same as or different from each other and are each independently CR or N, with the proviso that at least one of X₁ to X₃ is N, each R is independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and cyclic groups in which a substituted or unsubstituted C₃-C₃₀ aliphatic ring and a C₅-C₃₀ aromatic ring are fused together, L₁ to L₃ are the same as or different from each other and are each independently a single bond or selected from substituted or unsubstituted C₆-C₃₀ arylene, substituted or unsubstituted C₂-C₃₀ heteroarylene, and divalent cyclic groups in which a substituted or unsubstituted C₃-C₂₄ aliphatic ring and a C₅-C₂₄ aromatic ring are fused together, Ar₁ is selected from substituted or unsubstituted C₆-C₃₀ aryl, substituted or unsubstituted C₂-C₃₀ heteroaryl, and cyclic groups in which a substituted or unsubstituted C₃-C₂₄ aliphatic ring and a C₅-C₂₄ aromatic ring are fused together, and Ar₂ is represented by Structural Formula 2: 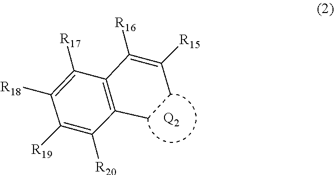
wherein Q₂ is represented by one of Structural Formulas A, B, and C: 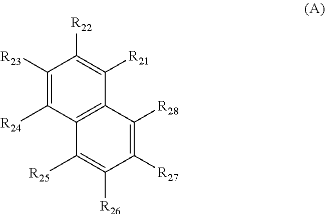
wherein one of the pairs of the adjacent carbon atoms to which R₂₁ and R₂₂, R₂₂ and R₂₃, and R₂₃ and R₂₄ are attached is condensed with the pair of the adjacent carbon atoms connected by the dashed line in Structural Formula 2 form a ring; 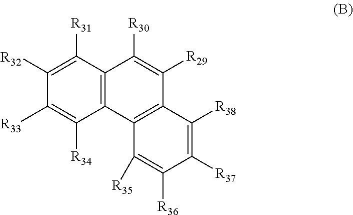
wherein one of the pairs of the adjacent carbon atoms to which R₂₉ and R₃₀, R₃₁ and R₃₂, R₃₂ and R₃₃, and R₃₃ and R₃₄ are attached is condensed with the pair of the adjacent carbon atoms connected by the dashed line in Structural Formula 2 form a ring; and 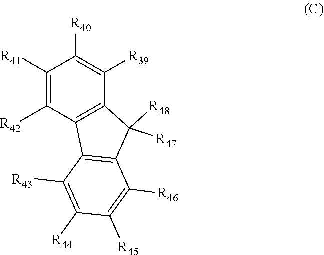
wherein one of the pairs of the adjacent carbon atoms to which R₃₉ and R₄₀, R₄₀ and R₄₁, and R₄₁ and R₄₂ are attached is condensed with the pair of the adjacent carbon atoms connected by the dashed line in Structural Formula 2 form a ring, R₄₇ and R₄₈ are the same as or different from each other and are each independently selected from hydrogen, deuterium, substituted or unsubstituted C₁-C₂₄ alkyl, substituted or unsubstituted C₂-C₂₄ alkynyl, substituted or unsubstituted C₂-C₂₄ alkenyl, substituted or unsubstituted C₆-C₃₀ aryl, substituted or unsubstituted C₃-C₃₀ cycloalkyl, substituted or unsubstituted C₂-C₃₀ heterocycloalkyl, substituted or unsubstituted C₂-C₃₀ heteroaryl, and cyclic groups in which a substituted or unsubstituted C₃-C₂₄ aliphatic ring and a C₅-C₂₄ aromatic ring are fused together, with the proviso that R₄₇ and R₄₈ are optionally linked to each other to form an alicyclic or aromatic mono- or polycyclic ring, the remainders of R₁₅ to R₄₆ that do not participate in the ring formation are the same as or different from each other and are each independently selected from hydrogen, deuterium, substituted or unsubstituted C₁-C₂₄ alkyl, substituted or unsubstituted C₂-C₂₄ alkynyl, substituted or unsubstituted C₂-C₂₄ alkenyl, substituted or unsubstituted C₆-C₃₀ aryl, substituted or unsubstituted C₃-C₃₀ cycloalkyl, substituted or unsubstituted C₂-C₃₀ heterocycloalkyl, substituted or unsubstituted C₂-C₃₀ heteroaryl, cyclic groups in which a substituted or unsubstituted C₃-C₂₄ aliphatic ring and a C₅-C₂₄ aromatic ring are fused together, substituted or unsubstituted C₁-C₂₄ alkoxy, substituted or unsubstituted C₆-C₂₄ aryloxy, substituted or unsubstituted C₁-C₂₄ alkylthioxy, substituted or unsubstituted C₅-C₂₄ arylthioxy, substituted or unsubstituted amine, substituted or unsubstituted silyl, substituted or unsubstituted germanium, nitro, cyano, and halogen, with the proviso that each of R₁₅ to R₄₈ is optionally linked to an adjacent substituent to form an alicyclic or aromatic mono- or polycyclic ring, the “substituted” in the definitions of R₁ to R₁₄ in Formula 1 and Structural Formulas 1, 2, A, B, and C indicating substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, cyclic groups in which a C₃-C₂₄ aliphatic ring and a C₃-C₂₄ aromatic ring are fused together, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof, the “unsubstituted” in the same definition indicating having no substituent, one or more hydrogen atoms in each of the substituents being optionally replaced by deuterium atoms, and two or more adjacent ones of the substituents being optionally linked to each other to form an alicyclic or aromatic mono- or polycyclic ring.

2. The organic compound according to claim 1, wherein Formula 1 is selected from Formulas 1-1 to 1-6: 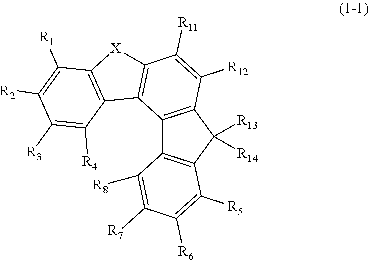
wherein R₁ to R₈, R₁₁ to R₁₄, and X are as defined in Formula 1: 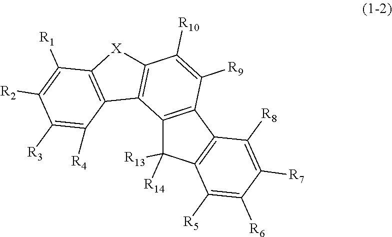
wherein R₁ to R₁₀, R₁₃, R₁₄, and X are as defined in Formula 1; 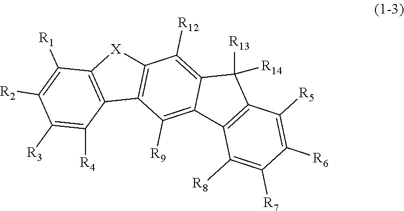
wherein R₁ to R₉, R₁₂ to R₁₄, and X are as defined in Formula 1; 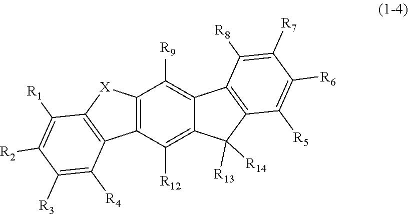
wherein R₁ to R₉, R₁₂ to R₁₄, and X are as defined in Formula 1; 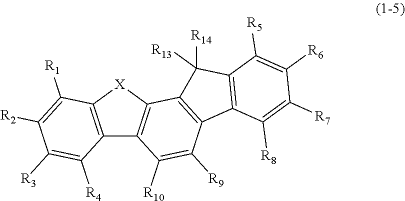
wherein R₁ to R₁₀, R₁₃, R₁₄, and X are as defined in Formula 1; and 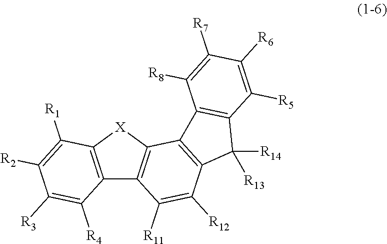
wherein R₁ to R₈, R₁₁ to R₁₄, and X are as defined in Formula 1.

3. The organic compound according to claim 1, wherein each of X₁ to X₃ in Structural Formula 1 is N.

4. The organic compound according to claim 1, wherein Structural Formula 2 is selected from Structural Formulas A-1 to A-3, B-1 to B-6, and C-1 to C-6: 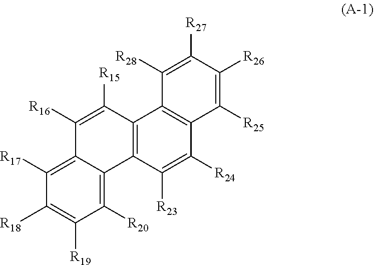
wherein R₁₅ to R₂₀ and R₂₃ to R₂₈ are as defined in Formula 1; 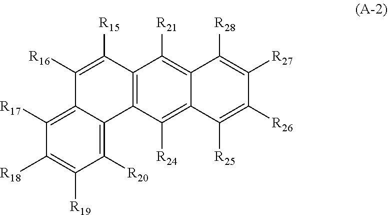
wherein R₁₅ to R₂₁ and R₂₄ to R₂₈ are as defined in Formula 1; 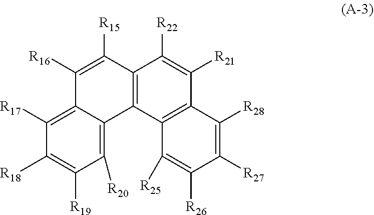
wherein R₁₅ to R₂₂ and R₂₅ to R₂₈ are as defined in Formula 1; 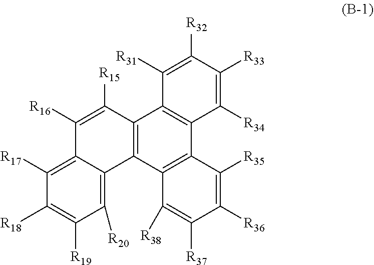
wherein R₁₅ to R₂₀ and R₃₁ to R₃₈ are as defined in Formula 1; 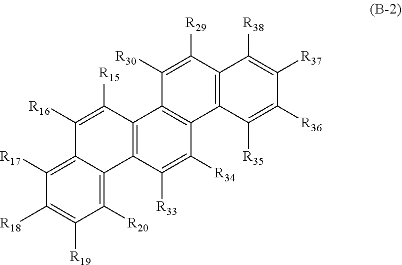
wherein R₁₅ to R₂₀, R₂₉, R₃₀, and R₃₃ to R₃₈ are as defined in Formula 1; 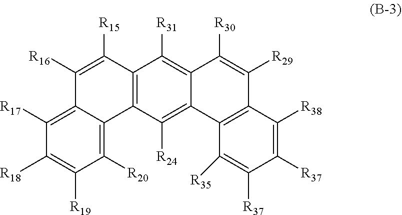
wherein R₁₅ to R₂₀, R₂₉ to R₃₁, and R₃₄ to R₃₈ are as defined in Formula 1; 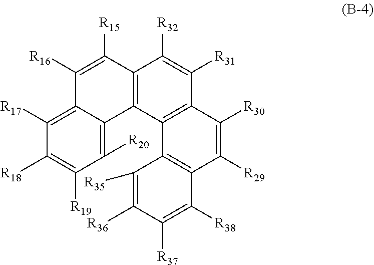
wherein R₁₅ to R₂₀, R₂₉ to R₃₂, and R₃₅ to R₃₈ are as defined in Formula 1; 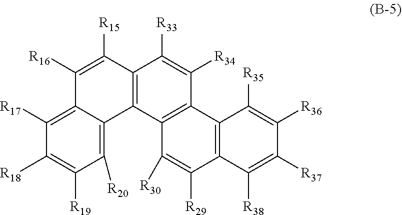
wherein R₁₅ to R₂₀, R₂₉, R₃₀, and R₃₃ to R₃₈ are as defined in Formula 1; 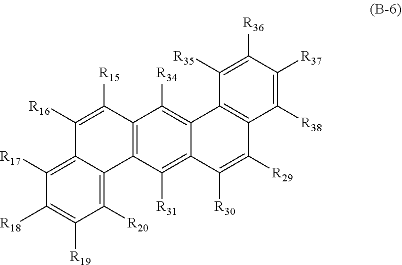
wherein R₁₅ to R₂₀, R₂₉ to R₃₁, and R₃₄ to R₃₈ are as defined in Formula 1; 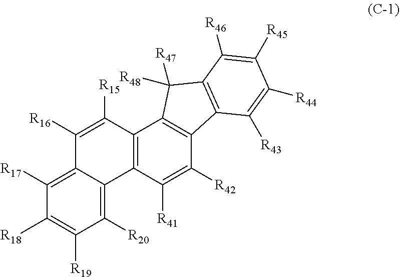
wherein R₁₅ to R₂₀ and R₄₁ to R₄₈ are as defined in Formula 1; 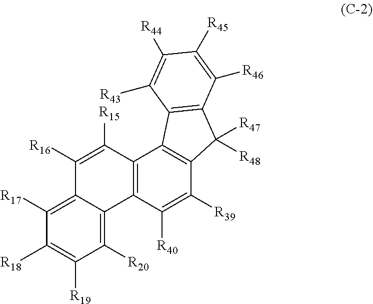
wherein R₁₅ to R₂₀, R₃₉, R₄₀, and R₄₃ to R₄₅ are as defined in Formula 1; 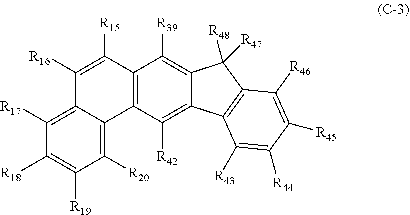
wherein R₁₅ to R₂₀, R₃₉, and R₄₂ to R₄₈ are as defined in Formula 1; 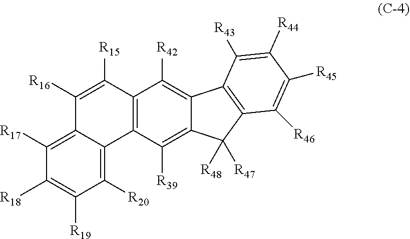
wherein R₁₅ to R₂₀, R₃₉, and R₄₂ to R₄₈ are as defined in Formula 1; 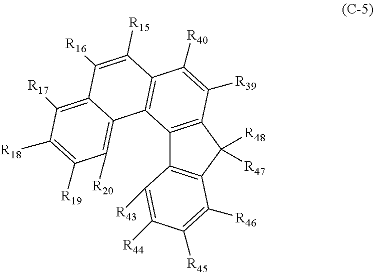
wherein R₁₅ to R₂₀, R₃₉, R₄₀, and R₄₃ to R₄₈ are as defined in Formula 1; and 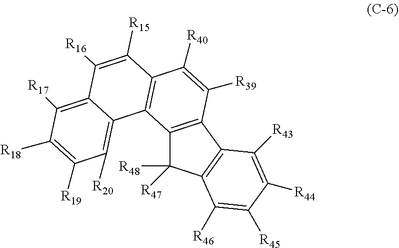
wherein R₁₅ to R₂₀ and R₄₁ to R₄₈ are as defined in Formula 1.

5. The organic compound according to claim 1, wherein the compound represented by Formula 1 is selected from the following compounds 1 to 180:

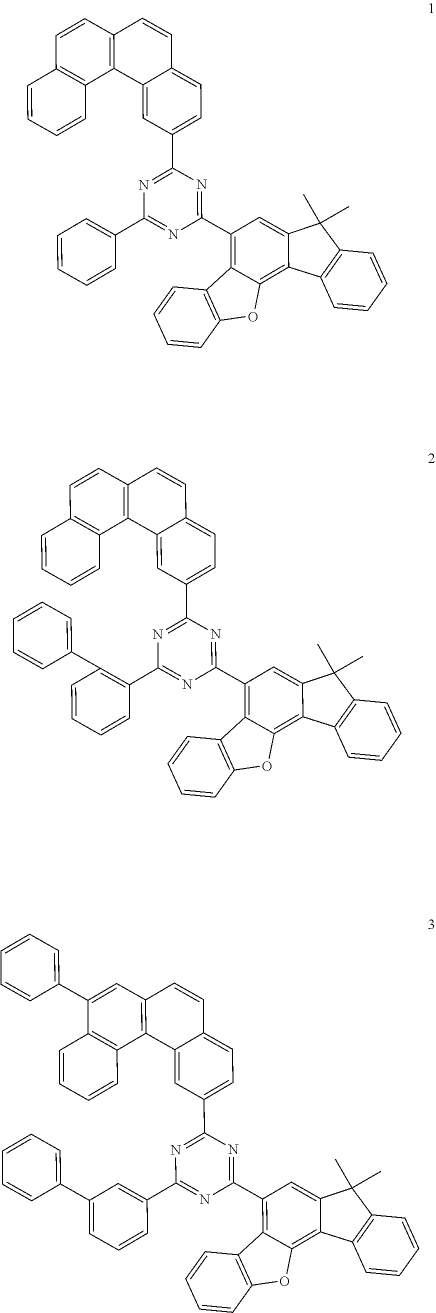 [SMILES: CC1(C)c2ccccc2-c2c1cc(-c1nc(-c3ccccc3)nc(-c3ccc4ccc5ccc6ccccc6c5c4c3)n1)c1c2oc2ccccc21 ; CC1(C)c2ccccc2-c2c1cc(-c1nc(-c3ccc4ccc5ccc6ccccc6c5c4c3)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5cc(-c6ccccc6)c6ccccc6c5c4c3)n1)c1c2oc2ccccc21]

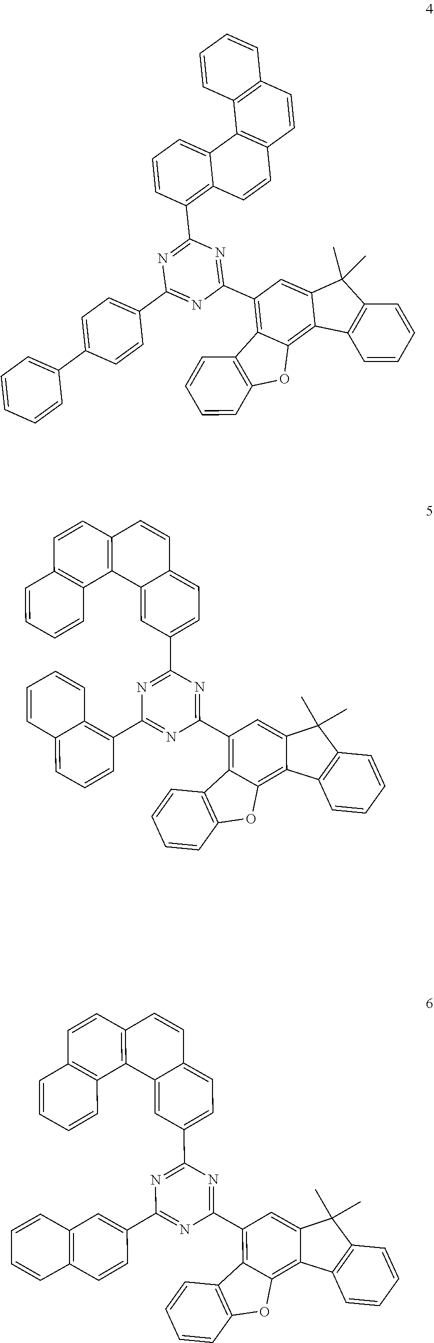 [SMILES: CC1(C)c2ccccc2-c2c1cc(-c1nc(-c3ccc(-c4ccccc4)cc3)nc(-c3cccc4c3ccc3ccc5ccccc5c34)n1)c1c2oc2ccccc21 ; CC1(C)c2ccccc2-c2c1cc(-c1nc(-c3ccc4ccc5ccc6ccccc6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6ccccc6c5c4c3)n1)c1c2oc2ccccc21]

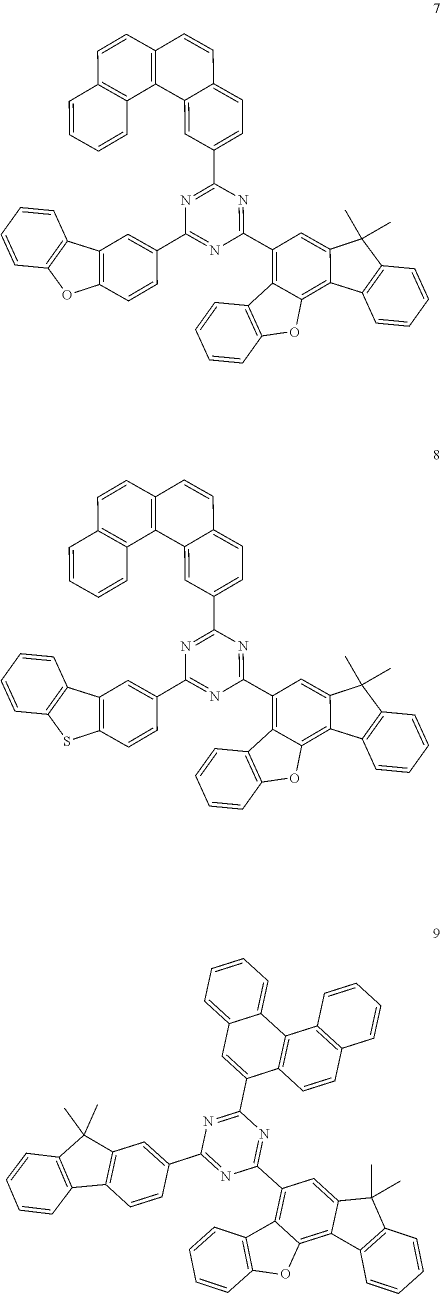 [SMILES: CC1(C)c2ccccc2-c2c1cc(-c1nc(-c3ccc4oc5ccccc5c4c3)nc(-c3ccc4ccc5ccc6ccccc6c5c4c3)n1)c1c2oc2ccccc21 ; CC1(C)c2ccccc2-c2c1cc(-c1nc(-c3ccc4sc5ccccc5c4c3)nc(-c3ccc4ccc5ccc6ccccc6c5c4c3)n1)c1c2oc2ccccc21 ; CC1(C)c2ccccc2-c2ccc(-c3nc(-c4cc5ccccc5c5c4ccc4ccccc45)nc(-c4cc5c(c6oc7ccccc7c46)-c4ccccc4C5(C)C)n3)cc21]

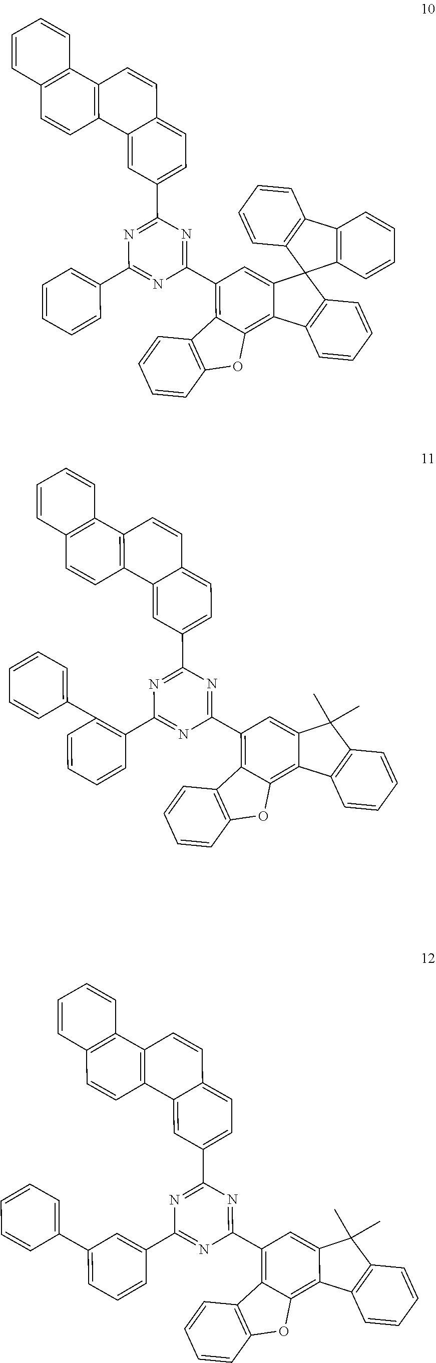 [SMILES: c1ccc(-c2nc(-c3ccc4ccc5c6ccccc6ccc5c4c3)nc(-c3cc4c(c5oc6ccccc6c35)-c3ccccc3C43c4ccccc4-c4ccccc43)n2)cc1 ; CC1(C)c2ccccc2-c2c1cc(-c1nc(-c3ccc4ccc5c6ccccc6ccc5c4c3)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5c6ccccc6ccc5c4c3)n1)c1c2oc2ccccc21]

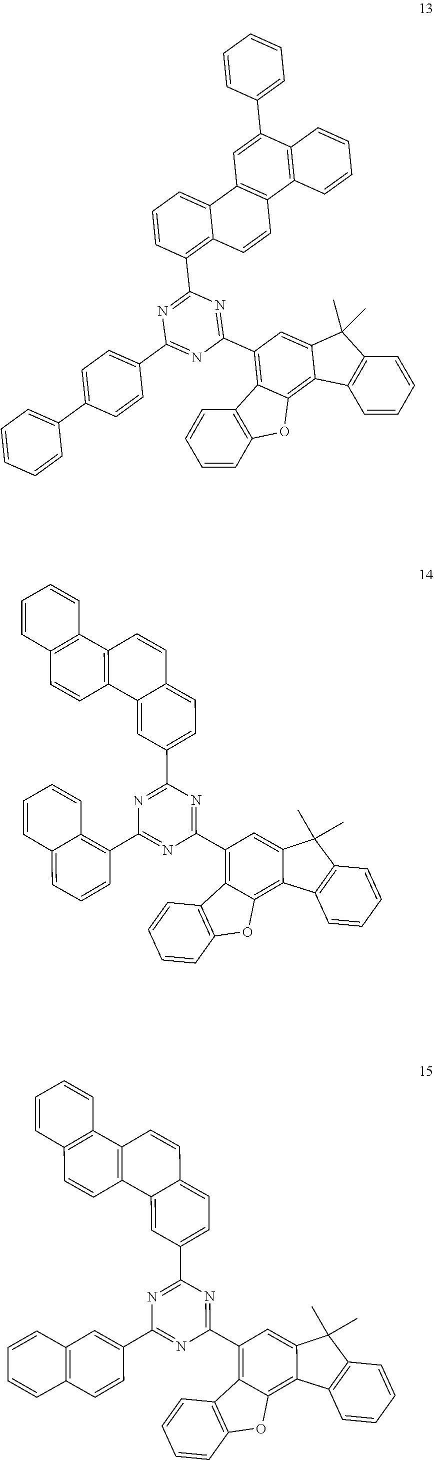 [SMILES: CC1(C)c2ccccc2-c2c1cc(-c1nc(-c3ccc(-c4ccccc4)cc3)nc(-c3cccc4c3ccc3c5ccccc5c(-c5ccccc5)cc43)n1)c1c2oc2ccccc21 ; CC1(C)c2ccccc2-c2c1cc(-c1nc(-c3ccc4ccc5c6ccccc6ccc5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5c6ccccc6ccc5c4c3)n1)c1c2oc2ccccc21]

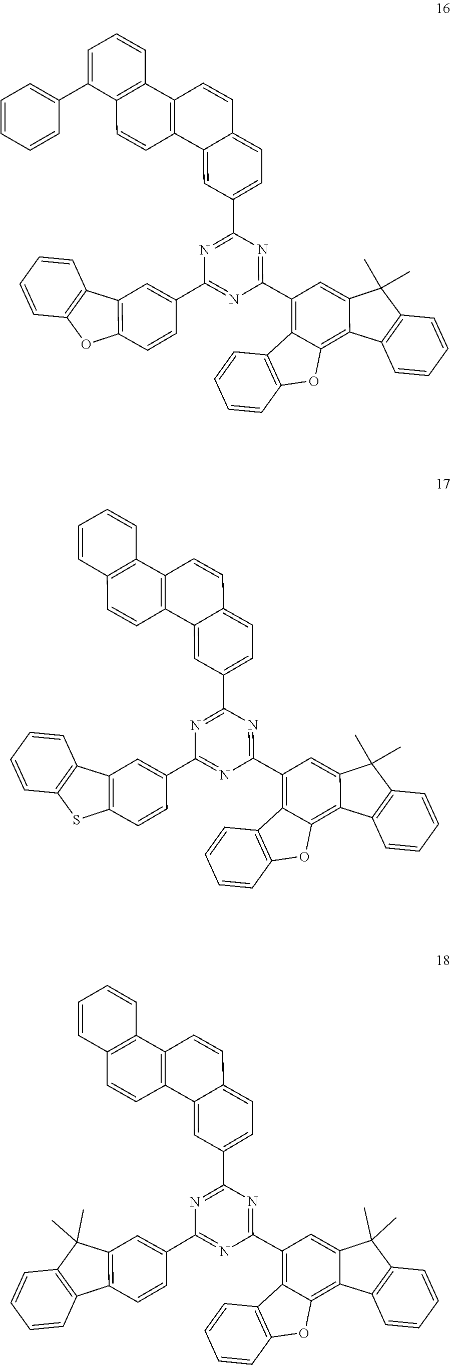 [SMILES: CC1(C)c2ccccc2-c2c1cc(-c1nc(-c3ccc4ccc5c6cccc(-c7ccccc7)c6ccc5c4c3)nc(-c3ccc4oc5ccccc5c4c3)n1)c1c2oc2ccccc21 ; CC1(C)c2ccccc2-c2c1cc(-c1nc(-c3ccc4ccc5c6ccccc6ccc5c4c3)nc(-c3ccc4sc5ccccc5c4c3)n1)c1c2oc2ccccc21 ; CC1(C)c2ccccc2-c2ccc(-c3nc(-c4ccc5ccc6c7ccccc7ccc6c5c4)nc(-c4cc5c(c6oc7ccccc7c46)-c4ccccc4C5(C)C)n3)cc21]

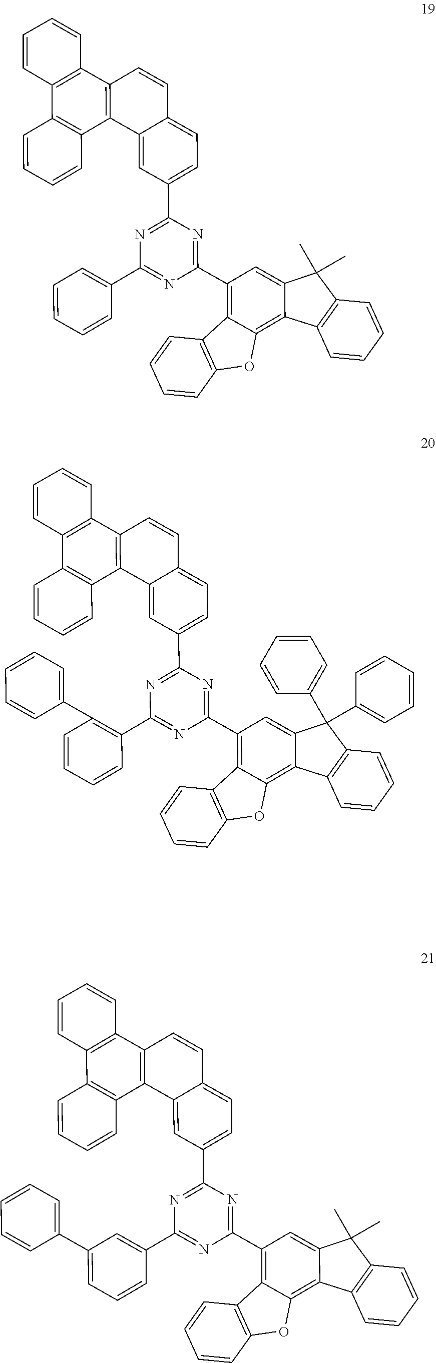 [SMILES: CC1(C)c2ccccc2-c2c1cc(-c1nc(-c3ccccc3)nc(-c3ccc4ccc5c6ccccc6c6ccccc6c5c4c3)n1)c1c2oc2ccccc21 ; c1ccc(-c2ccccc2-c2nc(-c3ccc4ccc5c6ccccc6c6ccccc6c5c4c3)nc(-c3cc4c(c5oc6ccccc6c35)-c3ccccc3C4(c3ccccc3)c3ccccc3)n2)cc1 ; CC1(C)c2ccccc2-c2c1cc(-c1nc(-c3cccc(-c4ccccc4)c3)nc(-c3ccc4ccc5c6ccccc6c6ccccc6c5c4c3)n1)c1c2oc2ccccc21]

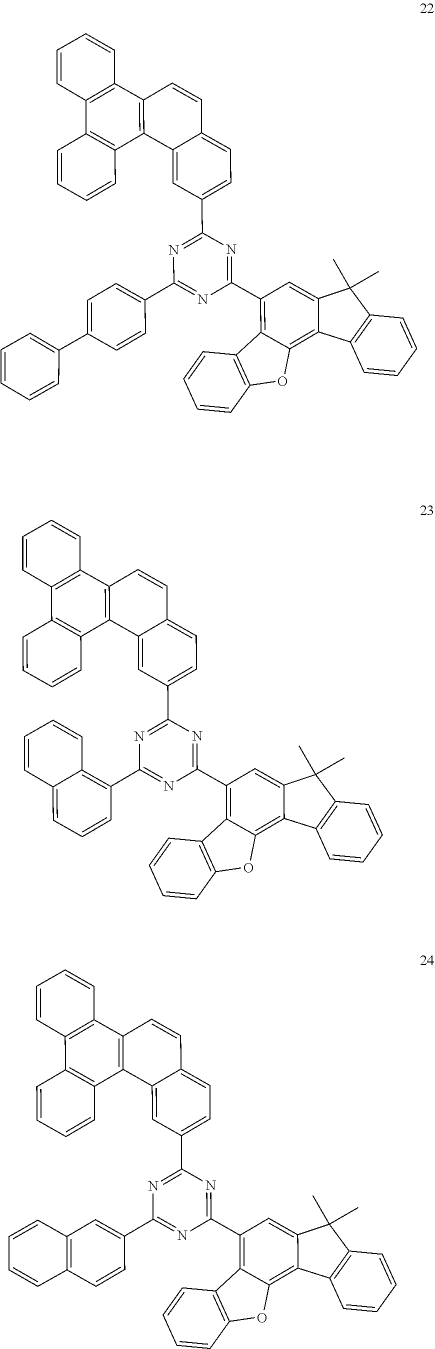 [SMILES: CC1(C)c2ccccc2-c2c1cc(-c1nc(-c3ccc(-c4ccccc4)cc3)nc(-c3ccc4ccc5c6ccccc6c6ccccc6c5c4c3)n1)c1c2oc2ccccc21 ; CC1(C)c2ccccc2-c2c1cc(-c1nc(-c3ccc4ccc5c6ccccc6c6ccccc6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5c6ccccc6c6ccccc6c5c4c3)n1)c1c2oc2ccccc21]

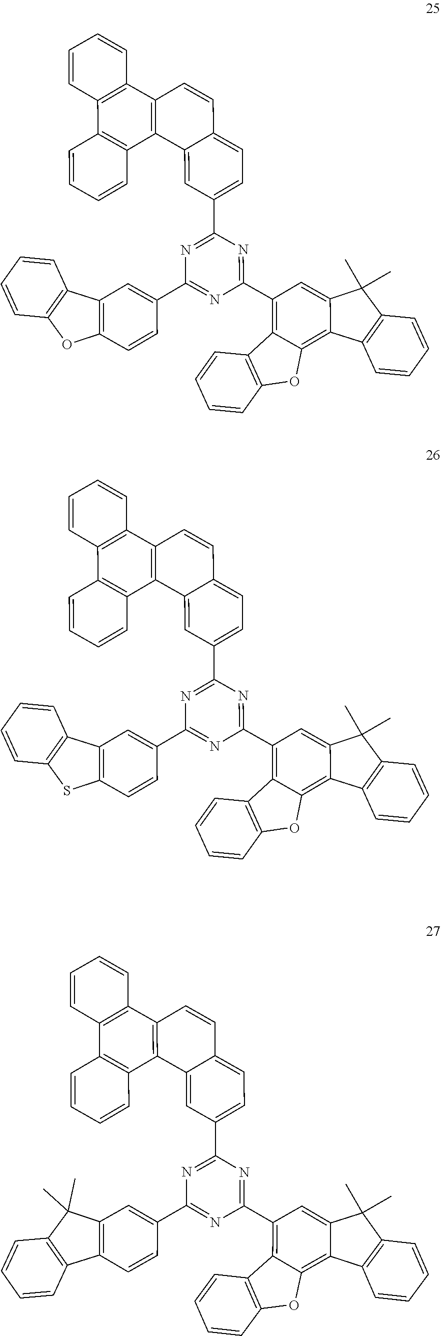 [SMILES: CC1(C)c2ccccc2-c2c1cc(-c1nc(-c3ccc4oc5ccccc5c4c3)nc(-c3ccc4ccc5c6ccccc6c6ccccc6c5c4c3)n1)c1c2oc2ccccc21 ; CC1(C)c2ccccc2-c2c1cc(-c1nc(-c3ccc4sc5ccccc5c4c3)nc(-c3ccc4ccc5c6ccccc6c6ccccc6c5c4c3)n1)c1c2oc2ccccc21 ; CC1(C)c2ccccc2-c2ccc(-c3nc(-c4ccc5ccc6c7ccccc7c7ccccc7c6c5c4)nc(-c4cc5c(c6oc7ccccc7c46)-c4ccccc4C5(C)C)n3)cc21]

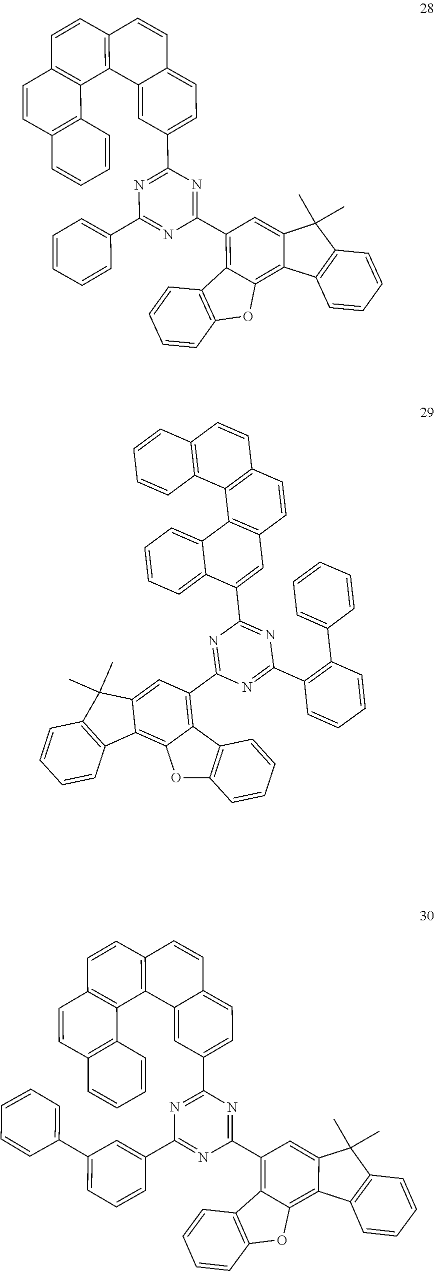 [SMILES: CC1(C)c2ccccc2-c2c1cc(-c1nc(-c3ccccc3)nc(-c3ccc4ccc5ccc6ccc7ccccc7c6c5c4c3)n1)c1c2oc2ccccc21 ; CC1(C)c2ccccc2-c2c1cc(-c1nc(-c3ccccc3-c3ccccc3)nc(-c3cc4ccc5ccc6ccccc6c5c4c4ccccc34)n1)c1c2oc2ccccc21 ; CC1(C)c2ccccc2-c2c1cc(-c1nc(-c3cccc(-c4ccccc4)c3)nc(-c3ccc4ccc5ccc6ccc7ccccc7c6c5c4c3)n1)c1c2oc2ccccc21]

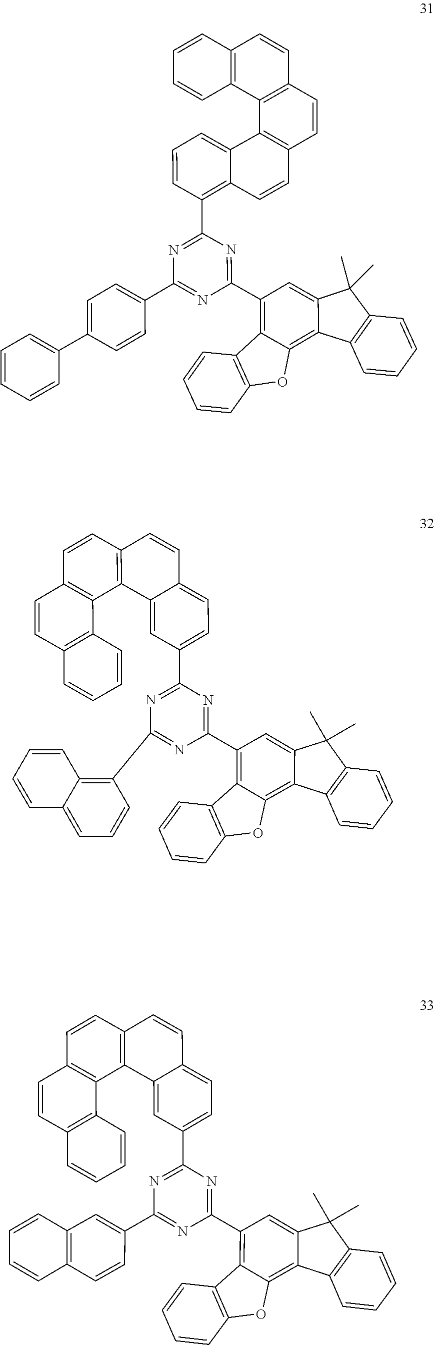 [SMILES: CC1(C)c2ccccc2-c2c1cc(-c1nc(-c3ccc(-c4ccccc4)cc3)nc(-c3cccc4c3ccc3ccc5ccc6ccccc6c5c34)n1)c1c2oc2ccccc21 ; CC1(C)c2ccccc2-c2c1cc(-c1nc(-c3ccc4ccc5ccc6ccc7ccccc7c6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6ccc7ccccc7c6c5c4c3)n1)c1c2oc2ccccc21]

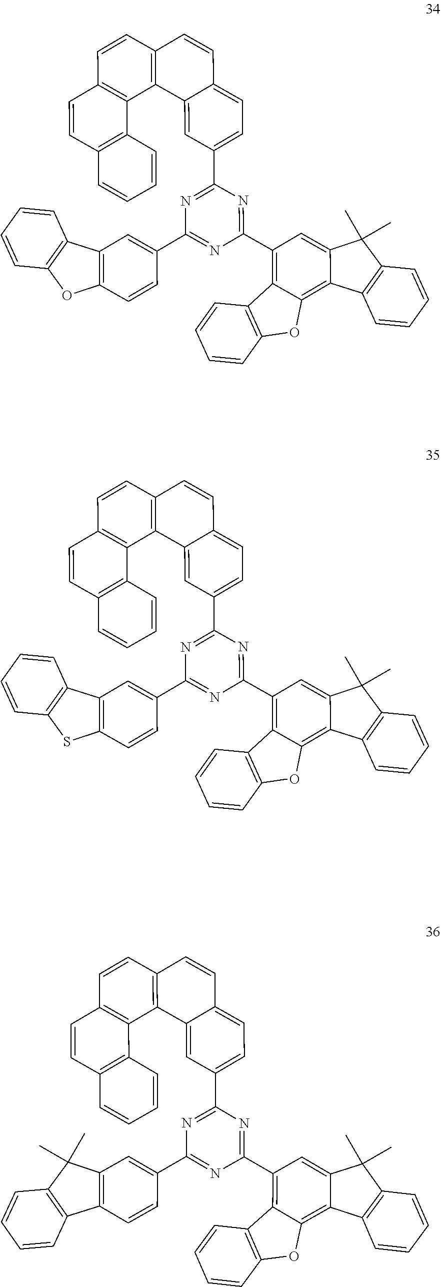 [SMILES: CC1(C)c2ccccc2-c2c1cc(-c1nc(-c3ccc4oc5ccccc5c4c3)nc(-c3ccc4ccc5ccc6ccc7ccccc7c6c5c4c3)n1)c1c2oc2ccccc21 ; CC1(C)c2ccccc2-c2c1cc(-c1nc(-c3ccc4sc5ccccc5c4c3)nc(-c3ccc4ccc5ccc6ccc7ccccc7c6c5c4c3)n1)c1c2oc2ccccc21 ; CC1(C)c2ccccc2-c2ccc(-c3nc(-c4ccc5ccc6ccc7ccc8ccccc8c7c6c5c4)nc(-c4cc5c(c6oc7ccccc7c46)-c4ccccc4C5(C)C)n3)cc21]

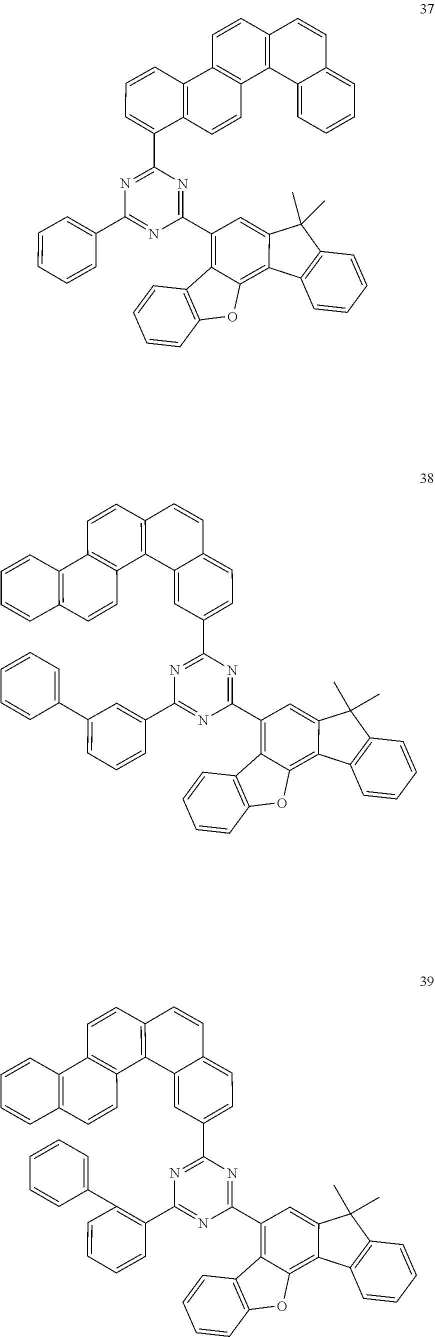 [SMILES: CC1(C)c2ccccc2-c2c1cc(-c1nc(-c3ccccc3)nc(-c3cccc4c3ccc3c4ccc4ccc5ccccc5c43)n1)c1c2oc2ccccc21 ; CC1(C)c2ccccc2-c2c1cc(-c1nc(-c3cccc(-c4ccccc4)c3)nc(-c3ccc4ccc5ccc6c7ccccc7ccc6c5c4c3)n1)c1c2oc2ccccc21 ; CC1(C)c2ccccc2-c2c1cc(-c1nc(-c3ccc4ccc5ccc6c7ccccc7ccc6c5c4c3)nc(-c3ccccc3-c3ccccc3)n1)c1c2oc2ccccc21]

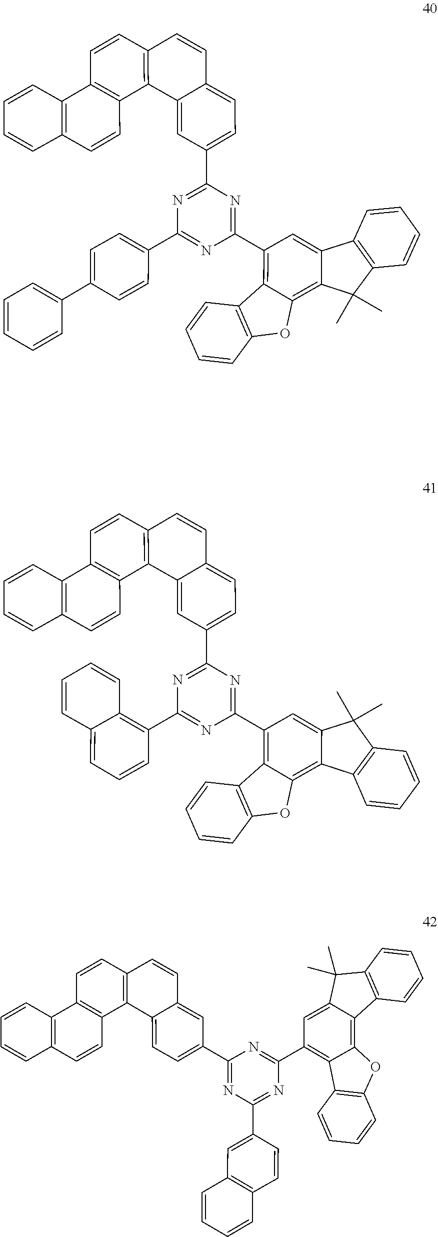 [SMILES: CC1(C)c2ccccc2-c2cc(-c3nc(-c4ccc(-c5ccccc5)cc4)nc(-c4ccc5ccc6ccc7c8ccccc8ccc7c6c5c4)n3)c3c(oc4ccccc43)c21 ; CC1(C)c2ccccc2-c2c1cc(-c1nc(-c3ccc4ccc5ccc6c7ccccc7ccc6c5c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4c(ccc5ccc6c7ccccc7ccc6c54)c3)n1)c1c2oc2ccccc21]

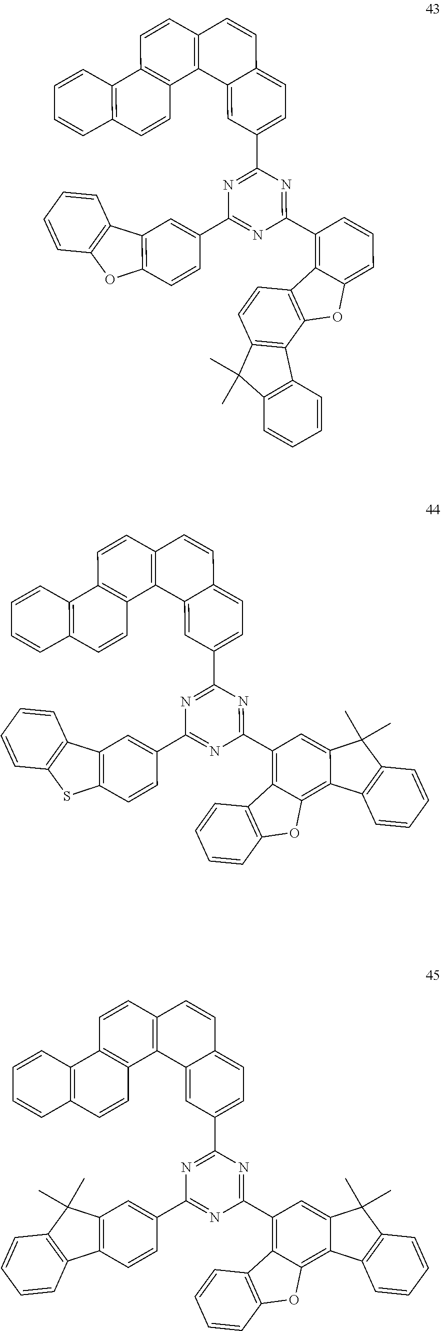 [SMILES: CC1(C)c2ccccc2-c2c1ccc1c2oc2cccc(-c3nc(-c4ccc5oc6ccccc6c5c4)nc(-c4ccc5ccc6ccc7c8ccccc8ccc7c6c5c4)n3)c21 ; CC1(C)c2ccccc2-c2c1cc(-c1nc(-c3ccc4sc5ccccc5c4c3)nc(-c3ccc4ccc5ccc6c7ccccc7ccc6c5c4c3)n1)c1c2oc2ccccc21 ; CC1(C)c2ccccc2-c2ccc(-c3nc(-c4ccc5ccc6ccc7c8ccccc8ccc7c6c5c4)nc(-c4cc5c(c6oc7ccccc7c46)-c4ccccc4C5(C)C)n3)cc21]

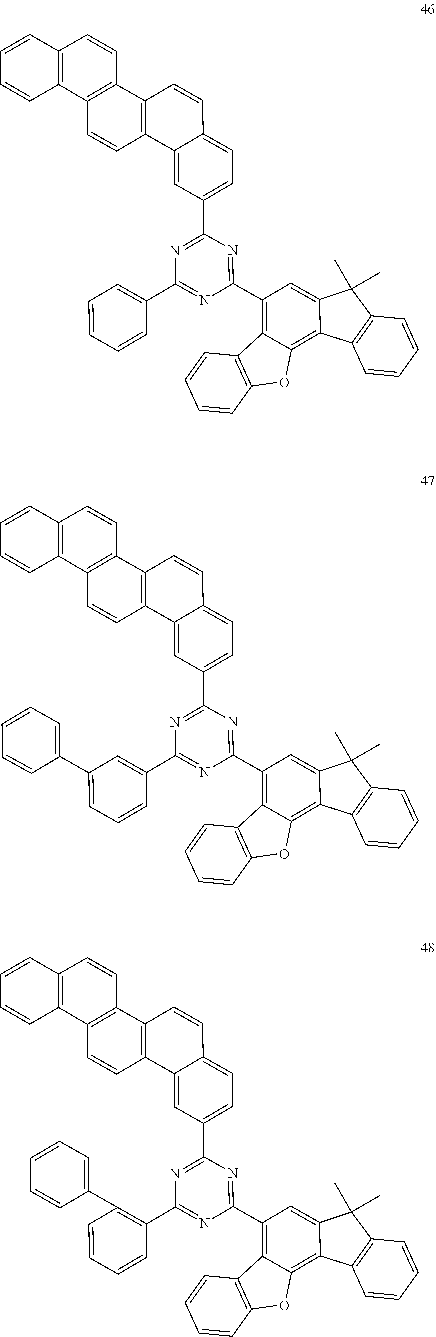 [SMILES: CC1(C)c2ccccc2-c2c1cc(-c1nc(-c3ccccc3)nc(-c3ccc4ccc5c(ccc6c7ccccc7ccc65)c4c3)n1)c1c2oc2ccccc21 ; CC1(C)c2ccccc2-c2c1cc(-c1nc(-c3cccc(-c4ccccc4)c3)nc(-c3ccc4ccc5c(ccc6c7ccccc7ccc65)c4c3)n1)c1c2oc2ccccc21 ; CC1(C)c2ccccc2-c2c1cc(-c1nc(-c3ccc4ccc5c(ccc6c7ccccc7ccc65)c4c3)nc(-c3ccccc3-c3ccccc3)n1)c1c2oc2ccccc21]

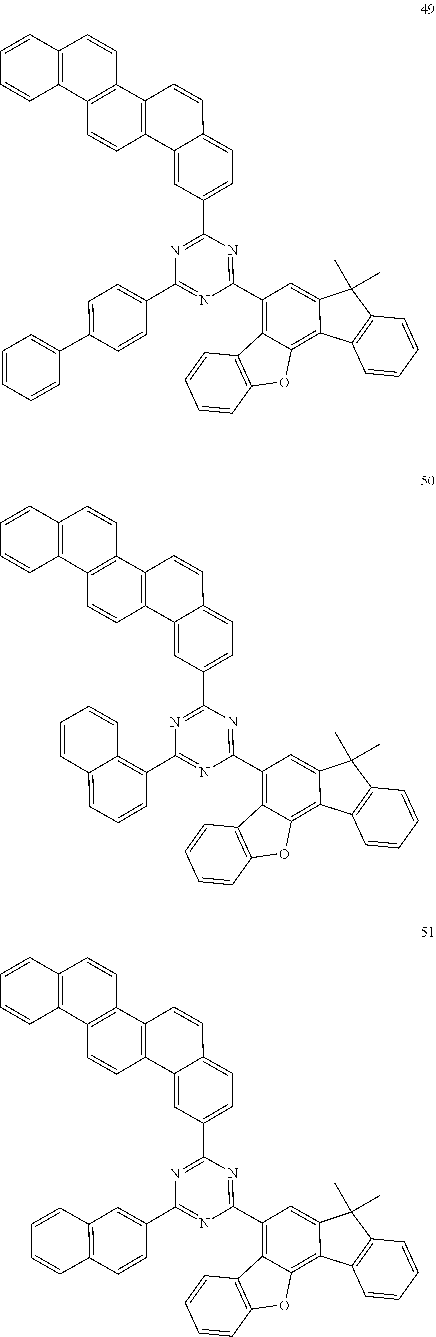 [SMILES: CC1(C)c2ccccc2-c2c1cc(-c1nc(-c3ccc(-c4ccccc4)cc3)nc(-c3ccc4ccc5c(ccc6c7ccccc7ccc65)c4c3)n1)c1c2oc2ccccc21 ; CC1(C)c2ccccc2-c2c1cc(-c1nc(-c3ccc4ccc5c(ccc6c7ccccc7ccc65)c4c3)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5c(ccc6c7ccccc7ccc65)c4c3)n1)c1c2oc2ccccc21]

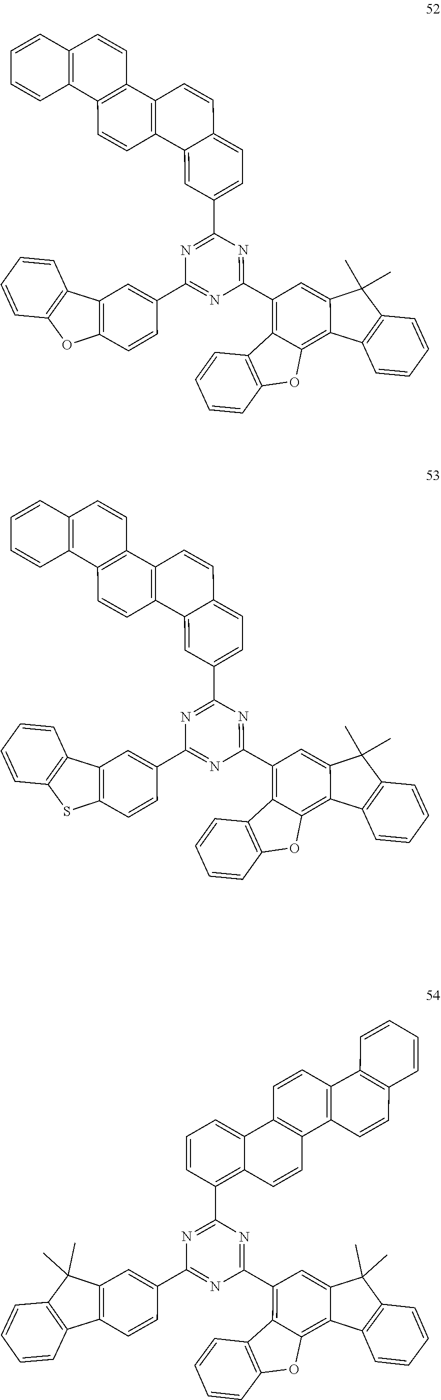 [SMILES: CC1(C)c2ccccc2-c2c1cc(-c1nc(-c3ccc4ccc5c(ccc6c7ccccc7ccc65)c4c3)nc(-c3ccc4oc5ccccc5c4c3)n1)c1c2oc2ccccc21 ; CC1(C)c2ccccc2-c2c1cc(-c1nc(-c3ccc4ccc5c(ccc6c7ccccc7ccc65)c4c3)nc(-c3ccc4sc5ccccc5c4c3)n1)c1c2oc2ccccc21 ; CC1(C)c2ccccc2-c2ccc(-c3nc(-c4cccc5c4ccc4c5ccc5c6ccccc6ccc54)nc(-c4cc5c(c6oc7ccccc7c46)-c4ccccc4C5(C)C)n3)cc21]

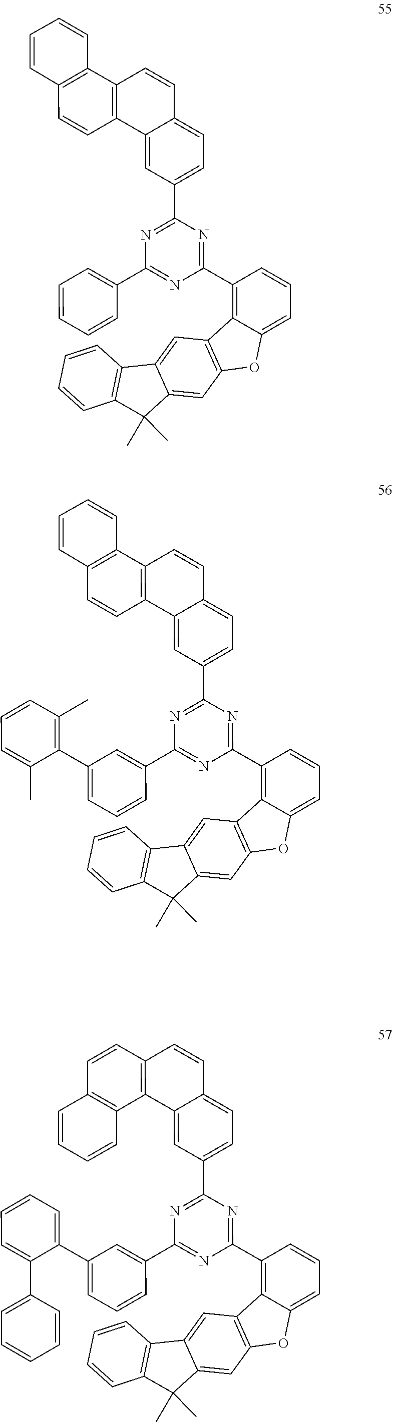 [SMILES: CC1(C)c2ccccc2-c2cc3c(cc21)oc1cccc(-c2nc(-c4ccccc4)nc(-c4ccc5ccc6c7ccccc7ccc6c5c4)n2)c13 ; Cc1cccc(C)c1-c1cccc(-c2nc(-c3ccc4ccc5c6ccccc6ccc5c4c3)nc(-c3cccc4oc5cc6c(cc5c34)-c3ccccc3C6(C)C)n2)c1 ; CC1(C)c2ccccc2-c2cc3c(cc21)oc1cccc(-c2nc(-c4cccc(-c5ccccc5-c5ccccc5)c4)nc(-c4ccc5ccc6ccc7ccccc7c6c5c4)n2)c13]

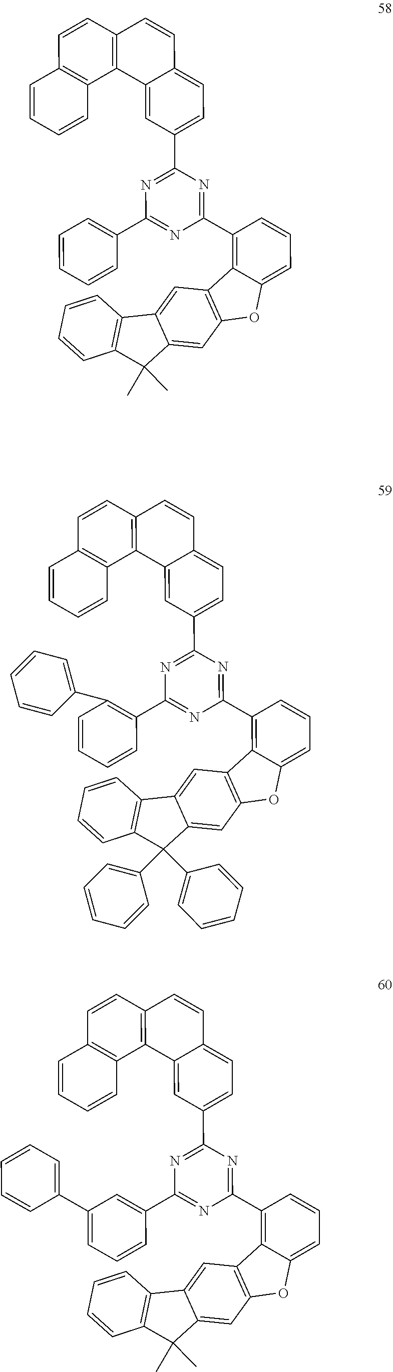 [SMILES: CC1(C)c2ccccc2-c2cc3c(cc21)oc1cccc(-c2nc(-c4ccccc4)nc(-c4ccc5ccc6ccc7ccccc7c6c5c4)n2)c13 ; c1ccc(-c2ccccc2-c2nc(-c3ccc4ccc5ccc6ccccc6c5c4c3)nc(-c3cccc4oc5cc6c(cc5c34)-c3ccccc3C6(c3ccccc3)c3ccccc3)n2)cc1 ; CC1(C)c2ccccc2-c2cc3c(cc21)oc1cccc(-c2nc(-c4cccc(-c5ccccc5)c4)nc(-c4ccc5ccc6ccc7ccccc7c6c5c4)n2)c13]

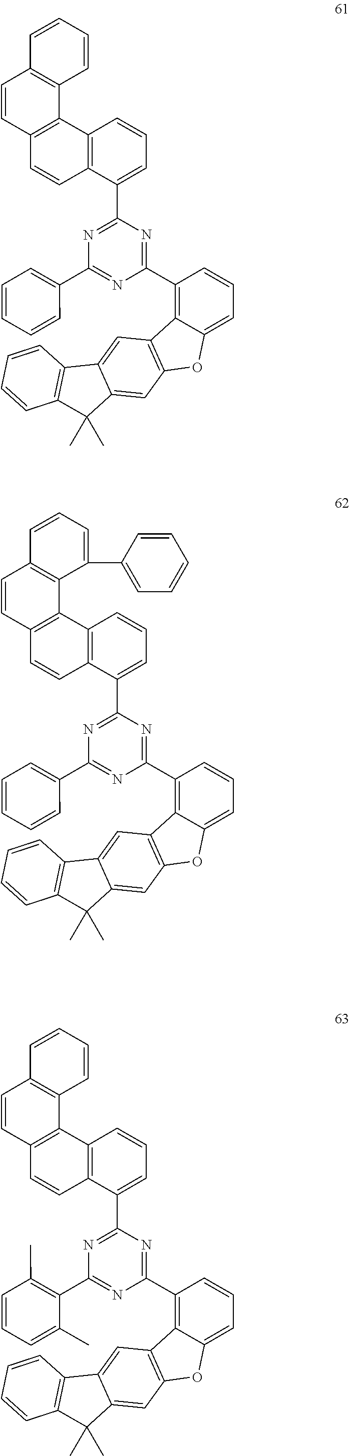 [SMILES: CC1(C)c2ccccc2-c2cc3c(cc21)oc1cccc(-c2nc(-c4ccccc4)nc(-c4cccc5c4ccc4ccc6ccccc6c45)n2)c13 ; CC1(C)c2ccccc2-c2cc3c(cc21)oc1cccc(-c2nc(-c4ccccc4)nc(-c4cccc5c4ccc4ccc6cccc(-c7ccccc7)c6c45)n2)c13 ; Cc1cccc(C)c1-c1nc(-c2cccc3c2ccc2ccc4ccccc4c23)nc(-c2cccc3oc4cc5c(cc4c23)-c2ccccc2C5(C)C)n1]

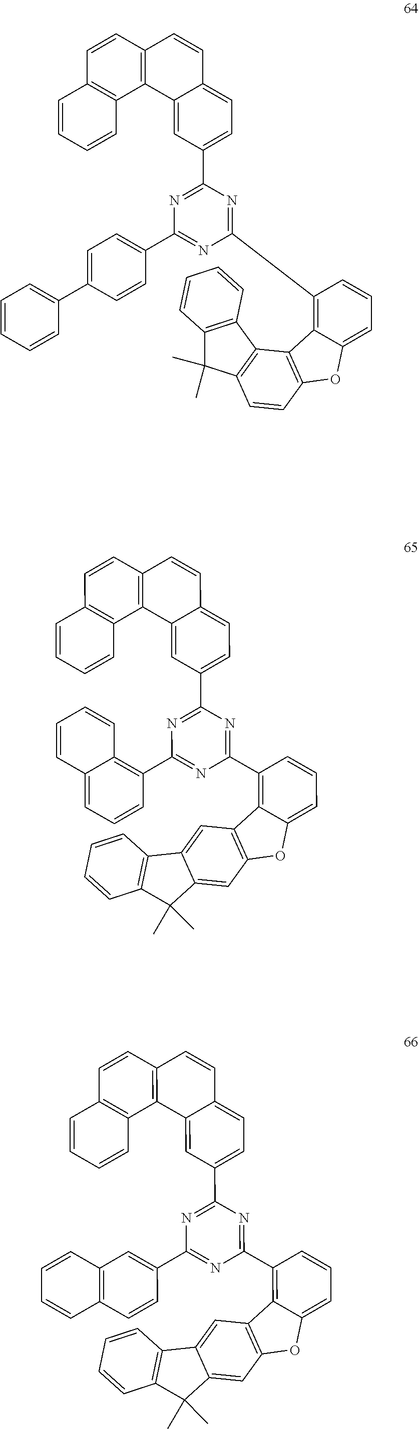 [SMILES: CC1(C)c2ccccc2-c2c1ccc1oc3cccc(-c4nc(-c5ccc(-c6ccccc6)cc5)nc(-c5ccc6ccc7ccc8ccccc8c7c6c5)n4)c3c21 ; CC1(C)c2ccccc2-c2cc3c(cc21)oc1cccc(-c2nc(-c4ccc5ccc6ccc7ccccc7c6c5c4)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6ccc7ccccc7c6c5c4)n2)c13]

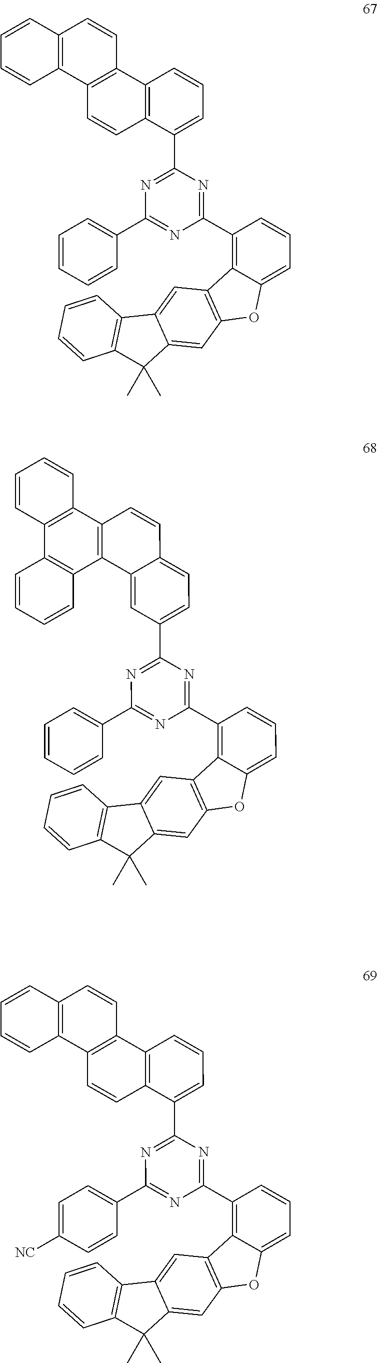 [SMILES: CC1(C)c2ccccc2-c2cc3c(cc21)oc1cccc(-c2nc(-c4ccccc4)nc(-c4cccc5c4ccc4c6ccccc6ccc54)n2)c13 ; CC1(C)c2ccccc2-c2cc3c(cc21)oc1cccc(-c2nc(-c4ccccc4)nc(-c4ccc5ccc6c7ccccc7c7ccccc7c6c5c4)n2)c13 ; CC1(C)c2ccccc2-c2cc3c(cc21)oc1cccc(-c2nc(-c4ccc(C#N)cc4)nc(-c4cccc5c4ccc4c6ccccc6ccc54)n2)c13]

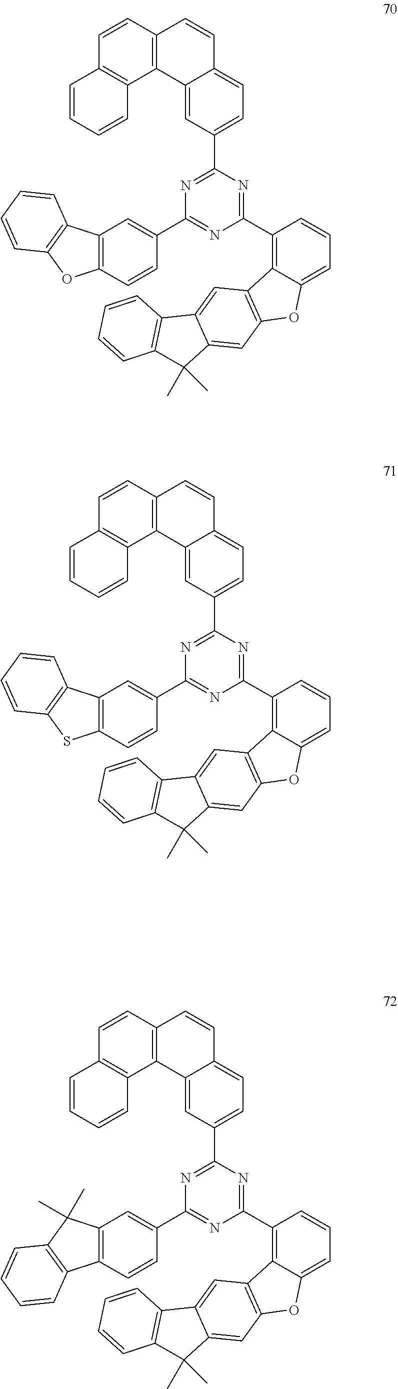 [SMILES: CC1(C)c2ccccc2-c2cc3c(cc21)oc1cccc(-c2nc(-c4ccc5oc6ccccc6c5c4)nc(-c4ccc5ccc6ccc7ccccc7c6c5c4)n2)c13 ; CC1(C)c2ccccc2-c2cc3c(cc21)oc1cccc(-c2nc(-c4ccc5sc6ccccc6c5c4)nc(-c4ccc5ccc6ccc7ccccc7c6c5c4)n2)c13 ; CC1(C)c2ccccc2-c2ccc(-c3nc(-c4ccc5ccc6ccc7ccccc7c6c5c4)nc(-c4cccc5oc6cc7c(cc6c45)-c4ccccc4C7(C)C)n3)cc21]

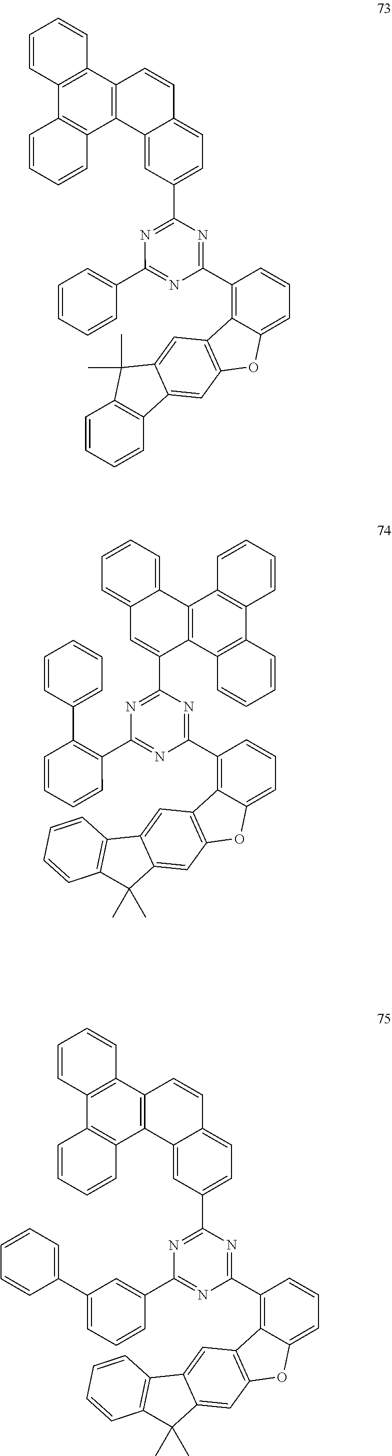 [SMILES: CC1(C)c2ccccc2-c2cc3oc4cccc(-c5nc(-c6ccccc6)nc(-c6ccc7ccc8c9ccccc9c9ccccc9c8c7c6)n5)c4c3cc21 ; CC1(C)c2ccccc2-c2cc3c(cc21)oc1cccc(-c2nc(-c4ccccc4-c4ccccc4)nc(-c4cc5ccccc5c5c6ccccc6c6ccccc6c45)n2)c13 ; CC1(C)c2ccccc2-c2cc3c(cc21)oc1cccc(-c2nc(-c4cccc(-c5ccccc5)c4)nc(-c4ccc5ccc6c7ccccc7c7ccccc7c6c5c4)n2)c13]

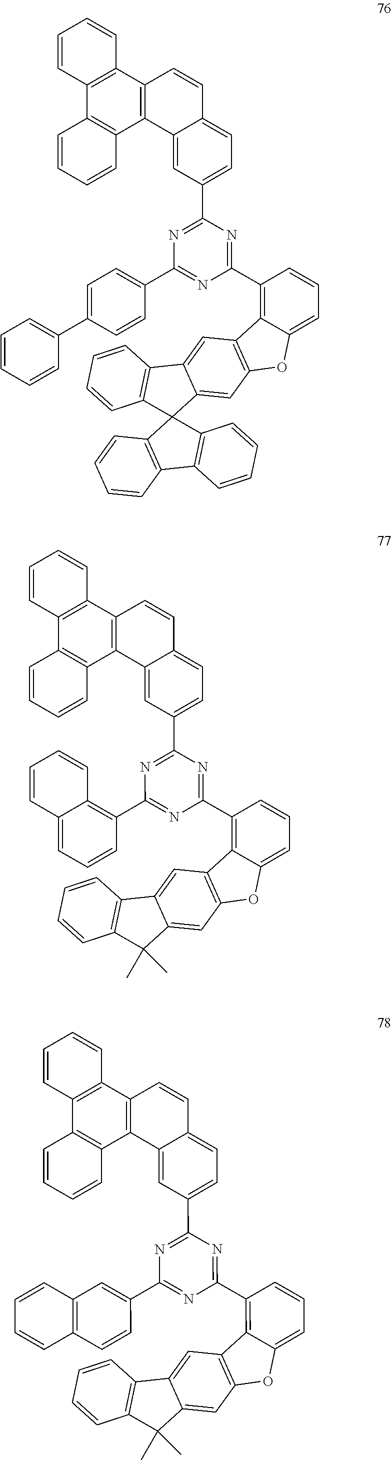 [SMILES: c1ccc(-c2ccc(-c3nc(-c4ccc5ccc6c7ccccc7c7ccccc7c6c5c4)nc(-c4cccc5oc6cc7c(cc6c45)-c4ccccc4C74c5ccccc5-c5ccccc54)n3)cc2)cc1 ; CC1(C)c2ccccc2-c2cc3c(cc21)oc1cccc(-c2nc(-c4ccc5ccc6c7ccccc7c7ccccc7c6c5c4)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6c7ccccc7c7ccccc7c6c5c4)n2)c13]

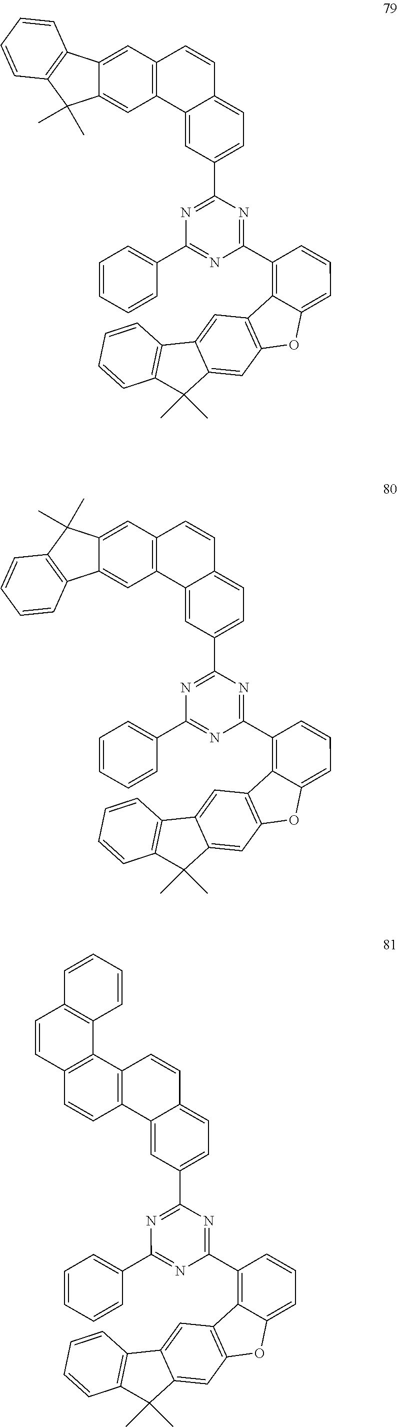 [SMILES: CC1(C)c2ccccc2-c2cc3c(cc21)oc1cccc(-c2nc(-c4ccccc4)nc(-c4ccc5ccc6cc7c(cc6c5c4)C(C)(C)c4ccccc4-7)n2)c13 ; CC1(C)c2ccccc2-c2cc3c(ccc4ccc(-c5nc(-c6ccccc6)nc(-c6cccc7oc8cc9c(cc8c67)-c6ccccc6C9(C)C)n5)cc43)cc21 ; CC1(C)c2ccccc2-c2cc3c(cc21)oc1cccc(-c2nc(-c4ccccc4)nc(-c4ccc5ccc6c(ccc7ccc8ccccc8c76)c5c4)n2)c13]

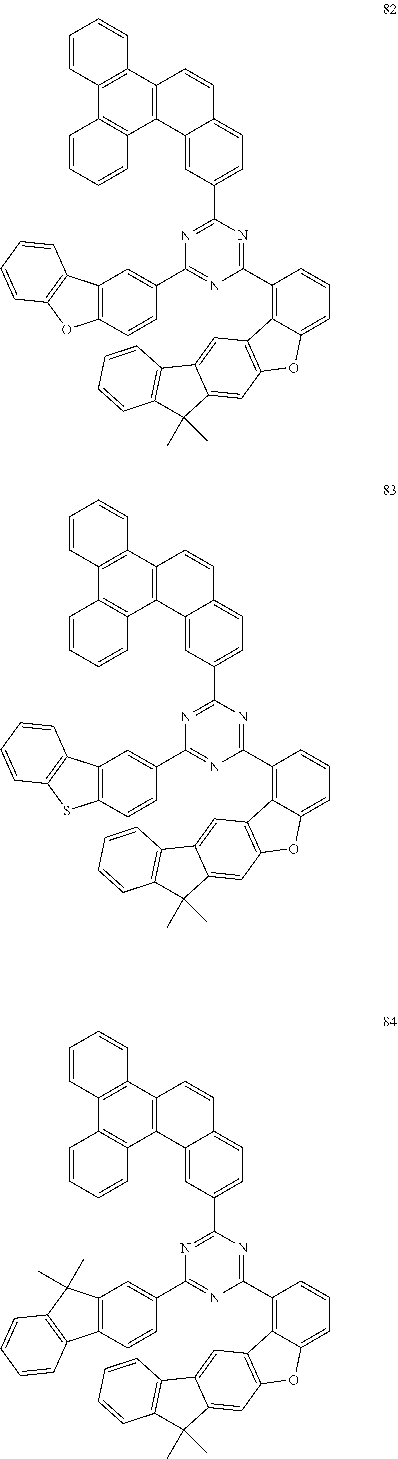 [SMILES: CC1(C)c2ccccc2-c2cc3c(cc21)oc1cccc(-c2nc(-c4ccc5oc6ccccc6c5c4)nc(-c4ccc5ccc6c7ccccc7c7ccccc7c6c5c4)n2)c13 ; CC1(C)c2ccccc2-c2cc3c(cc21)oc1cccc(-c2nc(-c4ccc5sc6ccccc6c5c4)nc(-c4ccc5ccc6c7ccccc7c7ccccc7c6c5c4)n2)c13 ; CC1(C)c2ccccc2-c2ccc(-c3nc(-c4ccc5ccc6c7ccccc7c7ccccc7c6c5c4)nc(-c4cccc5oc6cc7c(cc6c45)-c4ccccc4C7(C)C)n3)cc21]

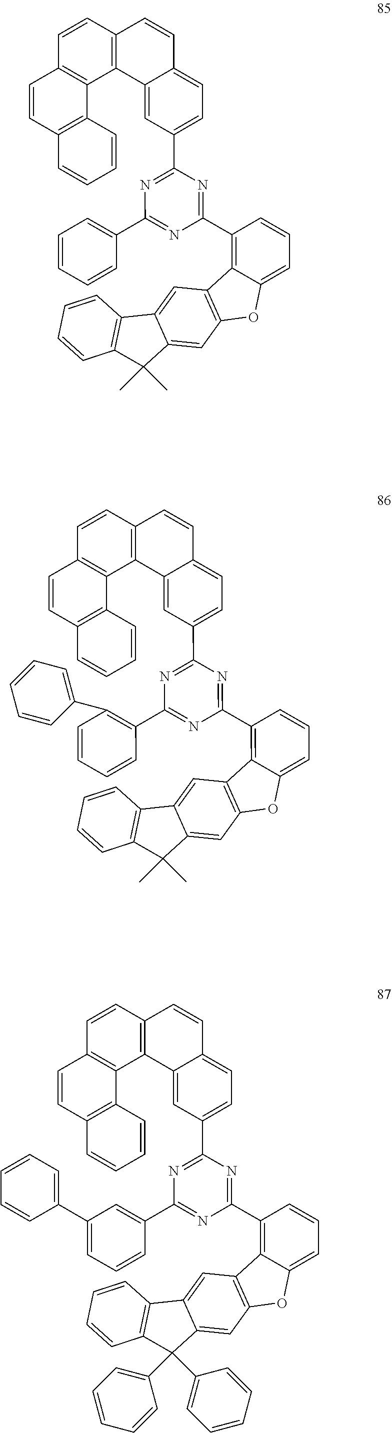 [SMILES: CC1(C)c2ccccc2-c2cc3c(cc21)oc1cccc(-c2nc(-c4ccccc4)nc(-c4ccc5ccc6ccc7ccc8ccccc8c7c6c5c4)n2)c13 ; CC1(C)c2ccccc2-c2cc3c(cc21)oc1cccc(-c2nc(-c4ccc5ccc6ccc7ccc8ccccc8c7c6c5c4)nc(-c4ccccc4-c4ccccc4)n2)c13 ; c1ccc(-c2cccc(-c3nc(-c4ccc5ccc6ccc7ccc8ccccc8c7c6c5c4)nc(-c4cccc5oc6cc7c(cc6c45)-c4ccccc4C7(c4ccccc4)c4ccccc4)n3)c2)cc1]

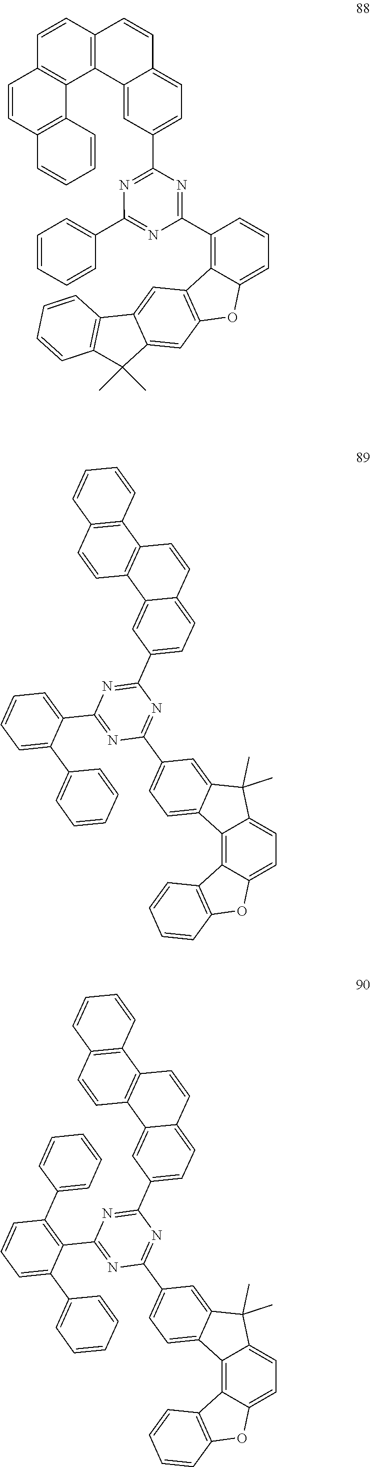 [SMILES: CC1(C)c2ccccc2-c2cc3c(cc21)oc1cccc(-c2nc(-c4ccccc4)nc(-c4ccc5ccc6ccc7ccc8ccccc8c7c6c5c4)n2)c13 ; CC1(C)c2cc(-c3nc(-c4ccc5ccc6c7ccccc7ccc6c5c4)nc(-c4ccccc4-c4ccccc4)n3)ccc2-c2c1ccc1oc3ccccc3c21 ; CC1(C)c2cc(-c3nc(-c4ccc5ccc6c7ccccc7ccc6c5c4)nc(-c4c(-c5ccccc5)cccc4-c4ccccc4)n3)ccc2-c2c1ccc1oc3ccccc3c21]

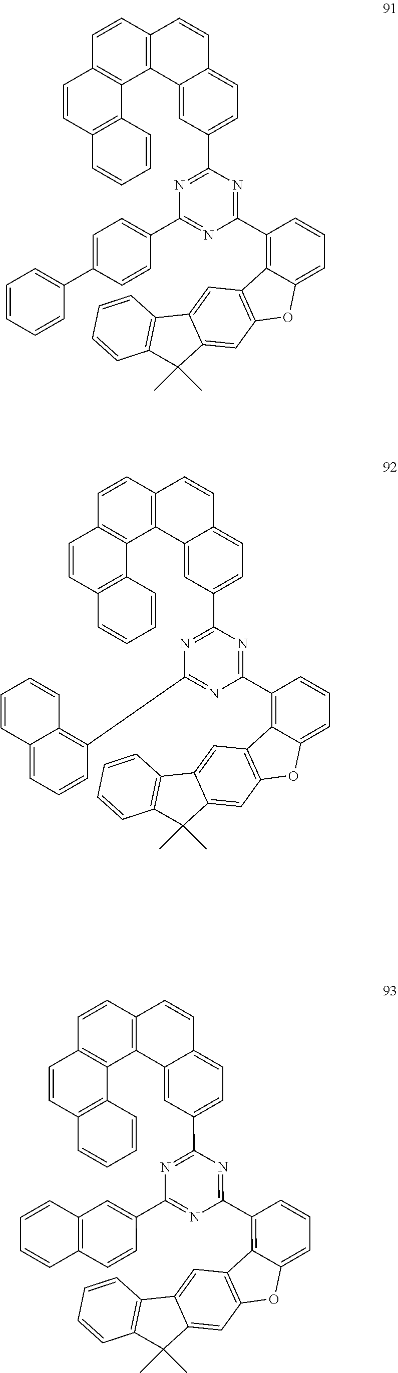 [SMILES: CC1(C)c2ccccc2-c2cc3c(cc21)oc1cccc(-c2nc(-c4ccc(-c5ccccc5)cc4)nc(-c4ccc5ccc6ccc7ccc8ccccc8c7c6c5c4)n2)c13 ; CC1(C)c2ccccc2-c2cc3c(cc21)oc1cccc(-c2nc(-c4ccc5ccc6ccc7ccc8ccccc8c7c6c5c4)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6ccc7ccc8ccccc8c7c6c5c4)n2)c13]

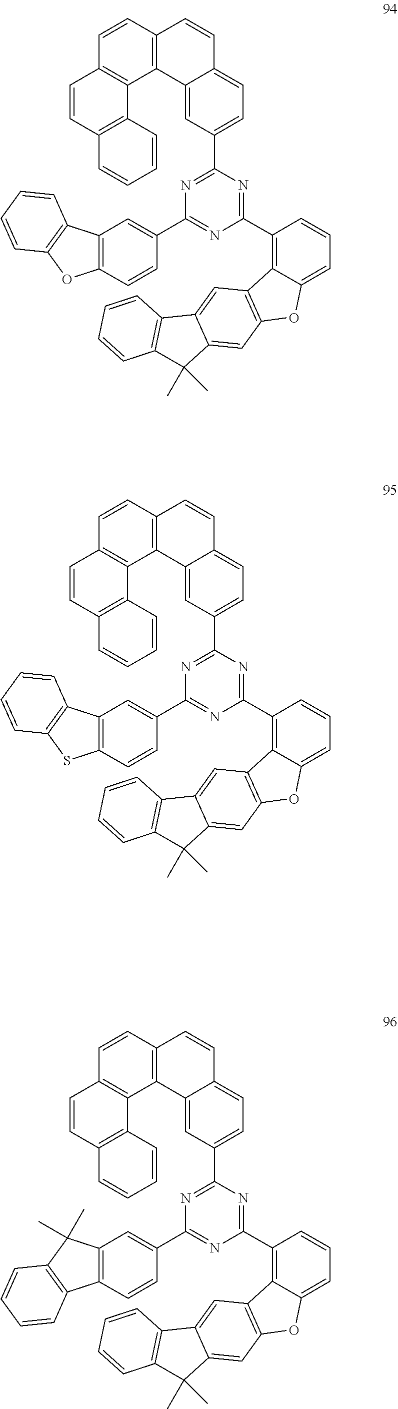 [SMILES: CC1(C)c2ccccc2-c2cc3c(cc21)oc1cccc(-c2nc(-c4ccc5oc6ccccc6c5c4)nc(-c4ccc5ccc6ccc7ccc8ccccc8c7c6c5c4)n2)c13 ; CC1(C)c2ccccc2-c2cc3c(cc21)oc1cccc(-c2nc(-c4ccc5sc6ccccc6c5c4)nc(-c4ccc5ccc6ccc7ccc8ccccc8c7c6c5c4)n2)c13 ; CC1(C)c2ccccc2-c2ccc(-c3nc(-c4ccc5ccc6ccc7ccc8ccccc8c7c6c5c4)nc(-c4cccc5oc6cc7c(cc6c45)-c4ccccc4C7(C)C)n3)cc21]

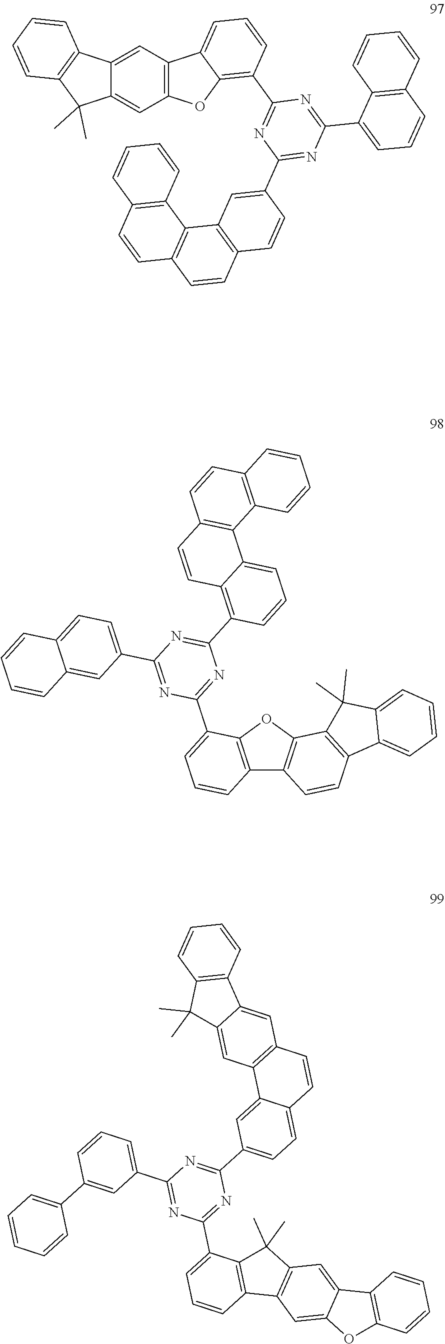 [SMILES: CC1(C)c2ccccc2-c2cc3c(cc21)oc1c(-c2nc(-c4ccc5ccc6ccc7ccccc7c6c5c4)nc(-c4cccc5ccccc45)n2)cccc13 ; CC1(C)c2ccccc2-c2ccc3c(oc4c(-c5nc(-c6ccc7ccccc7c6)nc(-c6cccc7c6ccc6ccc8ccccc8c67)n5)cccc43)c21 ; CC1(C)c2ccccc2-c2cc3ccc4ccc(-c5nc(-c6cccc(-c7ccccc7)c6)nc(-c6cccc7c6C(C)(C)c6cc8c(cc6-7)oc6ccccc68)n5)cc4c3cc21]

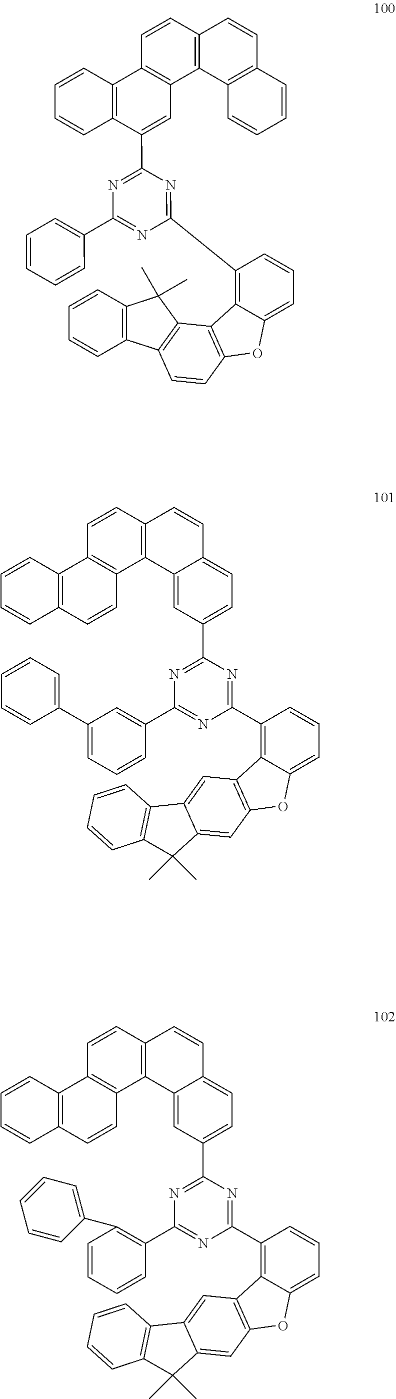 [SMILES: CC1(C)c2ccccc2-c2ccc3oc4cccc(-c5nc(-c6ccccc6)nc(-c6cc7c(ccc8ccc9ccccc9c87)c7ccccc67)n5)c4c3c21 ; CC1(C)c2ccccc2-c2cc3c(cc21)oc1cccc(-c2nc(-c4cccc(-c5ccccc5)c4)nc(-c4ccc5ccc6ccc7c8ccccc8ccc7c6c5c4)n2)c13 ; CC1(C)c2ccccc2-c2cc3c(cc21)oc1cccc(-c2nc(-c4ccc5ccc6ccc7c8ccccc8ccc7c6c5c4)nc(-c4ccccc4-c4ccccc4)n2)c13]

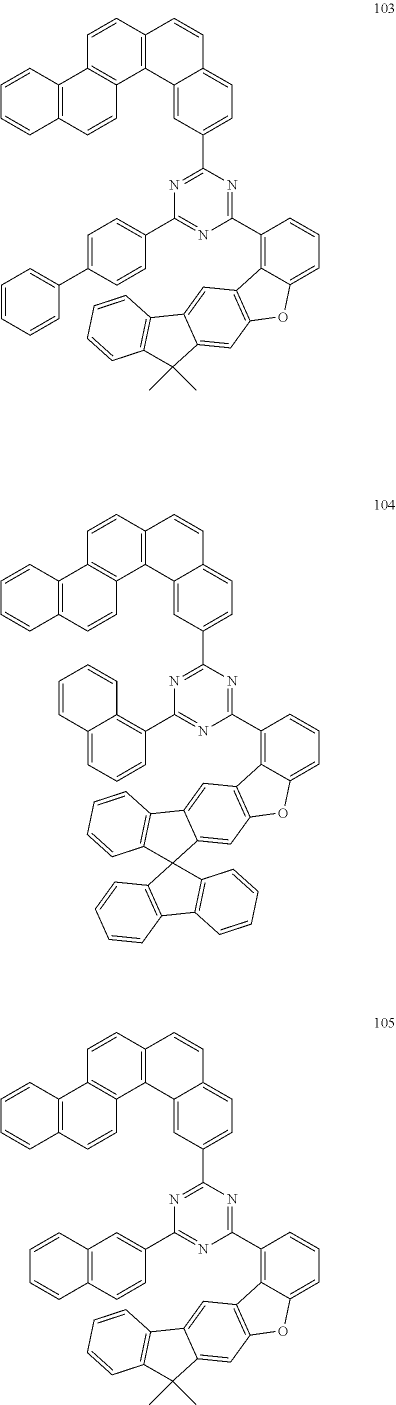

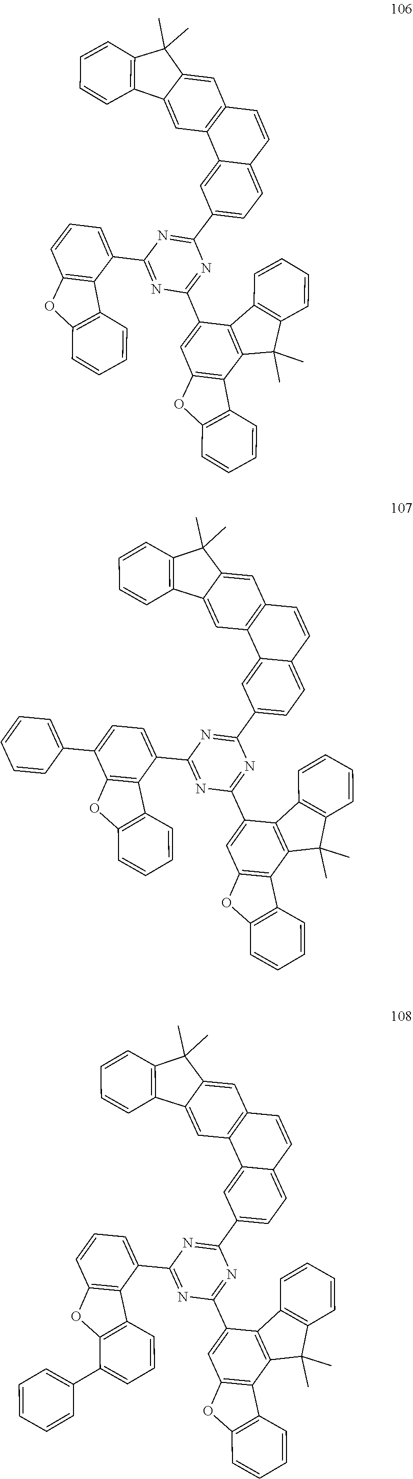 [SMILES: CC1(C)c2ccccc2-c2cc3c(ccc4ccc(-c5nc(-c6cc7oc8ccccc8c7c7c6-c6ccccc6C7(C)C)nc(-c6cccc7oc8ccccc8c67)n5)cc43)cc21 ; CC1(C)c2ccccc2-c2cc3c(ccc4ccc(-c5nc(-c6cc7oc8ccccc8c7c7c6-c6ccccc6C7(C)C)nc(-c6ccc(-c7ccccc7)c7oc8ccccc8c67)n5)cc43)cc21 ; CC1(C)c2ccccc2-c2cc3c(ccc4ccc(-c5nc(-c6cc7oc8ccccc8c7c7c6-c6ccccc6C7(C)C)nc(-c6cccc7oc8c(-c9ccccc9)cccc8c67)n5)cc43)cc21]

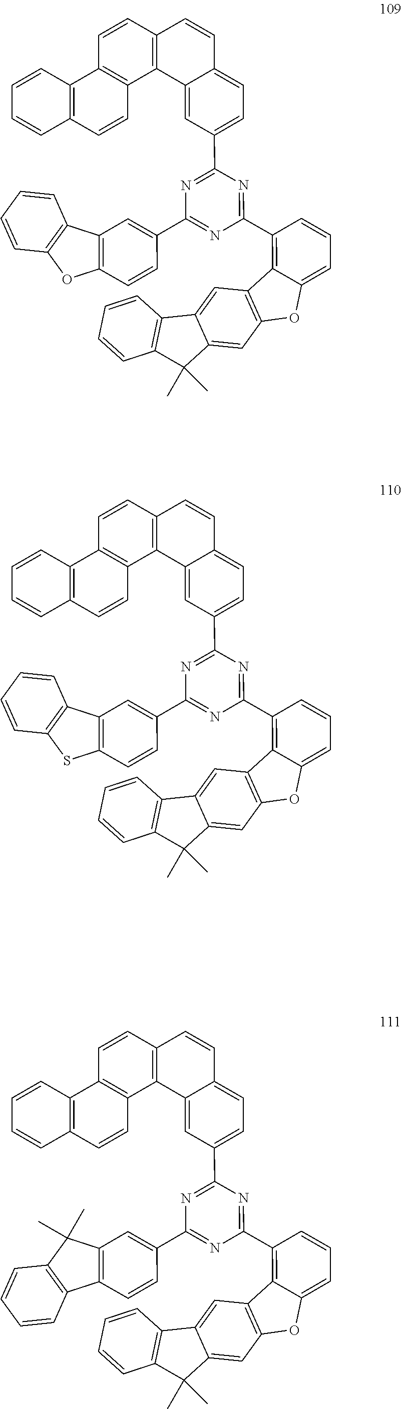 [SMILES: CC1(C)c2ccccc2-c2cc3c(cc21)oc1cccc(-c2nc(-c4ccc5oc6ccccc6c5c4)nc(-c4ccc5ccc6ccc7c8ccccc8ccc7c6c5c4)n2)c13 ; CC1(C)c2ccccc2-c2cc3c(cc21)oc1cccc(-c2nc(-c4ccc5sc6ccccc6c5c4)nc(-c4ccc5ccc6ccc7c8ccccc8ccc7c6c5c4)n2)c13 ; CC1(C)c2ccccc2-c2ccc(-c3nc(-c4ccc5ccc6ccc7c8ccccc8ccc7c6c5c4)nc(-c4cccc5oc6cc7c(cc6c45)-c4ccccc4C7(C)C)n3)cc21]

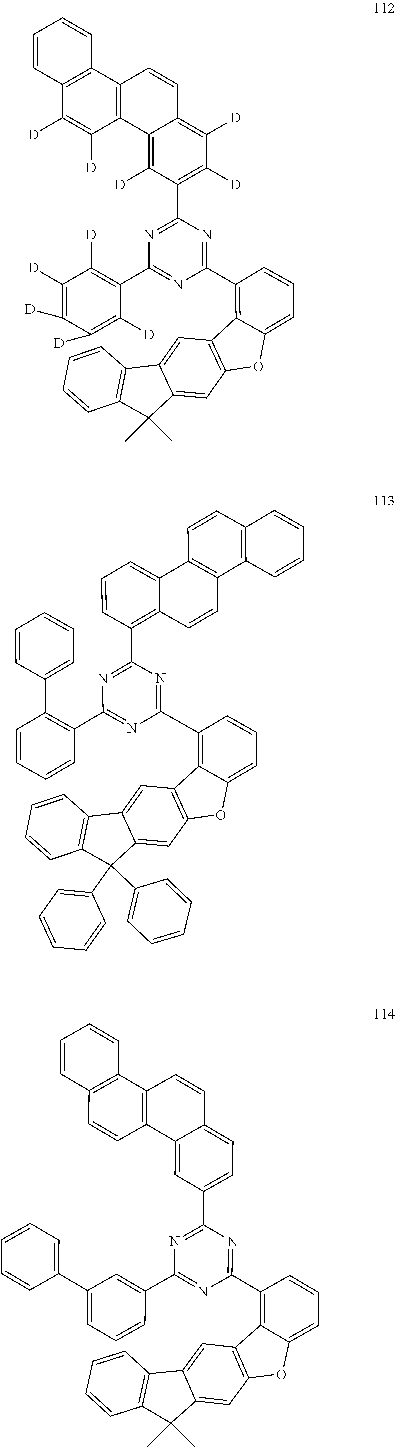 [SMILES: [2H]c1c([2H])c([2H])c(-c2nc(-c3c([2H])c([2H])c4ccc5c6ccccc6c([2H])c([2H])c5c4c3[2H])nc(-c3cccc4oc5cc6c(cc5c34)-c3ccccc3C6(C)C)n2)c([2H])c1[2H] ; c1ccc(-c2ccccc2-c2nc(-c3cccc4c3ccc3c5ccccc5ccc43)nc(-c3cccc4oc5cc6c(cc5c34)-c3ccccc3C6(c3ccccc3)c3ccccc3)n2)cc1 ; CC1(C)c2ccccc2-c2cc3c(cc21)oc1cccc(-c2nc(-c4cccc(-c5ccccc5)c4)nc(-c4ccc5ccc6c7ccccc7ccc6c5c4)n2)c13]

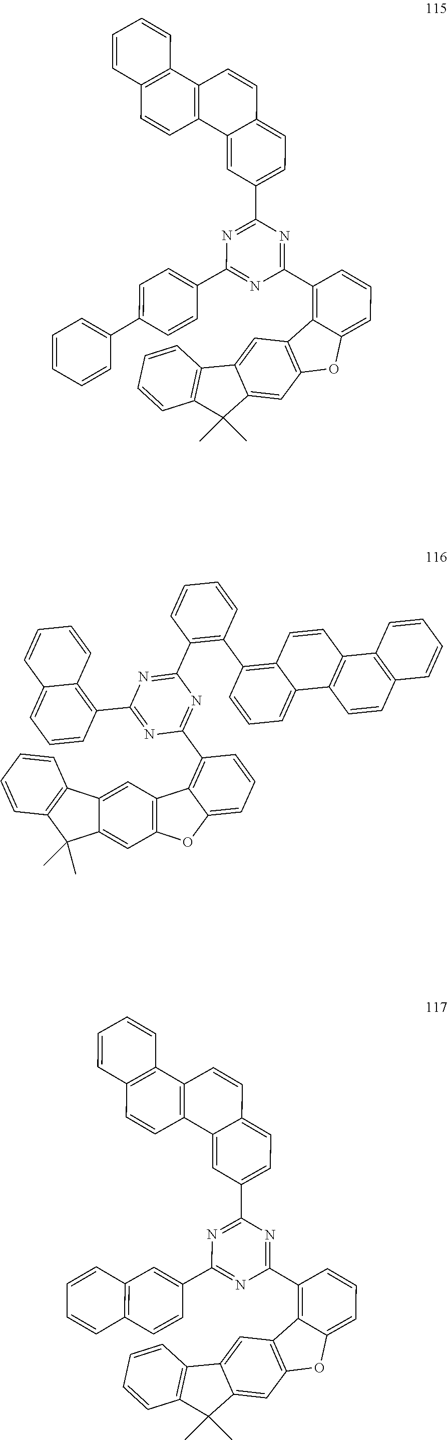 [SMILES: CC1(C)c2ccccc2-c2cc3c(cc21)oc1cccc(-c2nc(-c4ccc(-c5ccccc5)cc4)nc(-c4ccc5ccc6c7ccccc7ccc6c5c4)n2)c13 ; CC1(C)c2ccccc2-c2cc3c(cc21)oc1cccc(-c2nc(-c4ccccc4-c4cccc5c4ccc4c6ccccc6ccc54)nc(-c4cccc5ccccc45)n2)c13 ; CC1(C)c2ccccc2-c2cc3c(cc21)oc1cccc(-c2nc(-c4ccc5ccccc5c4)nc(-c4ccc5ccc6c7ccccc7ccc6c5c4)n2)c13]

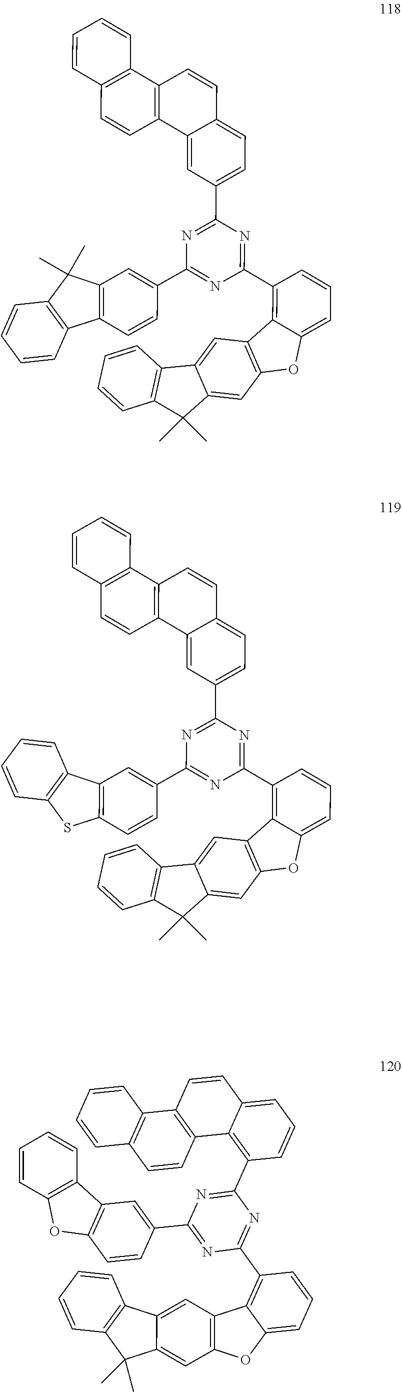 [SMILES: CC1(C)c2ccccc2-c2ccc(-c3nc(-c4ccc5ccc6c7ccccc7ccc6c5c4)nc(-c4cccc5oc6cc7c(cc6c45)-c4ccccc4C7(C)C)n3)cc21 ; CC1(C)c2ccccc2-c2cc3c(cc21)oc1cccc(-c2nc(-c4ccc5ccc6c7ccccc7ccc6c5c4)nc(-c4ccc5sc6ccccc6c5c4)n2)c13 ; CC1(C)c2ccccc2-c2cc3c(cc21)oc1cccc(-c2nc(-c4ccc5oc6ccccc6c5c4)nc(-c4cccc5ccc6c7ccccc7ccc6c45)n2)c13]

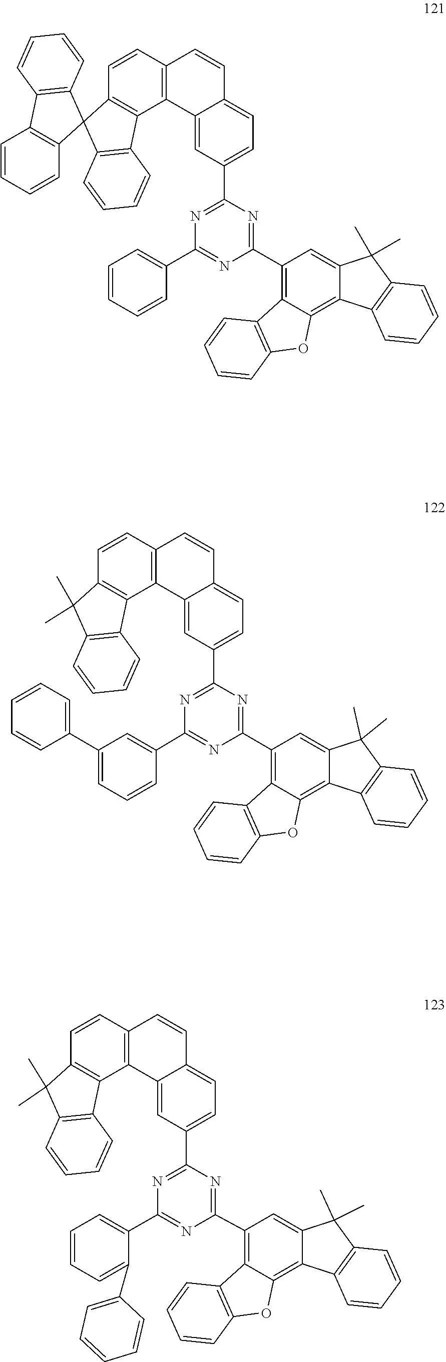 [SMILES: CC1(C)c2ccccc2-c2c1cc(-c1nc(-c3ccccc3)nc(-c3ccc4ccc5ccc6c(c5c4c3)-c3ccccc3C63c4ccccc4-c4ccccc43)n1)c1c2oc2ccccc21 ; CC1(C)c2ccccc2-c2c1cc(-c1nc(-c3cccc(-c4ccccc4)c3)nc(-c3ccc4ccc5ccc6c(c5c4c3)-c3ccccc3C6(C)C)n1)c1c2oc2ccccc21 ; CC1(C)c2ccccc2-c2c1cc(-c1nc(-c3ccc4ccc5ccc6c(c5c4c3)-c3ccccc3C6(C)C)nc(-c3ccccc3-c3ccccc3)n1)c1c2oc2ccccc21]

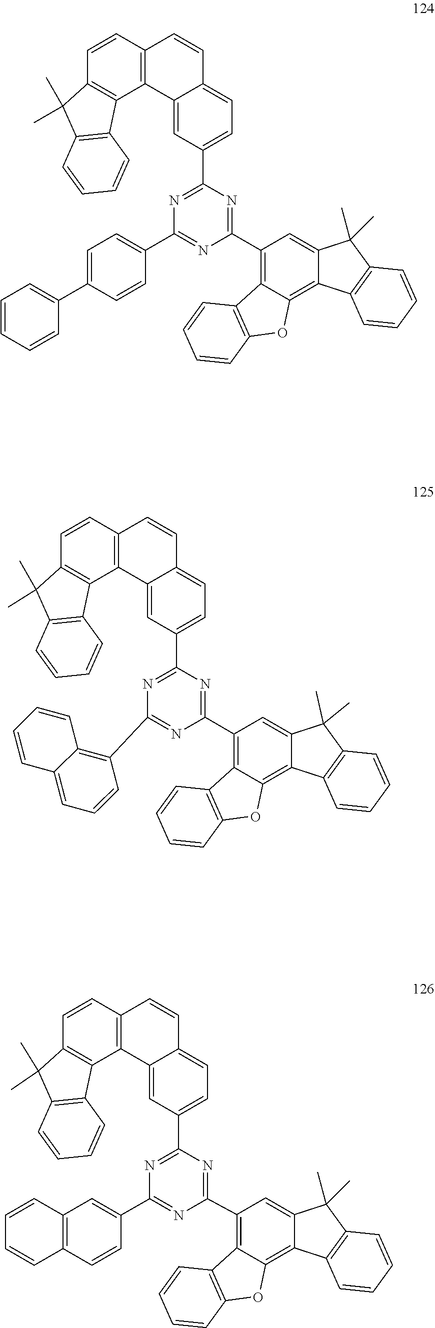 [SMILES: CC1(C)c2ccccc2-c2c1cc(-c1nc(-c3ccc(-c4ccccc4)cc3)nc(-c3ccc4ccc5ccc6c(c5c4c3)-c3ccccc3C6(C)C)n1)c1c2oc2ccccc21 ; CC1(C)c2ccccc2-c2c1cc(-c1nc(-c3ccc4ccc5ccc6c(c5c4c3)-c3ccccc3C6(C)C)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6c(c5c4c3)-c3ccccc3C6(C)C)n1)c1c2oc2ccccc21]

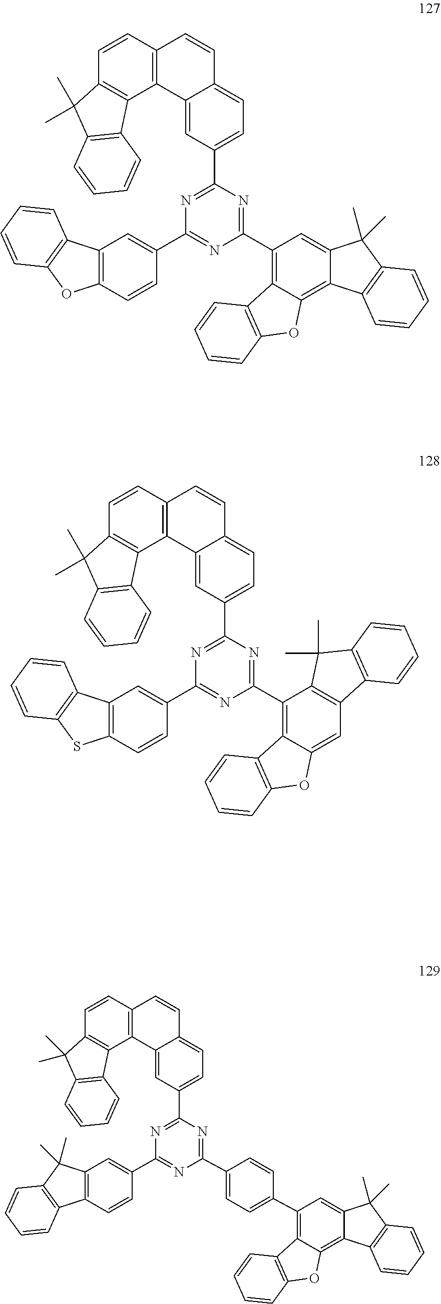 [SMILES: CC1(C)c2ccccc2-c2c1cc(-c1nc(-c3ccc4oc5ccccc5c4c3)nc(-c3ccc4ccc5ccc6c(c5c4c3)-c3ccccc3C6(C)C)n1)c1c2oc2ccccc21 ; CC1(C)c2ccccc2-c2c1ccc1ccc3ccc(-c4nc(-c5ccc6sc7ccccc7c6c5)nc(-c5c6c(cc7oc8ccccc8c57)-c5ccccc5C6(C)C)n4)cc3c21 ; CC1(C)c2ccccc2-c2ccc(-c3nc(-c4ccc(-c5cc6c(c7oc8ccccc8c57)-c5ccccc5C6(C)C)cc4)nc(-c4ccc5ccc6ccc7c(c6c5c4)-c4ccccc4C7(C)C)n3)cc21]

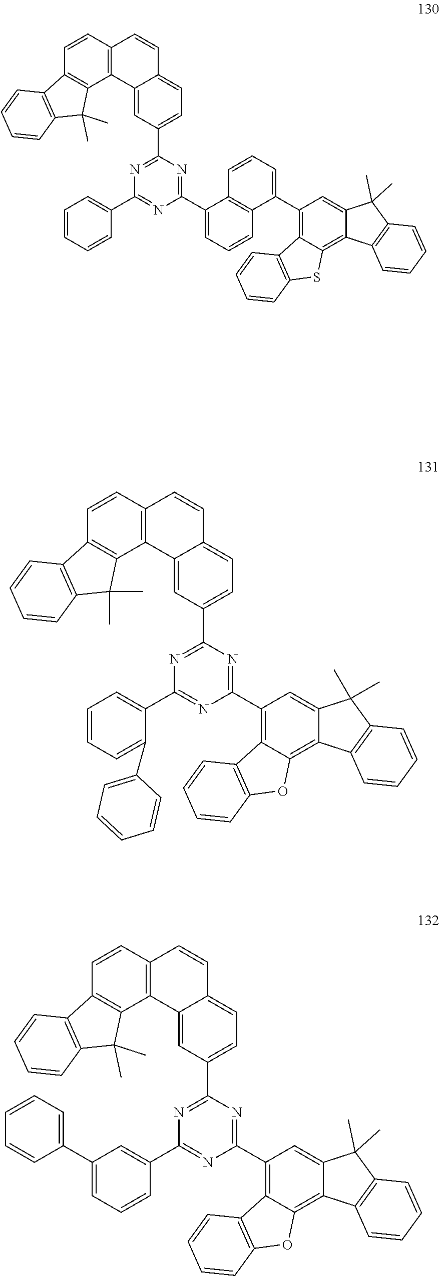 [SMILES: CC1(C)c2ccccc2-c2c1cc(-c1cccc3c(-c4nc(-c5ccccc5)nc(-c5ccc6ccc7ccc8c(c7c6c5)C(C)(C)c5ccccc5-8)n4)cccc13)c1c2sc2ccccc21 ; CC1(C)c2ccccc2-c2c1cc(-c1nc(-c3ccc4ccc5ccc6c(c5c4c3)C(C)(C)c3ccccc3-6)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5ccc6c(c5c4c3)C(C)(C)c3ccccc3-6)n1)c1c2oc2ccccc21]

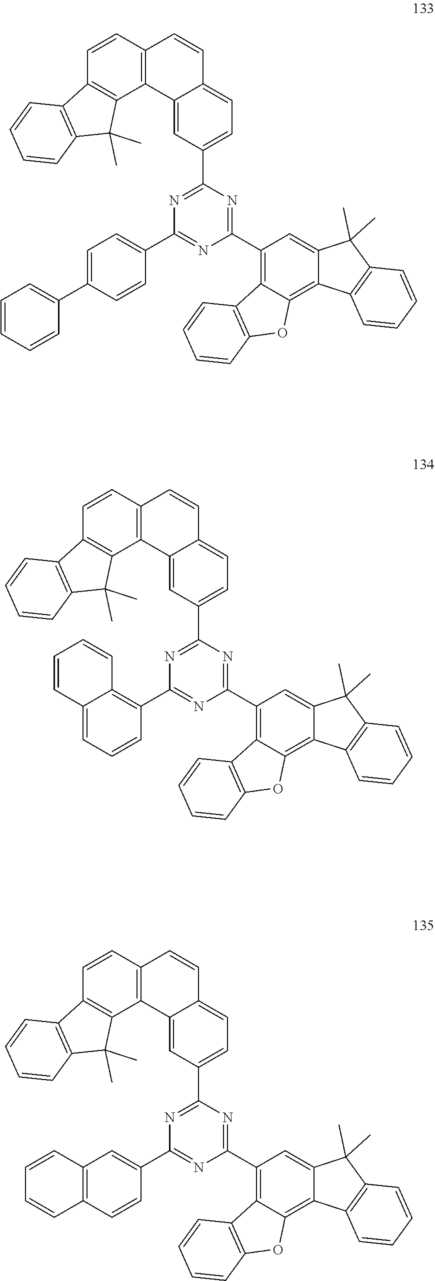 [SMILES: CC1(C)c2ccccc2-c2c1cc(-c1nc(-c3ccc(-c4ccccc4)cc3)nc(-c3ccc4ccc5ccc6c(c5c4c3)C(C)(C)c3ccccc3-6)n1)c1c2oc2ccccc21 ; CC1(C)c2ccccc2-c2c1cc(-c1nc(-c3ccc4ccc5ccc6c(c5c4c3)C(C)(C)c3ccccc3-6)nc(-c3cccc4ccccc34)n1)c1c2oc2ccccc21 ; CC1(C)c2ccccc2-c2c1cc(-c1nc(-c3ccc4ccccc4c3)nc(-c3ccc4ccc5ccc6c(c5c4c3)C(C)(C)c3ccccc3-6)n1)c1c2oc2ccccc21]

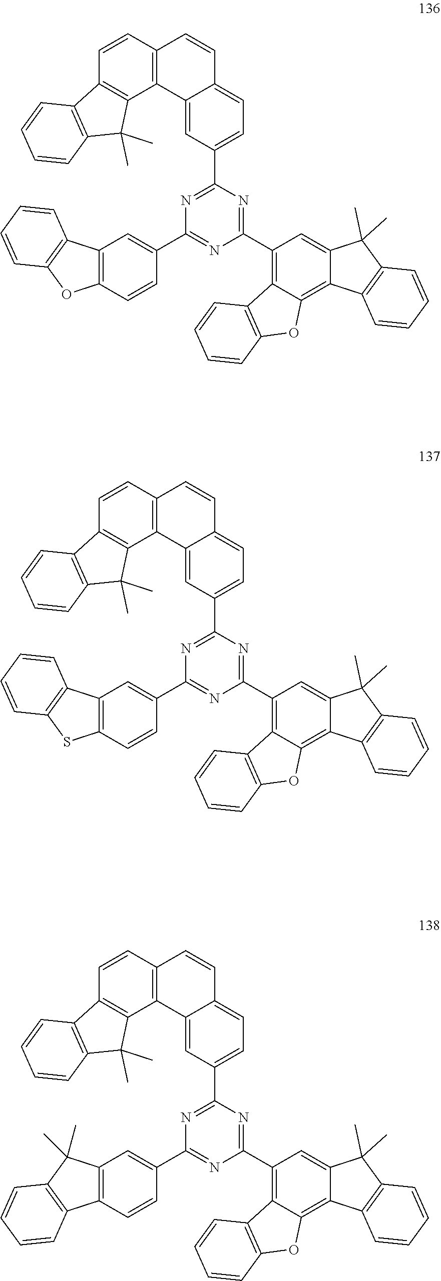 [SMILES: CC1(C)c2ccccc2-c2c1cc(-c1nc(-c3ccc4oc5ccccc5c4c3)nc(-c3ccc4ccc5ccc6c(c5c4c3)C(C)(C)c3ccccc3-6)n1)c1c2oc2ccccc21 ; CC1(C)c2ccccc2-c2c1cc(-c1nc(-c3ccc4sc5ccccc5c4c3)nc(-c3ccc4ccc5ccc6c(c5c4c3)C(C)(C)c3ccccc3-6)n1)c1c2oc2ccccc21 ; CC1(C)c2ccccc2-c2ccc(-c3nc(-c4ccc5ccc6ccc7c(c6c5c4)C(C)(C)c4ccccc4-7)nc(-c4cc5c(c6oc7ccccc7c46)-c4ccccc4C5(C)C)n3)cc21]

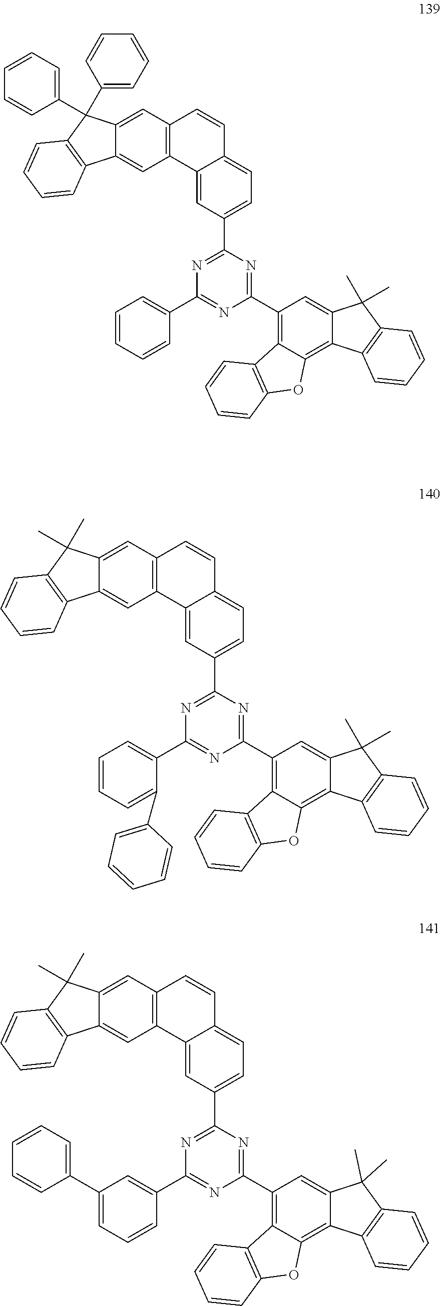 [SMILES: CC1(C)c2ccccc2-c2c1cc(-c1nc(-c3ccccc3)nc(-c3ccc4ccc5cc6c(cc5c4c3)-c3ccccc3C6(c3ccccc3)c3ccccc3)n1)c1c2oc2ccccc21 ; CC1(C)c2ccccc2-c2cc3c(ccc4ccc(-c5nc(-c6ccccc6-c6ccccc6)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6cccc(-c7ccccc7)c6)nc(-c6cc7c(c8oc9ccccc9c68)-c6ccccc6C7(C)C)n5)cc43)cc21]

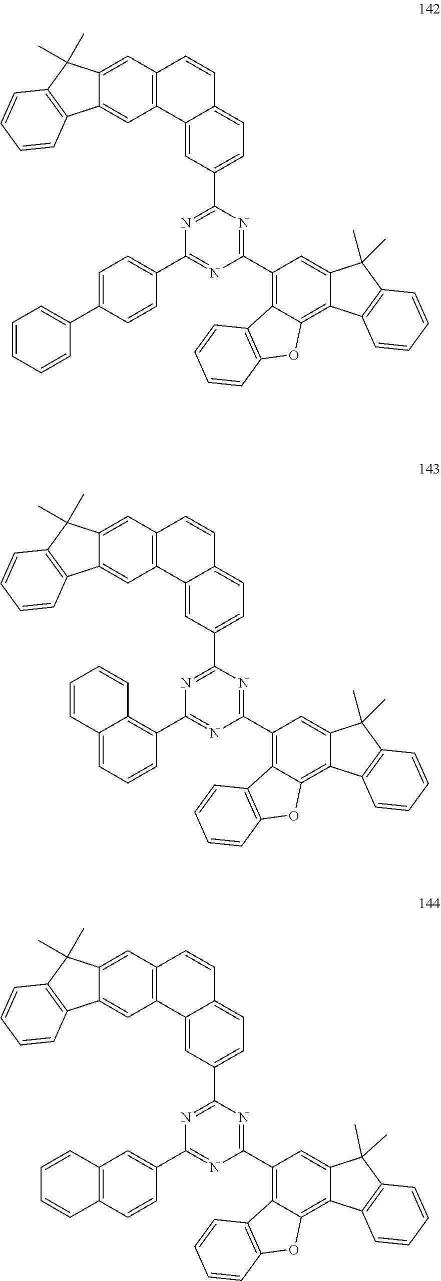 [SMILES: CC1(C)c2ccccc2-c2cc3c(ccc4ccc(-c5nc(-c6ccc(-c7ccccc7)cc6)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6cccc7ccccc67)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6ccc7ccccc7c6)nc(-c6cc7c(c8oc9ccccc9c68)-c6ccccc6C7(C)C)n5)cc43)cc21]

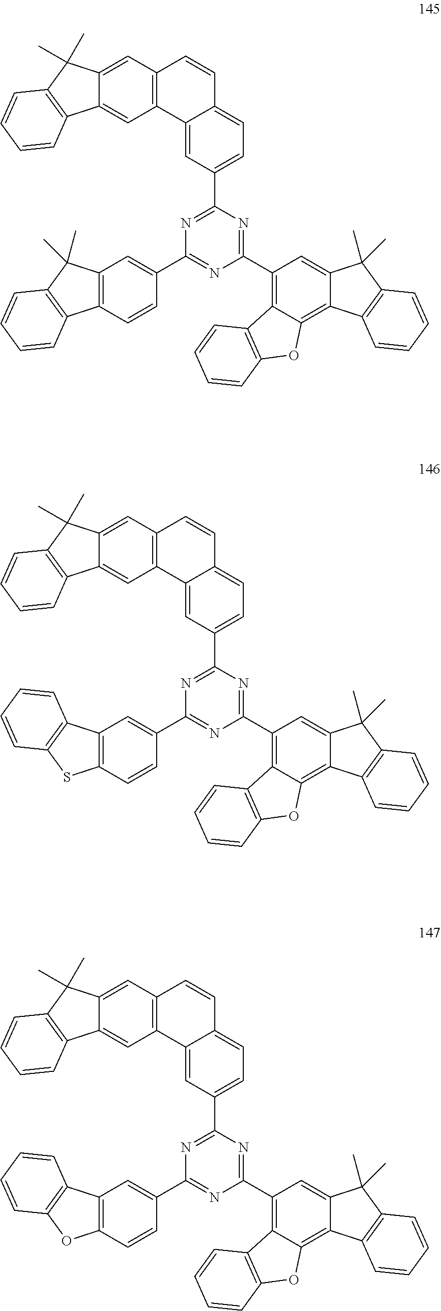 [SMILES: CC1(C)c2ccccc2-c2ccc(-c3nc(-c4ccc5ccc6cc7c(cc6c5c4)-c4ccccc4C7(C)C)nc(-c4cc5c(c6oc7ccccc7c46)-c4ccccc4C5(C)C)n3)cc21 ; CC1(C)c2ccccc2-c2cc3c(ccc4ccc(-c5nc(-c6ccc7sc8ccccc8c7c6)nc(-c6cc7c(c8oc9ccccc9c68)-c6ccccc6C7(C)C)n5)cc43)cc21 ; CC1(C)c2ccccc2-c2cc3c(ccc4ccc(-c5nc(-c6ccc7oc8ccccc8c7c6)nc(-c6cc7c(c8oc9ccccc9c68)-c6ccccc6C7(C)C)n5)cc43)cc21]

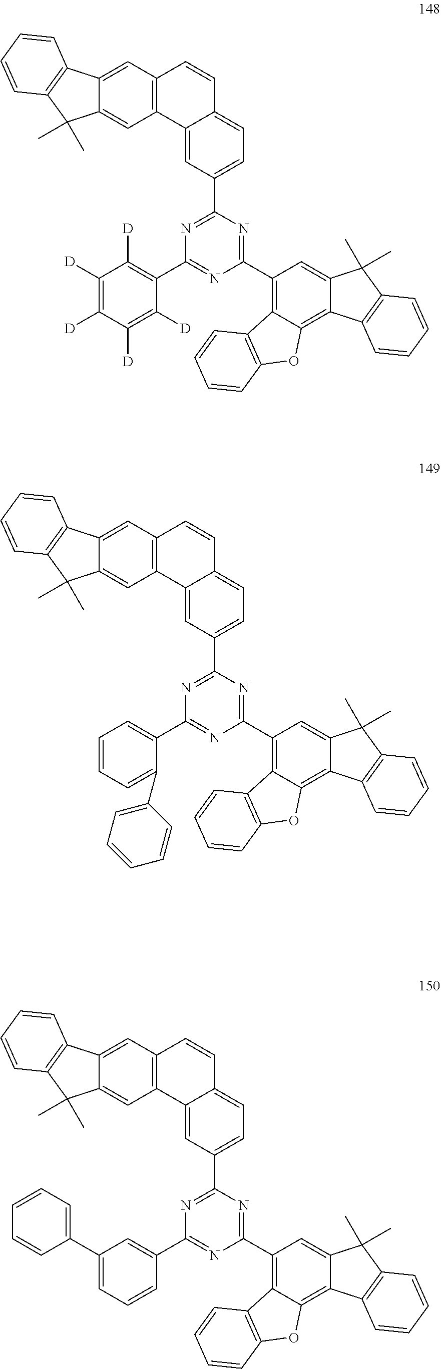 [SMILES: [2H]c1c([2H])c([2H])c(-c2nc(-c3ccc4ccc5cc6c(cc5c4c3)C(C)(C)c3ccccc3-6)nc(-c3cc4c(c5oc6ccccc6c35)-c3ccccc3C4(C)C)n2)c([2H])c1[2H] ; CC1(C)c2ccccc2-c2cc3ccc4ccc(-c5nc(-c6ccccc6-c6ccccc6)nc(-c6cc7c(c8oc9ccccc9c68)-c6ccccc6C7(C)C)n5)cc4c3cc21 ; CC1(C)c2ccccc2-c2cc3ccc4ccc(-c5nc(-c6cccc(-c7ccccc7)c6)nc(-c6cc7c(c8oc9ccccc9c68)-c6ccccc6C7(C)C)n5)cc4c3cc21]

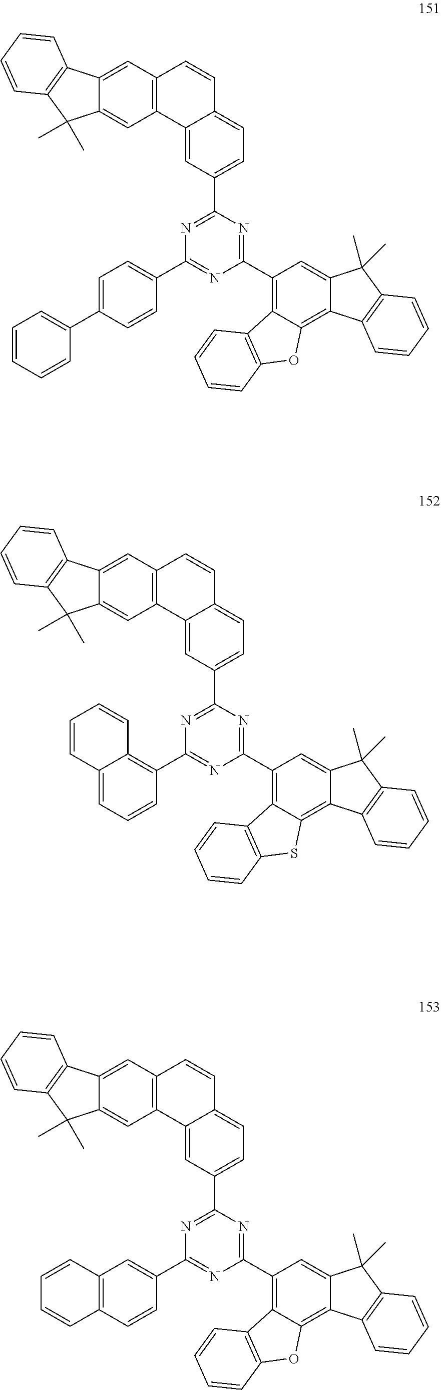 [SMILES: CC1(C)c2ccccc2-c2cc3ccc4ccc(-c5nc(-c6ccc(-c7ccccc7)cc6)nc(-c6cc7c(c8oc9ccccc9c68)-c6ccccc6C7(C)C)n5)cc4c3cc21 ; CC1(C)c2ccccc2-c2cc3ccc4ccc(-c5nc(-c6cccc7ccccc67)nc(-c6cc7c(c8sc9ccccc9c68)-c6ccccc6C7(C)C)n5)cc4c3cc21 ; CC1(C)c2ccccc2-c2cc3ccc4ccc(-c5nc(-c6ccc7ccccc7c6)nc(-c6cc7c(c8oc9ccccc9c68)-c6ccccc6C7(C)C)n5)cc4c3cc21]

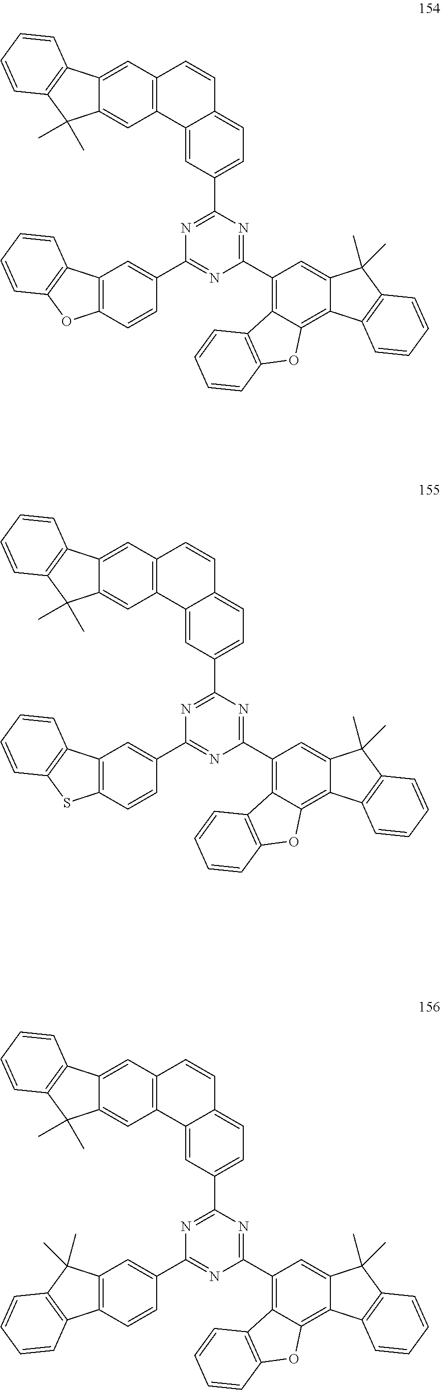 [SMILES: CC1(C)c2ccccc2-c2cc3ccc4ccc(-c5nc(-c6ccc7oc8ccccc8c7c6)nc(-c6cc7c(c8oc9ccccc9c68)-c6ccccc6C7(C)C)n5)cc4c3cc21 ; CC1(C)c2ccccc2-c2cc3ccc4ccc(-c5nc(-c6ccc7sc8ccccc8c7c6)nc(-c6cc7c(c8oc9ccccc9c68)-c6ccccc6C7(C)C)n5)cc4c3cc21 ; CC1(C)c2ccccc2-c2ccc(-c3nc(-c4ccc5ccc6cc7c(cc6c5c4)C(C)(C)c4ccccc4-7)nc(-c4cc5c(c6oc7ccccc7c46)-c4ccccc4C5(C)C)n3)cc21]

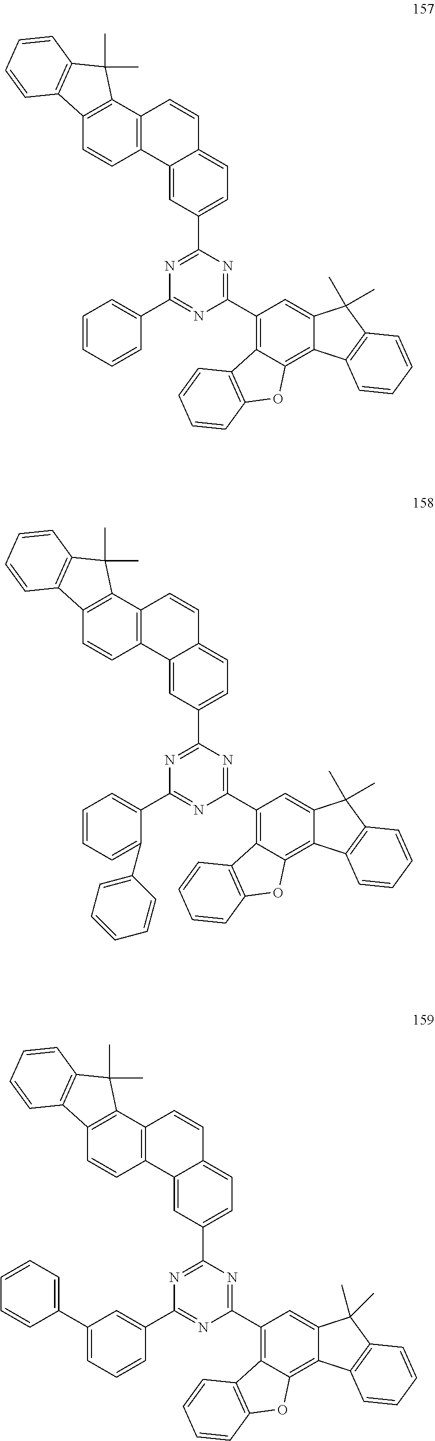 [SMILES: CC1(C)c2ccccc2-c2c1cc(-c1nc(-c3ccccc3)nc(-c3ccc4ccc5c6c(ccc5c4c3)-c3ccccc3C6(C)C)n1)c1c2oc2ccccc21 ; CC1(C)c2ccccc2-c2c1cc(-c1nc(-c3ccc4ccc5c6c(ccc5c4c3)-c3ccccc3C6(C)C)nc(-c3ccccc3-c3ccccc3)n1)c1c2oc2ccccc21 ; CC1(C)c2ccccc2-c2c1cc(-c1nc(-c3cccc(-c4ccccc4)c3)nc(-c3ccc4ccc5c6c(ccc5c4c3)-c3ccccc3C6(C)C)n1)c1c2oc2ccccc21]

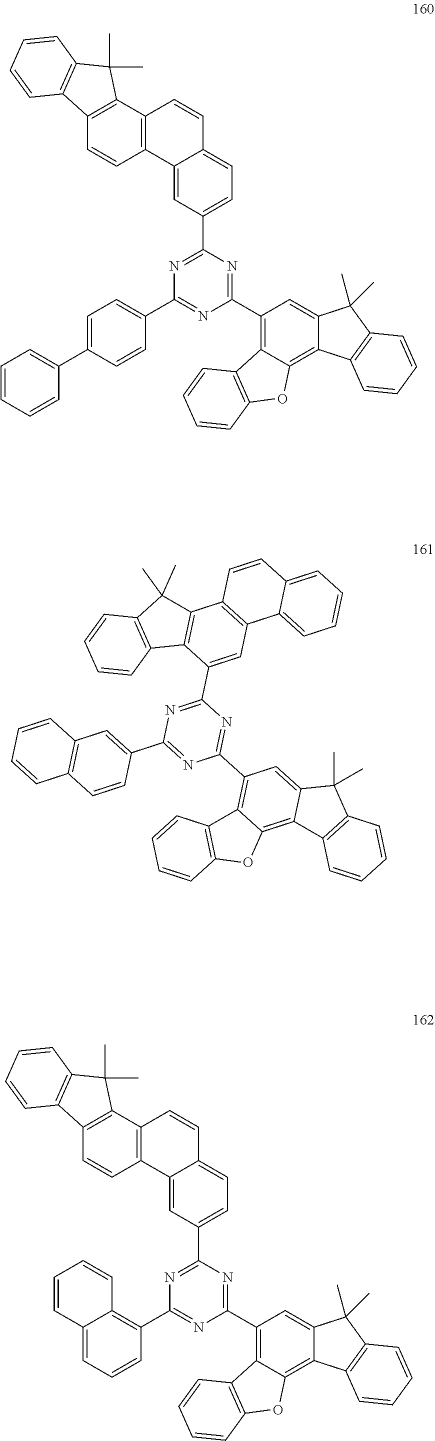 [SMILES: CC1(C)c2ccccc2-c2c1cc(-c1nc(-c3ccc(-c4ccccc4)cc3)nc(-c3ccc4ccc5c6c(ccc5c4c3)-c3ccccc3C6(C)C)n1)c1c2oc2ccccc21 ; CC1(C)c2ccccc2-c2c1cc(-c1nc(-c3ccc4ccccc4c3)nc(-c3cc4c(ccc5ccccc54)c4c3-c3ccccc3C4(C)C)n1)c1c2oc2ccccc21 ; CC1(C)c2ccccc2-c2c1cc(-c1nc(-c3ccc4ccc5c6c(ccc5c4c3)-c3ccccc3C6(C)C)nc(-c3cccc4ccccc34)n1)c1c2oc2ccccc21]

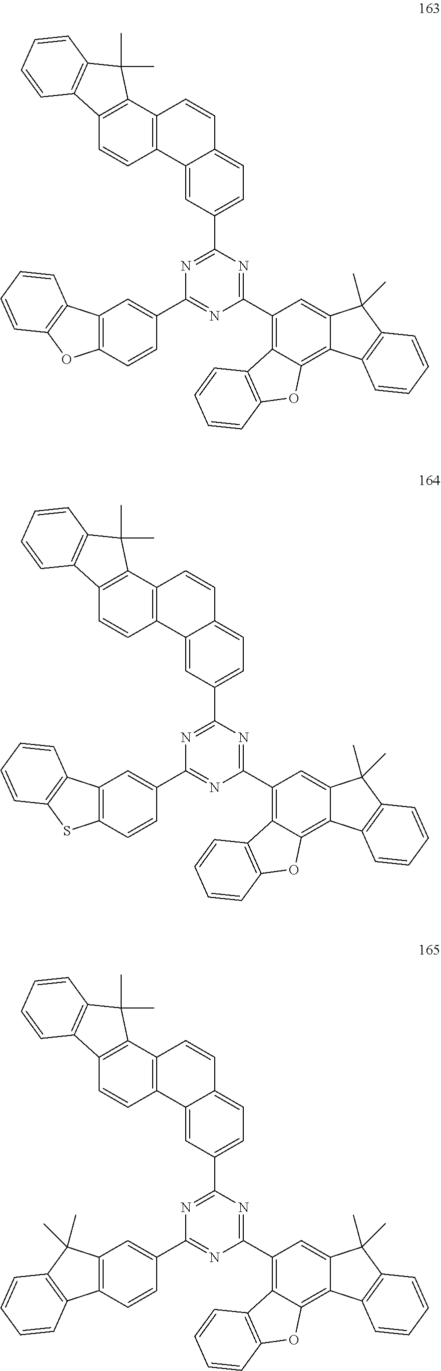 [SMILES: CC1(C)c2ccccc2-c2c1cc(-c1nc(-c3ccc4ccc5c6c(ccc5c4c3)-c3ccccc3C6(C)C)nc(-c3ccc4oc5ccccc5c4c3)n1)c1c2oc2ccccc21 ; CC1(C)c2ccccc2-c2c1cc(-c1nc(-c3ccc4ccc5c6c(ccc5c4c3)-c3ccccc3C6(C)C)nc(-c3ccc4sc5ccccc5c4c3)n1)c1c2oc2ccccc21 ; CC1(C)c2ccccc2-c2ccc(-c3nc(-c4ccc5ccc6c7c(ccc6c5c4)-c4ccccc4C7(C)C)nc(-c4cc5c(c6oc7ccccc7c46)-c4ccccc4C5(C)C)n3)cc21]

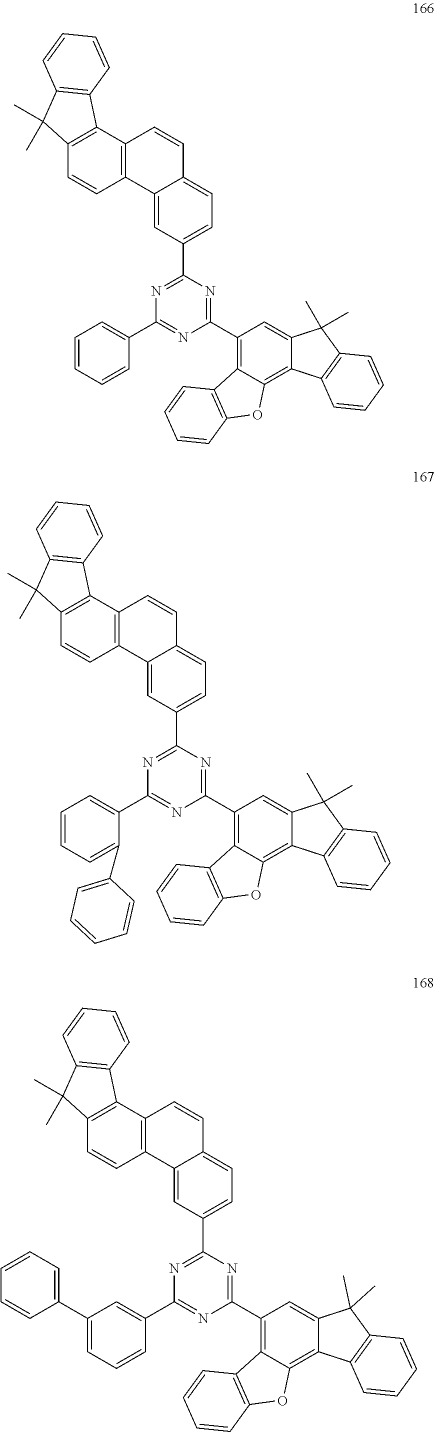 [SMILES: CC1(C)c2ccccc2-c2c1ccc1c2ccc2ccc(-c3nc(-c4ccccc4)nc(-c4cc5c(c6oc7ccccc7c46)-c4ccccc4C5(C)C)n3)cc21 ; CC1(C)c2ccccc2-c2c1ccc1c2ccc2ccc(-c3nc(-c4ccccc4-c4ccccc4)nc(-c4cc5c(c6oc7ccccc7c46)-c4ccccc4C5(C)C)n3)cc21 ; CC1(C)c2ccccc2-c2c1ccc1c2ccc2ccc(-c3nc(-c4cccc(-c5ccccc5)c4)nc(-c4cc5c(c6oc7ccccc7c46)-c4ccccc4C5(C)C)n3)cc21]

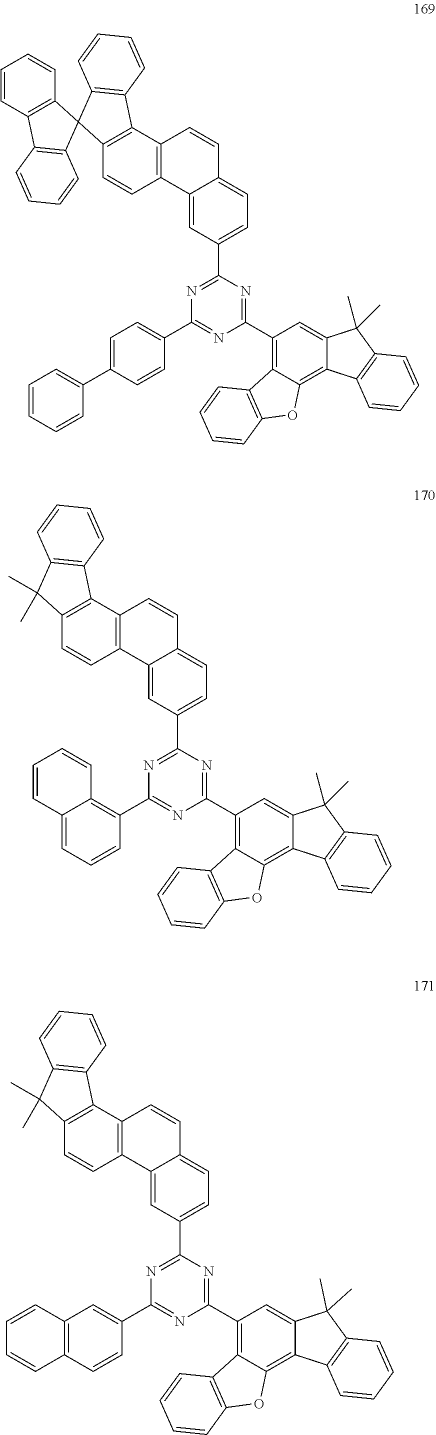

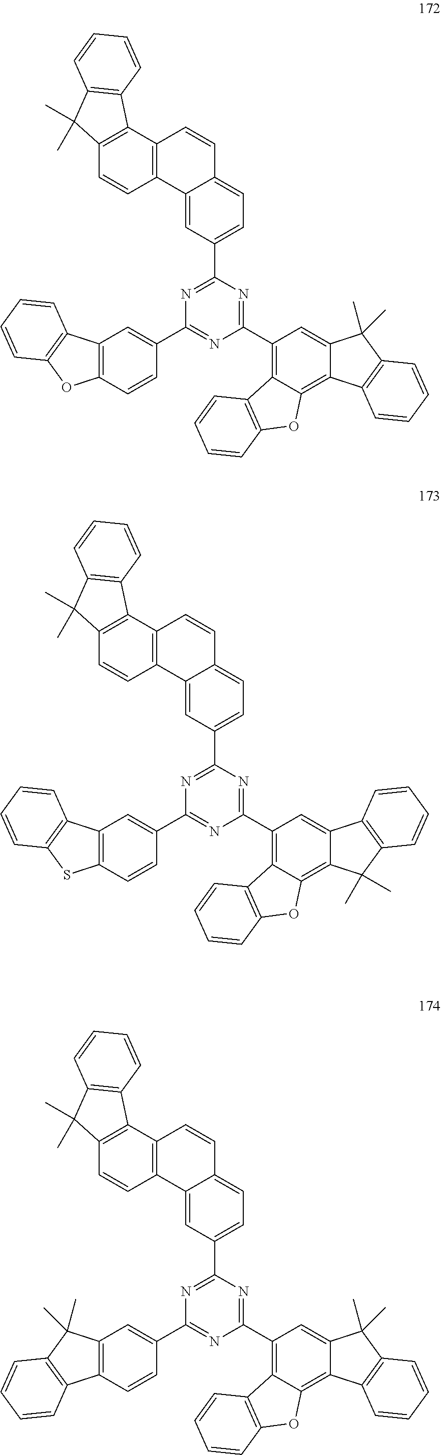 [SMILES: CC1(C)c2ccccc2-c2c1ccc1c2ccc2ccc(-c3nc(-c4ccc5oc6ccccc6c5c4)nc(-c4cc5c(c6oc7ccccc7c46)-c4ccccc4C5(C)C)n3)cc21 ; CC1(C)c2ccccc2-c2c1ccc1c2ccc2ccc(-c3nc(-c4ccc5sc6ccccc6c5c4)nc(-c4cc5c(c6oc7ccccc7c46)C(C)(C)c4ccccc4-5)n3)cc21 ; CC1(C)c2ccccc2-c2ccc(-c3nc(-c4ccc5ccc6c7c(ccc6c5c4)C(C)(C)c4ccccc4-7)nc(-c4cc5c(c6oc7ccccc7c46)-c4ccccc4C5(C)C)n3)cc21]

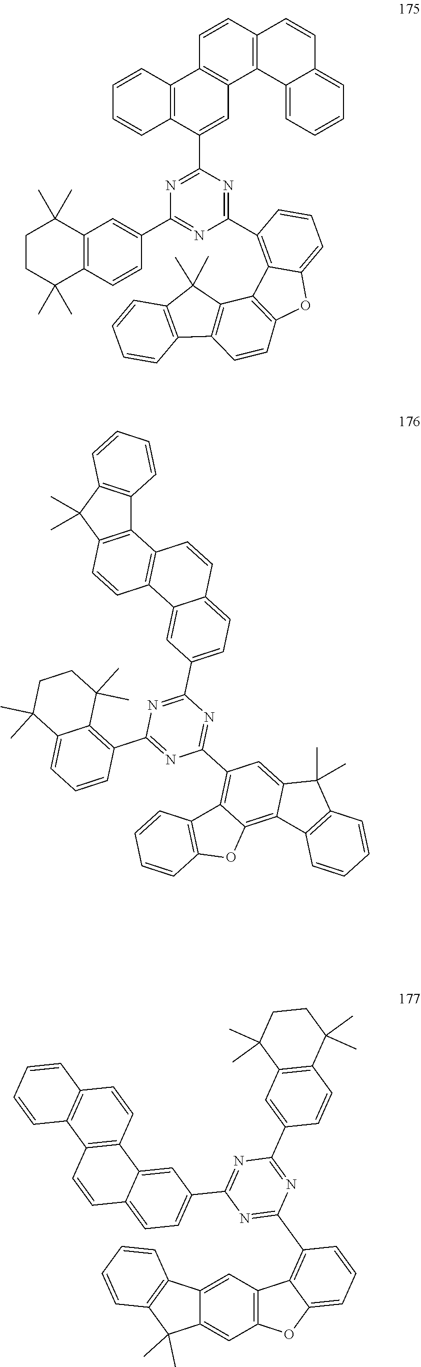 [SMILES: CC1(C)CCC(C)(C)c2cc(-c3nc(-c4cc5c(ccc6ccc7ccccc7c65)c5ccccc45)nc(-c4cccc5oc6ccc7c(c6c45)C(C)(C)c4ccccc4-7)n3)ccc21 ; CC1(C)CCC(C)(C)c2c(-c3nc(-c4ccc5ccc6c7c(ccc6c5c4)C(C)(C)c4ccccc4-7)nc(-c4cc5c(c6oc7ccccc7c46)-c4ccccc4C5(C)C)n3)cccc21 ; CC1(C)CCC(C)(C)c2cc(-c3nc(-c4ccc5ccc6c7ccccc7ccc6c5c4)nc(-c4cccc5oc6cc7c(cc6c45)-c4ccccc4C7(C)C)n3)ccc21]

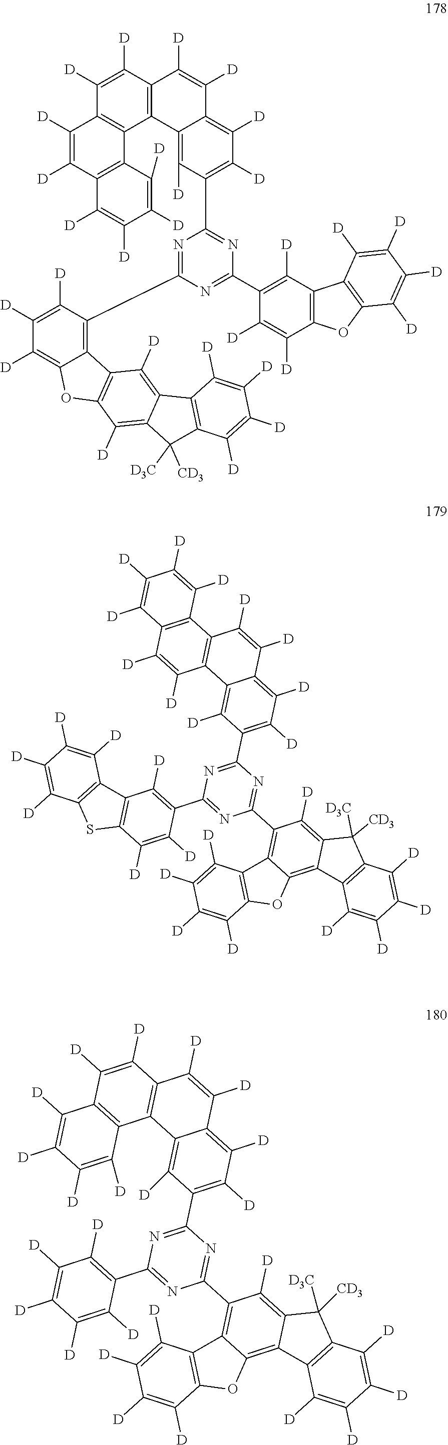

6. An organic light emitting device comprising a first electrode, a second electrode opposite to the first electrode, and one or more organic layers interposed between the first and second electrodes wherein one of the organic layers comprises the organic compound according to claim 1.

7. The organic light emitting device according to claim 6, wherein the organic layers comprise a hole injecting layer, a hole transport layer, an electron blocking layer, a functional layer having functions of both hole injection and hole transport, a light emitting layer, an electron transport layer, an electron injecting layer, a hole blocking layer, and/or a functional layer having functions of both electron injection and electron transport.

8. The organic light emitting device according to claim 7, wherein the light emitting layer is composed of a host and a dopant and the compound represented by Formula 1 is used as the host.

9. The organic light emitting device according to claim 8, wherein one or more host compounds other than the organic compound represented by Formula 1 are mixed or stacked in the light emitting layer.

10. The organic light emitting device according to claim 8, wherein the organic compound represented by Formula 1 is used as a host for red phosphorescence.

11. The organic light emitting device according to claim 8, wherein the dopant comprises at least one organometallic compound.

12. The organic light emitting device according to claim 8, wherein one or more compounds other than the organometallic compound are mixed or stacked in the light emitting layer.

13. The organic light emitting device according to claim 7, wherein each of the organic layers is formed by a deposition or solution process.

14. The organic light emitting device according to claim 6, wherein the organic light emitting device is used in a display or lighting system selected from flat panel displays, flexible displays, monochromatic flat panel lighting systems, white flat panel lighting systems, flexible monochromatic lighting systems, flexible white lighting systems, displays for automotive applications, displays for virtual reality, and displays for augmented reality.
